(19) United States
(12) Patent Application Publication
(10) Pub. No.: US 2025/0114446 A1
(43) Pub. Date: Apr. 10, 2025

(54) LOW-SUGAR CORONAVIRUS VACCINE AND METHODS THEREOF

(71) Applicant: Rock BioMedical Inc., Taipei City (TW)

(72) Inventors: Chung-Yi WU, Taipei City (TW); Jeng-Shin LEE, Lincoln, MA (US); Chi-Huey WONG, Rancho Santa Fe, CA (US)

(21) Appl. No.: 18/909,767

(22) Filed: Oct. 8, 2024

Related U.S. Application Data
(60) Provisional application No. 63/588,932, filed on Oct. 9, 2023.
(60) Provisional application No. 63/549,343, filed on Feb. 2, 2024.
(60) Provisional application No. 63/575,093, filed on Apr. 5, 2024.

(51) Int. Cl. A61K 39/215; A61K 9/127; A61K 9/51; A61K 39/00; A61P 31/14; C07K 14/005; C12N 7/00

(52) U.S. Cl. CPC A61K 39/215; A61K 9/127; A61K 9/5123; A61P 31/14; C07K 14/005; C12N 7/00; A61K 2039/53; A61K 2039/55555; A61K 2039/575; C12N 2770/20011; C12N 2770/20022; C12N 2770/20034

(57) ABSTRACT

The present disclosure relates to a low glycosylated spike protein and a vaccine designed to express the spike protein in vivo. The present disclosure also teaches a method for generating an immune response by utilizing the low glycosylated spike protein, which provides a broader protection across different variants. A method for identifying a glycan-shielded conserved peptide of a glycoprotein is also disclosed.

### CROSS-REFERENCE TO RELATED APPLICATIONS

This application claims benefit of priority to U.S. Provisional Patent Application No. 63/588,932, filed on Oct. 9, 2023, U.S. Provisional Patent Application No. 63/549,343 filed on Feb. 2, 2024, U.S. Provisional Patent Application No. 63/575,093 filed on Apr. 5, 2024, and PCT Patent Application No. PCT/US24/23597 filed on Apr. 8, 2024, the contents of which are hereby incorporated by reference in their entirety.

### SEQUENCE LISTING

The instant application contains a Sequence Listing which is submitted electronically in ASCII format and is hereby incorporated by reference in its entirety. The ASCII copy, created on Oct. 4, 2024, is named “A1000-01400US_20241004_SeqListing” and is 70,975 bytes in size.

### FIELD

The present disclosure is related to methods and compositions for treating and preventing infectious diseases, particularly diseases or symptoms caused by coronavirus infections. The present disclosure is also related to methods and compositions for treating and preventing infectious diseases using messenger RNA (mRNA) technologies.

### BACKGROUND OF THE INVENTION

Since the outbreak of severe acute respiratory syndrome due to coronavirus-2 (SARS-COV-2) in December 2019 that caused widespread Coronavirus Induced Disease 2019 (COVID-19), the virus has spread all over the world and caused more than 200 million infections and 4 million deaths. Although the pandemic has been blunted afterworld-wide inoculation of vaccines, the evolving disease continues to pose risks to certain cohorts with a weaker immune system, and future potential coronavirus outbreaks are still possible.

Great efforts have been made directed towards the development of effective vaccines to combat this pandemic, mostly by targeting the trimeric spike(S) protein on the viral surfaces. Of the various vaccines developed to control the spread of SARS-COV-2, vaccines based on mRNA technologies, such as the mRNA vaccines developed by Moderna and BioNTech/Pfizer, represent a major breakthrough due to their speed and convenience. However, while the dominant SARS-COV-2 variants have changed and evolved over time, immune escape still poses serious challenges to existing medicines and vaccines. Accordingly, there is an immediate need for improved vaccines and approaches to provide broader protection over different emerging viral variants.

### BRIEF SUMMARY OF THE INVENTION

One aspect of the present disclosure provides a nucleic acid, configured to encode a recombinant spike protein, wherein the recombinant spike protein has an N-linked glycosylation site in an S1 domain or an S2 domain thereof, provided that a stem region thereof is devoid of an N-linked glycosylation site.

One aspect of the present disclosure provides an expression vector, comprising the nucleic acid of the present disclosure.

One aspect of the present disclosure provides a composition (e.g., a vaccine composition) comprising an expression vector of the present disclosure.

One aspect of the present disclosure provides a method for generating an immune response against coronavirus infection, comprising administering an effective amount of a nucleic acid of the present disclosure to a subject in need thereof.

One aspect of the present disclosure provides a recombinant protein, comprising an amino acid sequence at least 80%, 85%, 90%, 91%, 92%, 93%, 94%, 95%, 96%, 97%, 98%, or 99%, or 100% identical to SEQ ID NO: 02, wherein X denotes any amino acid, provided that at least one of the X17, X61, X74, X122, X149, X165, X234, X282, X331, X343, X394, X487, X603, X616, and X657 is an Asn (N) residue, and at least one of X709, X717, X801, X1074, X1098, X1108, X1134, X1158, and X1173 is not an Asn (N) residue; SEQ ID NO: 02, wherein X denotes any amino acid, provided that at least one of the X17, X61, X74, X122, X149, X165, X234, X282, X331, X343, X394, X487, X603, X616, X657, X709, X717, and X801 is an Asn (N) N residue, and at least one of the X1074, X1098, X1108, X1134, X1158, and X1173 is not an Asn (N) N residue; SEQ ID NO: 05, wherein X denotes any amino acid, provided that at least one of X23, X31, X115, X388, X412, X422, X448, X472, and X487 is not an Asn (N) residue; SEQ ID NO: 07, wherein X denotes any amino acid, provided that at least one of the X12, X36, X46, X72, X96, and X111 is not an Asn (N) residue; SEQ ID NO: 13, wherein X denotes any amino acid, provided that at least one of the X61, X74, X122, X149, X163, X232, X280, X329, X341, X392, X485, X601, X614, and X655 is an Asn (N) residue, and at least one of the X707, X715, X799, X1072, X1096, X1106, X1132, X1156, and X1171 is not an Asn (N) residue; SEQ ID NO: 13, wherein X denotes any amino acid, provided that at least one of X61, X74, X122, X149, X163, X232, X280, X329, X341, X392, X485, X601, X614, X655, X707, X715, and X799 is an Asn (N) residue, and at least one of X1072, X1096, X1106, X1132, X1156, and X1171 is not an Asn (N) residue; SEQ ID NO: 16, wherein X denotes any amino acid, provided that at least one of X23, X31, X115, X388, X412, X422, X448, X472, and X487 is not an Asn (N) residue; or SEQ ID NO: 18, wherein X denotes any amino acid, provided that at least one of the X12, X36, X46, X72, X96, and X111 is not an Asn (N) residue.

One aspect of the present disclosure provides an isolated immunogenic peptide comprising at least one amino acid sequence selected from a group consisting of: SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, and SEQ ID NO: 39.

One aspect of the present disclosure provides a recombinant spike protein comprising a first plurality of peptides, each comprises an amino acid sequence selected from a group consisting of SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33; and a second plurality of peptides, each comprises an amino acid sequence selected from a group consisting of SEQ ID NO: 34, SEQ ID NO: 35, SEQ ID NO: 36, SEQ ID NO: 37, SEQ ID NO: 38, and SEQ ID NO: 39; and wherein each of the first plurality of peptides is shielded by a glycan, and each of the second plurality of peptides is not shielded by a glycan.

One aspect of the present disclosure provides a method for identifying a glycan-shielded conserved peptide of a glycoprotein, comprising: 1) determining and/or establishing a first 3D structure with a glycan profile and a second 3D structure without the glycan profile of the glycoprotein; 2) calculating a relative solvent accessibility (RSA) of an amino acid of the glycoprotein to identify a glycan-shielded amino acid that is exposed in the second 3D structure but shielded in the first 3D structure, based on the first 3D structure and the second 3D structure; 3) comparing amino acid sequences of a plurality of variants of the glycoprotein to identify a conserved sequence; and 4) mapping the result of the RSA calculation and the conserved sequence identified to identify a glycan-shielded conserved peptide, which comprises the conserved sequence with the glycan-shielded amino acid.

### BRIEF DESCRIPTION OF THE DRAWINGS

FIG. 1 is a graphic representation illustrating the domains and motifs of an exemplary spike protein with the annotations of N-glycosylation sites and O-glycosylation sites. The glycan-shielded conserved peptides identified in the present disclosure are also indicated.

FIG. 2 is an image of a Western Blot assay, showing the spike proteins expressed by host cells transfected with mRNAs configured to encode a Wuhan spike protein (WH S), a Delta spike protein (delta S), a modified Wuhan spike protein with deglycosylated S2 domain (WH S-(deg S2)), a modified Delta spike protein with deglycosylated S2 domain (delta S-(deg S2)), or a deglycosylated Wuhan spike protein (WH S-(deg S)), Anti-GADPH antibodies were used for positive control signals.

FIG. 3A is a graphic representation of IgG titers generated by a Wuhan spike protein (WH S), a Delta spike protein (delta S), a modified Wuhan spike protein with deglycosylated S2 domain (WH S-(deg-S2)), a modified Delta spike protein with deglycosylated S2 domain (delta S-(deg-S2)), or a deglycosylated Wuhan spike protein (WH S-(degS)) against a Wuhan spike protein (fully glycosylated).

FIG. 3B is a graphic representation of IgG titers generated by a Wuhan spike protein (WH S), a Delta spike protein (delta S), a modified Wuhan spike protein with deglycosylated S2 domain (WH S-(deg-S2)), a modified Delta spike protein with deglycosylated S2 domain (delta S-(deg-S2)), or a deglycosylated Wuhan spike protein (WH S-(degS)) against a deglycosylated Wuhan spike protein.

FIG. 3C is a graphic representation of IgG titers generated by a Wuhan spike protein (WH S), a Delta spike protein (delta S), a modified Wuhan spike protein with deglycosylated S2 domain (WH S-(deg-S2)), a modified Delta spike protein with deglycosylated S2 domain (delta S-(deg-S2)), or a deglycosylated Wuhan spike protein (WH S-(degS)) against a Delta spike protein (fully glycosylated).

FIG. 3D is a graphic representation of IgG titers generated by a Wuhan spike protein (WH S), a Delta spike protein (delta S), a modified Wuhan spike protein with deglycosylated S2 domain (WH S-(deg-S2)), a modified Delta spike protein with deglycosylated S2 domain (delta S-(deg-S2)), or a deglycosylated Wuhan spike protein (WH S-(degS)) against a deglycosylated Delta spike protein.

FIG. 3E is a graphic representation of IgG titers generated by a Wuhan spike protein (WH S) and a modified Wuhan spike protein with deglycosylated stem region (WH S-(deg-CD/HR2)) against a Wuhan spike protein (fully glycosylated).

FIG. 3F is a graphic representation of IgG titers generated by a Wuhan spike protein (WH S) and a modified Wuhan spike protein with deglycosylated stem region (WH S-(deg-CD/HR2)) against a Delta spike protein (fully glycosylated).

FIG. 3G is a graphic representation of IgG titers generated by a Wuhan spike protein (WH S) and a modified Wuhan spike protein with deglycosylated stem region (WH S-(deg-CD/HR2)) against an Omicron spike protein (fully glycosylated).

FIG. 4A is a graphic representation of the data of a neutralization assay, showing the neutralization activities of the vaccines designed to generate a Wuhan spike protein (WH S), a Delta spike protein (delta S), a modified Wuhan spike protein with deglycosylated S2 domain (WH S-(deg-S2)), and a modified Delta spike protein with deglycosylated S2 domain (delta S-(deg-S2)), respectively, against a Wuhan pseudovirus.

FIG. 4B is a graphic representation of the data of a neutralization assay, showing the neutralization activities of the vaccines designed to generate a Wuhan spike protein (WH S), a Delta spike protein (delta S), a modified Wuhan spike protein with deglycosylated S2 domain (WH S-(deg-S2)), and a modified Delta spike protein with deglycosylated S2 domain (delta S-(deg-S2)), respectively, against a Delta pseudovirus.

FIG. 4C is a graphic representation of the data of a neutralization assay, showing the neutralization activities of the vaccines designed to generate a Wuhan spike protein (WH S), a Delta spike protein (delta S), a modified Wuhan spike protein with deglycosylated S2 domain (WH S-(deg-S2)), and a modified Delta spike protein with deglycosylated S2 domain (delta S-(deg-S2)), respectively, against an Omicron BA.1 pseudovirus.

FIG. 4D is a graphic representation of the data of a neutralization assay, showing the neutralization activities of the vaccines designed to generate a Wuhan spike protein (WH S), a Delta spike protein (delta S), a modified Wuhan spike protein with deglycosylated S2 domain (WH S-(deg-S2)), and a modified Delta spike protein with deglycosylated S2 domain (delta S-(deg-S2)), respectively, against an Omicron BA.1.12.1 pseudovirus.

FIG. 4E is a graphic representation of the data of a neutralization assay, showing the neutralization activities of the vaccines designed to generate a Wuhan spike protein (WH S), a Delta spike protein (delta S), a modified Wuhan spike protein with deglycosylated S2 domain (WH S-(deg-S2)), and a modified Delta spike protein with deglycosylated S2 domain (delta S-(deg-S2)), respectively, against an Omicron BA.2 pseudovirus.

FIG. 4F is a graphic representation of the data of a neutralization assay, showing the neutralization activities of the vaccines designed to generate a Wuhan spike protein (WH S), a Delta spike protein (delta S), a modified Wuhan spike protein with deglycosylated S2 domain (WH S-(deg-S2)), and a modified Delta spike protein with deglycosylated S2 domain (delta S-(deg-S2)), respectively, against an Omicron BA.4/5 pseudovirus.

FIG. 5A is a graphic representation of the data of a GrzB ELISpot assay, showing the granzyme B (GrzB)-secreting T cells induced by the vaccines designed to generate a Wuhan spike protein (WH S), a Delta spike protein (delta S), a modified Wuhan spike protein with deglycosylated S2 domain (WH S-(deg-S2)), and a modified Delta spike protein with deglycosylated S2 domain (delta S-(deg-S2)), respectively. The T cells were isolated from immunized mice and incubated with the peptide pool of Wuhan spike proteins.

FIG. 5B is a graphic representation of the data of a GrzB ELISpot assay, showing the granzyme B (GrzB)-secreting T cells induced by the vaccines designed to generate a Wuhan spike protein (WH S), a Delta spike protein (delta S), a modified Wuhan spike protein with deglycosylated S2 domain (WH S-(deg-S2)), and a modified Delta spike protein with deglycosylated S2 domain (delta S-(deg-S2)), respectively. The T cells were isolated from immunized mice and incubated with the peptide pool of the RBD of the Wuhan spike proteins.

FIG. 5C is a graphic representation of the data of a GrzB ELISpot assay, showing the granzyme B (GrzB)-secreting T cells induced by the vaccines designed to generate a Wuhan spike protein (WH S), a Delta spike protein (delta S), a modified Wuhan spike protein with deglycosylated S2 domain (WH S-(deg-S2)), and a modified Delta spike protein with deglycosylated S2 domain (delta S-(deg-S2)), respectively. The T cells were isolated from immunized mice and incubated with the peptide pool of the S2 domain of the Wuhan spike proteins.

FIG. 6 is an image of a Western blot assay, showing the T cell response induced in HEK cells transfected to induce immune responses against a Delta spike protein (delta S) and a modified Delta spike protein with deglycosylated S2 domain (delta S-(deg-S2)), respectively. The cells were transfected and incubated with G-132 (MedChemExpress) or DMSO at 37° C. for 24 h.

FIG. 7A is a graphic representation of IgG titers generated by a Wuhan spike protein (WH S), a Delta spike protein (delta S), a modified Wuhan spike protein with deglycosylated S2 domain (WH S-(deg-S2)), a modified Delta spike protein with deglycosylated S2 domain (delta S-(deg-S2)), or a deglycosylated Wuhan spike protein (WH S-(degS)) against a human alpha coronavirus HCoV-NL63 spike protein (fully glycosylated).

FIG. 7B is a graphic representation of IgG titers generated by a Wuhan spike protein (WH S), a Delta spike protein (delta S), a modified Wuhan spike protein with deglycosylated S2 domain (WH S-(deg-S2)), a modified Delta spike protein with deglycosylated S2 domain (delta S-(deg-S2)), or a deglycosylated Wuhan spike protein (WH S-(degS)) against a human alpha coronavirus HCoV-229E spike protein (fully glycosylated).

FIG. 7C is a graphic representation of IgG titers generated by a Wuhan spike protein (WH S), a Delta spike protein (delta S), a modified Wuhan spike protein with deglycosylated S2 domain (WH S-(deg-S2)), a modified Delta spike protein with deglycosylated S2 domain (delta S-(deg-S2)), or a deglycosylated Wuhan spike protein (WH S-(degS)) against a human beta coronavirus HCoV-HKU1 spike protein (fully glycosylated).

FIG. 7D is a graphic representation of IgG titers generated by a Wuhan spike protein (WH S), a Delta spike protein (delta S), a modified Wuhan spike protein with deglycosylated S2 domain (WH S-(deg-S2)), a modified Delta spike protein with deglycosylated S2 domain (delta S-(deg-S2)), or a deglycosylated Wuhan spike protein (WH S-(degS)) against a human beta coronavirus HCoV-OC43 spike protein (fully glycosylated).

FIG. 7E is a graphic representation of IgG titers generated by a Wuhan spike protein (WH S), a Delta spike protein (delta S), a modified Wuhan spike protein with deglycosylated S2 domain (WH S-(deg-S2)), a modified Delta spike protein with deglycosylated S2 domain (delta S-(deg-S2)), or a deglycosylated Wuhan spike protein (WH S-(degS)) against a human beta coronavirus MERS-COV spike protein (fully glycosylated).

FIG. 7F is a graphic representation of IgG titers generated by a Wuhan spike protein (WH S), a Delta spike protein (delta S), a modified Wuhan spike protein with deglycosylated S2 domain (WH S-(deg-S2)), a modified Delta spike protein with deglycosylated S2 domain (delta S-(deg-S2)), or a deglycosylated Wuhan spike protein (WH S-(degS)) against a human beta coronavirus SARS-COV spike protein (fully glycosylated).

FIG. 8A is a graphic representation of the data of a neutralization assay, showing the neutralization activities of the vaccines designed to generate a Wuhan spike protein (WH S), a Delta spike protein (delta S), a modified Wuhan spike protein with deglycosylated S2 domain (WH S-(deg-S2)), and a modified Delta spike protein with deglycosylated S2 domain (delta S-(deg-S2)), respectively, against a MERS-COV pseudovirus.

FIG. 8B is a graphic representation of the data of a neutralization assay, showing the neutralization activities of the vaccines designed to generate a Wuhan spike protein (WH S), a Delta spike protein (delta S), a modified Wuhan spike protein with deglycosylated S2 domain (WH S-(deg-S2)), and a modified Delta spike protein with deglycosylated S2 domain (delta S-(deg-S2)), respectively, against a SARS-COV pseudovirus.

FIG. 9A provides a graphic representation of the FACS analysis results demonstrating the efficacy of the exemplary inventive embodiments. The experiments demonstrated the uptake of the novel nano-delivery formulations targeting BDMCs, according to embodiments of the present disclosure, compared to traditional LNPs. The FITC+ values shown in the figures are fluorescent intensities in arbitrary units (A.U.). FIG. 9A shows the BDMC cells uptake of a negative control group.

FIG. 9B provides a graphic representation of the FACS analysis results demonstrating the efficacy of the exemplary inventive embodiments. The experiments demonstrated the uptake of the novel nano-delivery formulations targeting BDMCs, according to embodiments of the present disclosure, compared to traditional LNPs. The FITC+ values shown in the figures are fluorescent intensities in arbitrary units (A.U.). FIG. 9B shows the non-BDMC cells uptake of a negative control group.

FIG. 9C provides a graphic representation of the FACS analysis results demonstrating the efficacy of the exemplary inventive embodiments. The experiments demonstrated the uptake of the novel nano-delivery formulations targeting BDMCs, according to embodiments of the present disclosure, compared to traditional LNPs. The FITC+ values shown in the figures are fluorescent intensities in arbitrary units (A.U.). FIG. 9C shows the BDMC cells uptake of a positive control group treated with FITC-labeled LNP.

FIG. 9D provides a graphic representation of the FACS analysis results demonstrating the efficacy of the exemplary inventive embodiments. The experiments demonstrated the uptake of the novel nano-delivery formulations targeting BDMCs, according to embodiments of the present disclosure, compared to traditional LNPs. The FITC+ values shown in the figures are fluorescent intensities in arbitrary units (A.U.). FIG. 9D shows the non-BDMC cells uptake of a positive control group treated with FITC-labeled LNP.

FIG. 9E provides a graphic representation of the FACS analysis results demonstrating the efficacy of the exemplary inventive embodiments. The experiments demonstrated the uptake of the novel nano-delivery formulations targeting BDMCs, according to embodiments of the present disclosure, compared to traditional LNPs. The FITC+ values shown in the figures are fluorescent intensities in arbitrary units (A.U.). FIG. 9E shows the BDMC cells uptake of an experiment group treated with FITC-labeled Compound 24-LNP according to an example of the present disclosure.

FIG. 9F provides a graphic representation of the FACS analysis results demonstrating the efficacy of the exemplary inventive embodiments. The experiments demonstrated the uptake of the novel nano-delivery formulations targeting BDMCs, according to embodiments of the present disclosure, compared to traditional LNPs. The FITC+ values shown in the figures are fluorescent intensities in arbitrary units (A.U.). FIG. 9F shows the non-BDMC cells uptake of a positive control group treated with FITC-labeled Compound 24-LNP according to an example of the present disclosure.

FIG. 9G provides a graphic representation of the FACS analysis results demonstrating the efficacy of the exemplary inventive embodiments. The experiments demonstrated the uptake of the novel nano-delivery formulations targeting BDMCs, according to embodiments of the present disclosure, compared to traditional LNPs. The FITC+ values shown in the figures are fluorescent intensities in arbitrary units (A.U.). FIG. 9G shows the BDMC cells uptake of an experiment group treated with FITC-labeled Compound 25-LNP according to an example of the present disclosure.

FIG. 9H provides a graphic representation of the FACS analysis results demonstrating the efficacy of the exemplary inventive embodiments. The experiments demonstrated the uptake of the novel nano-delivery formulations targeting BDMCs, according to embodiments of the present disclosure, compared to traditional LNPs. The FITC+ values shown in the figures are fluorescent intensities in arbitrary units (A.U.). FIG. 9H shows the non-BDMC cells uptake of a positive control group treated with FITC-labeled Compound 25-LNP according to an example of the present disclosure.

FIG. 10A provides a graphic representation of the FACS analysis results demonstrating the efficacy of the exemplary novel dendritic cell-targeting formulations of the present disclosure. This figure showed the results of non-LNP treated negative control group of dendritic cells (DC). The FITC+ values shown in the figures are fluorescent intensities in arbitrary units (A.U.).

FIG. 10B provides a graphic representation of the FACS analysis results demonstrating the efficacy of the exemplary novel dendritic cell-targeting formulations of the present disclosure. This figure showed the results of non-LNP treated negative control group of B cells. The FITC+ values shown in the figures are fluorescent intensities in arbitrary units (A.U.).

FIG. 10C provides a graphic representation of the FACS analysis results demonstrating the efficacy of the exemplary novel dendritic cell-targeting formulations of the present disclosure. This figure showed the results of non-LNP treated negative control group of T cells. The FITC+ values shown in the figures are fluorescent intensities in arbitrary units (A.U.).

FIG. 10D provides a graphic representation of the FACS analysis results demonstrating the efficacy of the exemplary novel dendritic cell-targeting formulations of the present disclosure. This figure showed the results of a positive control group of dendritic cells (DC) treated with FITC-labelled LNPs. The FITC+ values shown in the figures are fluorescent intensities in arbitrary units (A.U.).

FIG. 10E provides a graphic representation of the FACS analysis results demonstrating the efficacy of the exemplary novel dendritic cell-targeting formulations of the present disclosure. This figure showed the results of a positive control group of B cells treated with FITC-labelled LNPs. The FITC+ values shown in the figures are fluorescent intensities in arbitrary units (A.U.).

FIG. 10F provides a graphic representation of the FACS analysis results demonstrating the efficacy of the exemplary novel dendritic cell-targeting formulations of the present disclosure. This figure showed the results of a positive control group of T cells treated with FITC-labelled LNPs. The FITC+ values shown in the figures are fluorescent intensities in arbitrary units (A.U.).

FIG. 10G provides a graphic representation of the FACS analysis results demonstrating the efficacy of the exemplary novel dendritic cell-targeting formulations of the present disclosure. This figure showed dendritic cells (DC) uptake of the exemplary novel dendritic targeting formulations of Compound 24-LNPs according to embodiments of the present disclosure. The FITC+ values shown in the figures are fluorescent intensities in arbitrary units (A.U.).

FIG. 10H provides a graphic representation of the FACS analysis results demonstrating the efficacy of the exemplary novel dendritic cell-targeting formulations of the present disclosure. This figure showed B cells uptake of the exemplary novel dendritic targeting formulations of Compound 24-LNPs according to embodiments of the present disclosure. The FITC+ values shown in the figures are fluorescent intensities in arbitrary units (A.U.).

FIG. 10I provides a graphic representation of the FACS analysis results demonstrating the efficacy of the exemplary novel dendritic cell-targeting formulations of the present disclosure. This figure showed T cell uptake of the exemplary novel dendritic targeting formulations of Compound 24-LNPs according to embodiments of the present disclosure. The FITC+ values shown in the figures are fluorescent intensities in arbitrary units (A.U.).

FIG. 10J provides a graphic representation of the FACS analysis results demonstrating the efficacy of the exemplary novel dendritic cell-targeting formulations of the present disclosure. This figure showed dendritic cells (DC) uptake of the exemplary novel dendritic targeting formulations of Compound 25-LNPs according to embodiments of the present disclosure. The FITC+ values shown in the figures are fluorescent intensities in arbitrary units (A.U.).

FIG. 10K provides a graphic representation of the FACS analysis results demonstrating the efficacy of the exemplary novel dendritic cell-targeting formulations of the present disclosure. This figure showed B cells uptake of the exemplary novel dendritic targeting formulations of Compound 25-LNPs according to embodiments of the present disclosure. The FITC+ values shown in the figures are fluorescent intensities in arbitrary units (A.U.).

FIG. 10L provides a graphic representation of the FACS analysis results demonstrating the efficacy of the exemplary novel dendritic cell-targeting formulations of the present disclosure. This figure showed T cell uptake of the exemplary novel dendritic targeting formulations of Compound 25-LNPs according to embodiments of the present disclosure. The FITC+ values shown in the figures are fluorescent intensities in arbitrary units (A.U.).

FIG. 11A provides a graphic representation showing the FACS analysis results demonstrating the efficacy of the exemplary novel dendritic targeting formulations of the present disclosure. The experiments showed the uptake of the dendritic cell-targeting formulations according to embodiments of the present disclosure targeting BDMCs compared with conventional LNPs. The FITC+ values shown in the figures are fluorescent intensities in arbitrary units (A.U.). FIG. 11A showed a negative control with LNPs without using the present disclosure's novel targeting compound/formulation (i.e., “traditional LNP” as described herein).

FIG. 11B provides a graphic representation showing the FACS analysis results demonstrating the efficacy of the exemplary novel dendritic targeting formulations of the present disclosure. The experiments showed the uptake of the dendritic cell-targeting formulations according to embodiments of the present disclosure targeting BDMCs compared with conventional LNPs. FIG. 11B showed the experimental group treated with Compound 22-LNPs (22-LNP) comprising 5 mol % Compound 22. The FITC+ values shown in the figures are fluorescent intensities in arbitrary units (A.U.).

FIG. 11C provides a graphic representation showing the FACS analysis results demonstrating the efficacy of the exemplary novel dendritic targeting formulations of the present disclosure. The experiments showed the uptake of the dendritic cell-targeting formulations according to embodiments of the present disclosure targeting BDMCs compared with conventional LNPs. FIG. 11C showed the experimental group treated with Compound 22-LNPs (22-LNP) comprising 10 mol % Compound 22. The FITC+ values shown in the figures are fluorescent intensities in arbitrary units (A.U.).

FIG. 11D provides a graphic representation showing the FACS analysis results demonstrating the efficacy of the exemplary novel dendritic targeting formulations of the present disclosure. The experiments showed the uptake of the dendritic cell-targeting formulations according to embodiments of the present disclosure targeting BDMCs compared with conventional LNPs. FIG. 11D showed the experimental group treated with Compound 22-LNPs (22-LNP) comprising 20 mol % Compound 22. The FITC+ values shown in the figures are fluorescent intensities in arbitrary units (A.U.).

FIG. 11E provides a graphic representation showing the FACS analysis results demonstrating the efficacy of the exemplary novel dendritic targeting formulations of the present disclosure. The experiments showed the uptake of the dendritic cell-targeting formulations according to embodiments of the present disclosure targeting BDMCs compared with conventional LNPs. FIG. 11E showed the experimental group treated with Compound 23-LNPs (23-LNP) comprising 5 mol % Compound 23. The FITC+ values shown in the figures are fluorescent intensities in arbitrary units (A.U.).

FIG. 11F provides a graphic representation showing the FACS analysis results demonstrating the efficacy of the exemplary novel dendritic targeting formulations of the present disclosure. The experiments showed the uptake of the dendritic cell-targeting formulations according to embodiments of the present disclosure targeting BDMCs compared with conventional LNPs. FIG. 11F showed the experimental group treated with Compound 23-LNPs (23-LNP) comprising 10 mol % Compound 23. The FITC+ values shown in the figures are fluorescent intensities in arbitrary units (A.U.).

FIG. 11G provides a graphic representation showing the FACS analysis results demonstrating the efficacy of the exemplary novel dendritic targeting formulations of the present disclosure. The experiments showed the uptake of the dendritic cell-targeting formulations according to embodiments of the present disclosure targeting BDMCs compared with conventional LNPs. FIG. 11G showed the experimental group treated with Compound 23-LNPs (23-LNP) comprising 20 mol % Compound 23. The FITC+ values shown in the figures are fluorescent intensities in arbitrary units (A.U.).

FIG. 12A provides a graphic representation showing the FACS analysis results demonstrating the efficacy of the exemplary novel dendritic cell-targeting formulations of the present disclosure. The experiments showed the transfection of the targeting formulations BDMCs compared with LNPs without the compound of the present disclosure. The FITC+ values shown in the figures are fluorescent intensities in arbitrary units (A.U.). FIG. 12A showed a negative control with LNPs formed without using the present disclosure's novel targeting compound/formulation (i.e., “traditional LNP” as described herein).

FIG. 12B provides a graphic representation showing the FACS analysis results demonstrating the efficacy of the exemplary novel dendritic targeting formulations of the present disclosure. The experiments showed the transfection of the dendritic cell-targeting formulations according to embodiments of the present disclosure targeting BDMCs compared with conventional LNPs. FIG. 12B showed the experimental group treated with Compound 22-LNPs (22-LNP) comprising 5 mol % Compound 22. The FITC+ values shown in the figures are fluorescent intensities in arbitrary units (A.U.).

FIG. 12C provides a graphic representation showing the FACS analysis results demonstrating the efficacy of the exemplary novel dendritic targeting formulations of the present disclosure. The experiments showed the transfection of the dendritic cell-targeting formulations according to embodiments of the present disclosure targeting BDMCs compared with conventional LNPs. FIG. 12C showed the experimental group treated with Compound 22-LNPs (22-LNP) comprising 10 mol % Compound 22. The FITC+ values shown in the figures are fluorescent intensities in arbitrary units (A.U.).

FIG. 12D provides a graphic representation showing the FACS analysis results demonstrating the efficacy of the exemplary novel dendritic targeting formulations of the present disclosure. The experiments showed the transfection of the dendritic cell-targeting formulations according to embodiments of the present disclosure targeting BDMCs compared with conventional LNPs. FIG. 12D showed the experimental group treated with Compound 22-LNPs (22-LNP) comprising 20 mol % Compound 22. The FITC+ values shown in the figures are fluorescent intensities in arbitrary units (A.U.).

FIG. 12E provides a graphic representation showing the FACS analysis results demonstrating the efficacy of the exemplary novel dendritic targeting formulations of the present disclosure. The experiments showed the transfection of the dendritic cell-targeting formulations according to embodiments of the present disclosure targeting BDMCs compared with conventional LNPs. FIG. 12E showed the experimental group treated with Compound 23-LNPs (23-LNP) comprising 5 mol % Compound 23. The FITC+ values shown in the figures are fluorescent intensities in arbitrary units (A.U.).

FIG. 12F provides a graphic representation showing the FACS analysis results demonstrating the efficacy of the exemplary novel dendritic targeting formulations of the present disclosure. The experiments showed the transfection of the dendritic cell-targeting formulations according to embodiments of the present disclosure targeting BDMCs compared with conventional LNPs. FIG. 12F showed the experimental group treated with Compound 23-LNPs (23-LNP) comprising 10 mol % Compound 23. The FITC+ values shown in the figures are fluorescent intensities in arbitrary units (A.U.).

FIG. 12G provides a graphic representation showing the FACS analysis results demonstrating the efficacy of the exemplary novel dendritic targeting formulations of the present disclosure. The experiments showed the transfection of the dendritic cell-targeting formulations according to embodiments of the present disclosure targeting BDMCs compared with conventional LNPs. FIG. 12G showed the experimental group treated with Compound 23-LNPs (23-LNP) comprising 20 mol % Compound 23. The FITC+ values shown in the figures are fluorescent intensities in arbitrary units (A.U.).

FIG. 13A provides a graphic representation demonstrating the targeting efficacy and specificity of the exemplary formulation based on the distribution of the targeting LNPs in an animal model. The LNPs carried an mRNA configured to encode a luciferase in the targeted cells of the tested animals. The assay would generate detectable luminescence if the LNPs successfully transfect cells and the cells express the luciferase. The results clearly showed tissue-specific targeting of spleen and lymph tissue by the exemplary targeting formulation, thereby providing supporting evidence of immune cell (e.g., dendritic cell) specificity. FIG. 13A shows the result of a positive control LNP.

FIG. 13B provides a graphic representation demonstrating the targeting efficacy and specificity of the exemplary formulation based on the distribution of the targeting LNPs in an animal model. The LNPs carried an mRNA configured to encode a luciferase in the targeted cells of the tested animals. The assay would generate detectable luminescence if the LNPs successfully transfect cells and the cells express the luciferase. The results clearly showed tissue-specific targeting of spleen and lymph tissue by the exemplary targeting formulation, thereby providing supporting evidence of immune cell (e.g., dendritic cell) specificity. FIG. 13B shows the result of the Compound 22-LNP.

FIG. 13C provides a graphic representation demonstrating the targeting efficacy and specificity of the exemplary formulation based on the distribution of the targeting LNPs in an animal model. The LNPs carried an mRNA configured to encode a luciferase in the targeted cells of the tested animals. The assay would generate detectable luminescence if the LNPs successfully transfect cells and the cells express the luciferase. The results clearly showed tissue-specific targeting of spleen and lymph tissue by the exemplary targeting formulation, thereby providing supporting evidence of immune cell (e.g., dendritic cell) specificity. FIG. 13C shows the result of the Compound 12-LNP.

FIG. 14 shows the ¹H NMR spectrum of compound 12 of the present disclosure.

FIG. 15 shows the ¹³C NMR spectrum of compound 12 of the present disclosure.

FIG. 16A presents bar charts showing the IFNγ induction by the exemplary targeting LNPs formulation according to an embodiment of the present disclosure in vivo. The sera were collected from experimental animals 2 hours, 24 hours, and 48 hours after administration.

FIG. 16B presents bar charts showing the IL-4 induction by the exemplary targeting LNPs formulation according to an embodiment of the present disclosure in vivo. The sera were collected from experimental animals 2 hours, 24 hours, and 48 hours after administration.

FIG. 17 shows a graphic representation demonstrating the neutralization inhibitory effects of the exemplary LNPs of the present disclosure compared to control LNPs. The neutralization inhibition was evaluated against different dilution factors to show the difference between samples.

FIG. 18 provides a bar chart demonstrating that the exemplary LNPs, according to the present disclosure, carrying mRNA encoding wide-type spike protein, were able to invoke IgG production in vivo against wild-type virus and Delta and Omicron strains thereof.

FIG. 19 shows a comparative bar chart demonstrating the IgG titer (A) and neutralization ability (B) induced respectively by commercially available LNPs vs. LNP formulation formulated based on, and/or constructed with, exemplary compounds according to embodiments of the present disclosure. Both LNPs carried mRNA encoding a wide-type spike protein.

FIG. 20 shows the LCMS spectrum of compound 21 of the present disclosure.

### DETAILED DESCRIPTION OF THE INVENTION

Since the outbreak of Severe Acute Respiratory Syndrome Virus-2 (SARS-COV-2) in 2019, the virus has spread rapidly around the world and has continued to evolve with mutations. As of Feb. 15, 2023, the virus and its variants infected more than 650 million people and caused more than 6.6 million deaths. A great deal of global efforts has been directed toward developing effective strategies to contain the pandemic; among them, vaccination has been the most effective. Of the many vaccine candidates available, the successful development of mRNA vaccines represents a breakthrough in the field. After administration of an mRNA vaccine, it is translated in vivo to the corresponding protein antigen to elicit immune responses. Unlike adenovirus-type vaccines, which transduce mainly local tissues, mRNA vaccines provide broader biodistribution. Currently, all mRNA vaccines are developed based on the surface spike protein of the virus as an immunogen. However, various emerging variants, such as Delta and Omicron subvariants, can significantly escape the immune responses to the spike protein. Therefore, to develop a broadly protective vaccine against the current and upcoming variants, it is necessary to analyze the large number of SARS-COV-2 S protein sequences in the GISAID (Global Initiative on Sharing All Influenza Data) database, and identify the conserved sequences as targets for development of vaccines with broad protection and long-acting immunity.

### Glycan-Shielded Conserved Sequences of Spike Protein

Viruses are coated with host-made sugars/glycans to facilitate infection and to shield the conserved epitopes from immune response, and deletion of the glycan shields from spike protein exposed highly conserved epitopes and elicited broadly protective immune responses. Accordingly, one aspect of the present disclosure provides a method for identifying a glycan-shielded conserved peptide of a glycoprotein.

The method comprises determining and/or establishing a first 3-dimensional (3D) structure with a glycan profile and a second 3D structure without the glycan profile of the glycoprotein; calculating a relative solvent accessibility (RSA) of an amino acid of the glycoprotein to identify a glycan-shielded amino acid that is exposed in the second 3D structure but shielded in the first 3D structure based on the first 3D structure and the second 3D structure; comparing amino acid sequences of a plurality of variants of the glycoprotein to identify a conserved sequence; and mapping the result of the RSA calculation and the conserved sequence identified to identify a glycan-shielded conserved peptide, which comprises the conserved sequence comprising the glycan-shielded amino acid.

In some embodiments, the glycoprotein is a spike protein of virus. In certain embodiments, the virus is a coronavirus, an influenza virus, a human immunodeficiency virus, a human papillomavirus, a dengue virus, a Zika virus, an Epstein-Barr virus, a monkeypox virus, an Ebola virus, a Hepatitis B virus, or a Hepatitis C virus. Yet in some specific embodiments, a spike protein of a SARS-COV, MERS-COV, or SARS-COV-2 virus. In some embodiments, the plurality of variants of the glycoprotein comprises a SARS-COV-2 alpha variant, a SARS-COV-2 beta variant, a SARS-COV-2 delta variant, or a SARS-COV-2 omicron variant, meaning that the conserved sequence is a sequence, fragment (e.g., a peptide), region, motif, or domain of the spike protein that remain relatively unchanged across multiple variants.

It is important to note that the steps of the method of the present disclosure are not limited to the order as described above. For example, in one embodiment, the method first identifies any amino acid of the glycoprotein being a glycan-shielded amino acid and then identifies a conserved sequence to observe whether the conserved sequence comprises the glycan-shielded amino acid. In one example of such embodiments, every amino acid of the glycoprotein is to be distinguished as a glycan-shielded amino acid or not; yet in another example of such embodiments, every amino acid of a region of interest of the glycoprotein is to be distinguished as a glycan-shielded amino acid or not, wherein the region of interest can be a S2 domain or a stem region of a spike protein. In another embodiment, the conserved sequence can be identified first, and the amino acids constitute the conversed sequence can be distinguished as a glycan-shielded amino acid or not to identify whether the conserved sequence is a glycan-shielded conserved peptide.

In some embodiments, calculating a relative solvent accessibility (RSA) of an amino acid of the glycoprotein identifies a plurality of glycan-shielded amino acids, wherein one or more glycan-shielded amino acids of the plurality is within or constitutes a conserved sequence, while the rest of the plurality are not. In some embodiments, comparing amino acid sequences of a plurality of variants of the glycoprotein identifies a plurality of conserved sequences, wherein one or more conserved sequences of the plurality comprises one or more glycan-shielded amino acid, while others might not comprise any glycan-shielded amino acid.

In some embodiments, mapping the result of the RSA calculation and the conserved sequence identified to identify a glycan-shielded conserved peptide comprises observing whether the conserved sequence comprises one or more glycan-shielded amino acids. In certain embodiments, the 3D structures might be established based on a sequence of the glycoprotein different from the sequences used in identifying the conserved sequence. For example, the 3D structures might be established using a wild-type glycoprotein, but the conserved sequence was identified using sequences of variants. In such situation, it is possible that the numbering of the amino acids between the 3D structures and the conserved sequence does not match; therefore, mapping the result of the RSA calculation and the conserved sequence would comprise aligning the numbering system to identify the corresponding amino acids.

In some embodiments, the method further comprises identifying a glycosylation site of the glycoprotein. In some embodiments, the glycosylation site comprises a glycosylation sequon: N-Xa-S/T, wherein N denotes an asparagine (N) residue, S denotes a serine(S) residue, T denotes a threonine (T) residue, and Xa in the sequon is any amino acid residue except proline, and S/T denotes a serine or threonine residue. In some embodiments, the glycoprotein comprises at least one glycosylation site adjacent to the glycan-shielded conserved peptide thereof. As used herein, “adjacent to” describes that, in some situations, the glycosylation site is distanced from the glycan-shielded conserved peptide in 40 amino acids, upstream or downstream, or in other situations, the glycosylation site is not located in proximity on a linear peptide but is located close to the glycan-shielded conserved peptide in a folded form of the glycoprotein.

### 3D Structure Simulation.

In some embodiments, the first 3D structure and the second 3D structure can be constructed by using the amino acid sequence of the glycoprotein of any virus variant. For example, the amino acid sequence of the glycoprotein used to construct the first 3D structure and the second 3D structure can be from a SARS-COV-2 alpha variant, a SARS-COV-2 beta variant, a SARS-COV-2 delta variant, or a SARS-COV-2 omicron variant. In some specific embodiments, the amino acid sequence of the glycoprotein used to construct the first 3D structure and the second 3D structure is a spike protein of a SARS-COV-2 Wuhan strain or a SARS-COV-2 Delta strain. For example, the spike protein of the SARS-COV-2 Wuhan strain comprises an amino acid sequence at least 80%, 85%, 90%, 91%, 92%, 93%, 94%, 95%, 96%, 97%, 98%, or 99%, or 100% identical to the amino acid sequence set forth in SEQ ID NO: 01 or SEQ ID NO: 12.

In certain embodiments, the 3D structure of the glycoprotein can be established by using CHARMM-GUI (Lehigh University, Bethlehem) and OpenMM based on the Protein Data Bank (PDB) with the most abundant glycoform of BEAS-2B data as representative glycan profile. Furthermore, in some embodiments, establishing a first 3D structure with glycan profile and a second 3D structure without glycan profile further comprises determining the secondary structure of the glycoprotein. In certain embodiments, the protein secondary structure was determined by majority voting in the Dictionary of Secondary Structure of Proteins (DSSP) program and 2Struc web server. Nevertheless, the present disclosure is not limited to those bioinformatic platforms and databases. In some embodiments, the 3D structures used in the methods of the present disclosure can be obtained from existing databases.

### Relative Solvent Accessibility (RSA) and Glycan-Shielded Amino Acids.

Based on the established 3D structures, an amino acid of the glycoprotein is distinguished as being buried within the 3D structure, exposed, or shielded by the glycan. In some embodiments, for calculating the RSA, a probe radius is chosen with respect to a complementarity determining region (CDR)'s hypervariable loop of an antibody. In certain embodiments, the probe radius is about 5, 6, 7, 8, 9, 10, 11, 12, 13, 14, or 15 Angstrom (Å), or any ranges defined by the foregoing endpoints, such as 5 to 15 Angstrom, 5 to 14 Angstrom, 5 to 13 Angstrom, 5 to 12 Angstrom, 5 to 11 Angstrom, 5 to 10 Angstrom, 5 to 9 Angstrom, 6 to 15 Angstrom, 6 to 14 Angstrom, 6 to 13 Angstrom, 6 to 12 Angstrom, 6 to 11 Angstrom, 6 to 10 Angstrom, 6 to 9 Angstrom, 7 to 15 Angstrom, 7 to 14 Angstrom, 7 to 13 Angstrom, 7 to 12 Angstrom, 7 to 11 Angstrom, 7 to 10 Angstrom, or 7 to 9 Angstrom. In certain embodiments, amino acids with RSA above 3%, 4%, 5%, 6%, 7%, 8%, 9%, or 10% (which is considered as the extent to which a given amino acid in a protein structure is exposed to the solvent) were regarded as exposed, otherwise as buried. Based on that, glycans are considered to provide shielding for the residues with buried states in models with glycans, whereas these same residues have exposed states in models without glycans.

### Conserved Sequences.

In some embodiments that the glycoprotein is a spike protein of a SARS-COV-2 virus, the plurality of variants of the glycoprotein comprises a SARS-COV-2 alpha variant, a SARS-COV-2 beta variant, a SARS-COV-2 delta variant, or a SARS-COV-2 omicron variant, or a combination thereof. In certain embodiments, the variants might include Alpha strains (B.1.1.7), Beta strains (B.1.351), Gamma strains (P.1), Delta strains (B.1.617.2), Omicron strains (BA.1, BA.2, BA.3, BA.4, BA.5, BA.2.12.1, BA.2.75*), or a combination thereof. In yet certain embodiments, the variants might include one or more strains of BA.2.47, BQ.1, BQ.1.1, BQ.1.1.28, BQ.1.1.32, CH.1.1.3, EG.5.1, EL.1, EU.1.1, FD.1.1, JN.1, KP.2, KP.3, XBB.1.16, XBB.1.16.6, XBB.1.17.1, XBB.1.5, XBB.1.5.10, XBB.1.5.59, XBB.1.9.1, XBB.1.9.2, XBB.2.3, XBB.2.3.3, XBB.2.3.8, and XBF, based on GISAID (version: Feb. 8, 2023, Apr. 24, 2023, Jun. 13, 2023, and Aug. 19, 2023).

In some embodiments, the conserved sequence comprises about 10, 11, 12, 13, 14, 15, 16, 17, 18, 19, 20, 21, 22, 23, 24, 25, 26, 27, 28, 29, or 30 amino acids, or any ranges defined by the foregoing endpoints, such as 10 to 30 amino acids, 10 to 25 amino acids, 10 to 20 amino acids, 12 to 30 amino acids, 12 to 25 amino acids, 12 to 20 amino acids, 15 to 30 amino acids, 15 to 25 amino acids, or 15 to 20 amino acids. In some embodiments, a conserved sequence is a sequence having a mutation rate no higher than 5%, 3%, 1%, 0.5%, or 0.1%, or any ranges defined by the foregoing endpoints, such as 5% to 0.1%, 3% to 0.1%, 1% to 0.1%, 0.5% to 0.1%, 5% to 0.5%, 5% to 1%, 3% to 0.5%, or 3% to 1%. In certain embodiments, a conserved sequence is a sequence having a mutation rate no higher than 1% and is not affected by mutations in a dominant strain or in a top-ranked emerging variant based on the GISAID database.

### Glycan-Shielded Conserved Peptide.

A glycan-shielded conserved peptide, as described in the present disclosure, does not necessarily mean that the conserved peptide is completely shielded by glycan. In some embodiments, the glycan-shielded conserved peptide can be partially shielded by the glycan. As used herein, “glycan-shielded” describes that the peptide is structurally shielded, completely or partially, or the peptide's interaction with a host immune system is interfered, completely or partially, by the glycan. In other words, the glycan shielding the peptide interferes the peptide from being presented to a host immune system as an antigen.

In certain embodiments, at least 10%, 20%, 30%, 40%, 50%, 60%, 70%, 80%, 90%, or 95%, or 100% of the amino acids of the conserved sequence are glycan-shielded amino acid, or any ranges defined by the foregoing endpoints, such as 10% to 100%, 10% to 95%, 10% to 90%, 10% to 80%, 10% to 70%, 10% to 60%, 10% to 50%, 10% to 40%, 10% to 30%, 30% to 100%, 30% to 95%, 30% to 90%, 30% to 80%, 30% to 70%, 30% to 60%, 30% to 50%, 30% to 40%, 50% to 100%, 50% to 95%, 50% to 90%, 50% to 80%, 50% to 70%, 50% to 60%, 70% to 100%, 70% to 95%, 70% to 90%, or 70% to 80%.

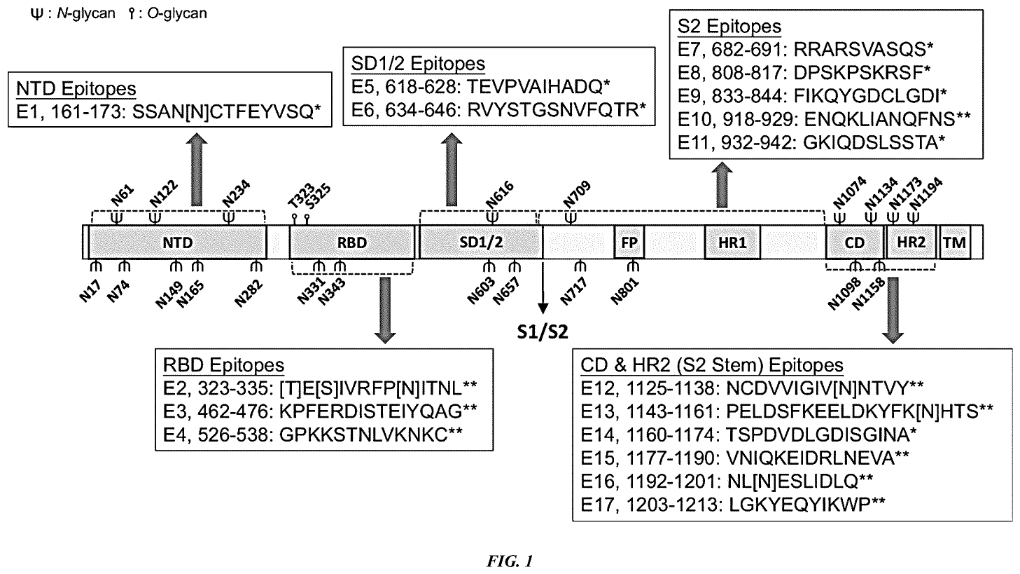

In some embodiments wherein the glycoprotein is a spike protein (S protein) of SARS-COV-2 virus; the present disclosure analyzed 14 million S protein sequences reported to GISAID and identified 17 glycan-shielded conserved peptides (see Table below and FIG. 1) on the viral surface, including those with less than 1% residue mutation rates. Of these 17 conserved epitopes, 11 are in the S2 domain, including 6 in the CD/HR2 (stem) region.

TABLE
Glycan-shielded conserved peptides

| SEQ ID NO. | Sequences | Location |
|---|---|---|
| SEQ ID NO: 23 | SSANNCTFEY | S1 |
| | VSQ | |
| SEQ ID NO: 24 | TESIVREPNI | S1 |
| | TNL | |
| SEQ ID NO: 25 | KPFERDISTE | S1 |
| | IYQAG | |
| SEQ ID NO: 26 | GPKKSTNLVK | S1 |
| | NKC | |
| SEQ ID NO: 27 | TEVPVAIHAD | S1 |
| | Q | |
| SEQ ID NO: 28 | RVYSTGSNVF | S1 |
| | QTR | |
| SEQ ID NO: 29 | RRARSVASQS | S2 |
| SEQ ID NO: 30 | DPSKPSKRSF | S2 |
| SEQ ID NO: 31 | FIKQYGDCLG | S2 |
| | DI | |
| SEQ ID NO: 32 | ENQKLIANQF | S2 |
| | NS | |
| SEQ ID NO: 33 | GKIQDSLSST | S2 |
| | A | |
| SEQ ID NO: 34 | NCDVVIGIVN | S2 |
| | NTVY | (stem) |
| SEQ ID NO: 35 | PELDSFKEEL | S2 |
| | DKYFKNHTS | (stem) |
| SEQ ID NO: 36 | TSPDVDLGDI | S2 |
| | SGINA | (stem) |
| SEQ ID NO: 37 | VNIQKEIDRL | S2 |
| | NEVA | (stem) |
| SEQ ID NO: 38 | NLNESLIDLQ | S2 |
| | | (stem) |
| SEQ ID NO: 39 | LGKYEQYIKW | S2 |
| | P | (stem) |

The present disclosure, in those embodiments, also found that the six linear peptides in the stem are the most conserved and shielded by the glycans from the six N-glycosites in this region. The other conserved peptides are spread in different domains. Based on this finding and previous experience, it is believed that deletion of the glycan shields in the stem or the S2 domain will expose the highly conserved epitopes and elicit broadly protective immune responses.

### Immunogenic Peptide

One aspect of the present disclosure also provides an isolated immunogenic peptide. The immunogenic peptide comprises at least one amino acid sequence selected from a group consisting of: SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, and SEQ ID NO: 39. Without wishing to be bound by theories, each of the sequences of SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, and SEQ ID NO: 39 represents a glycan-shielded conserved peptide. Because those peptides are shielded by glycans in nature, one can appreciate that those peptides are rarely exposed to a host immune system, so they are hardly considered immunogenic before the present disclosure. As the present disclosure teaches to removing the glycosylation site, thereby removing the glycan shield covering those peptides, the experiments of the present disclosure observed the immune response and cross-activities induced by those peptides, proving those exposed peptides are immunogenic (e.g., epitopes that are recognized by host immune systems).

As described herein, “isolated” means that a subject protein or polypeptide (1) is free of at least some other proteins or polypeptides with which it would typically be found in nature, (2) is essentially free of other proteins or polypeptides from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a protein or polypeptide with which the “isolated protein” or “isolated polypeptide” may be associated in nature, (6) is operably associated (by covalent or noncovalent interaction) with a polypeptide with which it is not associated in nature, or (7) does not occur in nature. Such an isolated protein or polypeptide can be encoded by genomic DNA, cDNA, mRNA or other RNA, of may be of synthetic origin according to any of a number of well-known chemistries for artificial peptide and protein synthesis, or any combination thereof. In certain embodiments, the isolated protein or polypeptide is substantially free from proteins or polypeptides or other contaminants that are found in its natural environment that would interfere with its use (therapeutic, diagnostic, prophylactic, research or otherwise).

In some examples, the isolated immunogenic peptide can be synthesized and engineered into a synthetic framework so that it can be presented in a same or similar 3D structure as it was in its naturally occurring protein to induce an immune response; however, the synthetic framework is a different protein from the naturally occurring protein in, for example, their amino acid sequences or in their structures. In a specific example, the synthetic framework is different from a spike protein from which the immunogenic peptide is derived from, therefore, an isolated immunogenic peptide is different from a peptide of the same sequences existing in nature. In certain embodiments, the immunogenic peptide might be engineered into a synthetic framework, and the resulting protein might comprise an amino acid sequence at least 80%, 85%, 90%, 91%, 92%, 93%, 94%, 95%, 96%, 97%, 98%, or 99%, or 100% identical to SEQ ID NO: 08, provided that each one of the Q12, Q36, Q46, Q72, Q96, and Q111 is not Asn (N) residue. In yet certain embodiments, the immunogenic peptide might be engineered into a synthetic framework, and the resulting protein might comprise an amino acid sequence at least 80%, 85%, 90%, 91%, 92%, 93%, 94%, 95%, 96%, 97%, 98%, or 99%, or 100% identical to SEQ ID NO: 19, provided that each one of the Q12, Q36, Q46, Q72, Q96, and Q111 is not Asn (N) residue.

In some embodiments, the isolated immunogenic peptide comprises at least one amino acid sequence selected from a group consisting of: SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, and SEQ ID NO: 39. In certain embodiments, the isolated immunogenic peptide comprises at least one amino acid sequence selected from a group consisting of SEQ ID NO: 29, SEQ ID NO: 30, SEQ ID NO: 31, SEQ ID NO: 32, and SEQ ID NO: 33. In certain embodiments, the isolated immunogenic peptide comprises at least one amino acid sequence selected from a group consisting of SEQ ID NO: 34, SEQ ID NO: 35, SEQ ID NO: 36, SEQ ID NO: 37, SEQ ID NO: 38, and SEQ ID NO: 39.

### Broad Spectrum Vaccines of Low-Sugar Spike Protein

To develop a broadly protective vaccine against the current and upcoming variants, one aspect of the present disclosure provides a nucleic acid, encoding a recombinant spike protein, wherein the recombinant spike protein is glycosylated, provided that a stem region thereof is devoid of a N-linked glycosylation site. The present disclosure provides that although deletion of the glycan shields of a spike protein can expose the highly conserved epitopes and elicit broadly protective immune responses, glycosylation is also critical for protein folding. Thus, removing glycosylation sites on a glycoprotein can significantly affect the folding of the glycoprotein. As a result, the resulting glycoprotein can be structurally different from the glycoprotein with the original glycan profile, and while the glycoprotein is configured to induce an immune response against a virus (i.e., as a vaccine), the efficacy of the induced immune response against the virus could be demolished.

Therefore, without wishing to be bound by theories, the present disclosure provides that removing the glycosylation sites of the spike protein can significantly affect the folding of the spike protein, resulting in a spike protein that is not able to induce protection against the infection of the SARS-COV-2 virus. In comparison, if only the glycosylation sites in the S2 domain or the stem region of the spike protein are removed, the spike protein can still fold properly, and the resulting spike protein can still provide protection against the infection of the SARS-COV-2 virus, while most importantly, provides broad protection against various variants. The present disclosure further provides that the stem region, especially in a Wuhan strain backbone or a Delta strain backbone, is the key region to remove the glycan shield for obtaining a broad cross-activity while not demolishing the protein structure significantly.

Accordingly, the present disclosure contemplates that a spike protein, which remains glycosylated, provided that a stem region thereof is devoid of an N-linked glycosylation site. The glycosylation site of the spike protein can be located in the S1 domain and/or the S2 domain thereof. As the recombinant spike protein of the present disclosure has less glycosylation than a wild-type spike protein, it is considered as “low-sugar” spike protein, and a vaccine designed to express such a spike protein is referred to as a low-sugar vaccine.

In the embodiments that the spike protein comprises an N-linked glycosylation site in the S1 domain, the N-linked glycosylation site can be located in the receptor binding domain (RBD) and/or the N-terminal domain (NTD). In the embodiments that the spike protein comprises an N-linked glycosylation site in the S2 domain, the N-linked glycosylation site can be located outside of the stem region as the stem region of the spike protein, according to the present disclosure, is devoid of an N-linked glycosylation site.

As described herein, “devoid of an N-linked glycosylation site” describes that the stem region of the recombinant spike protein does not have any N-linked glycosylation site. The N-linked glycosylation site, in some embodiments, comprises a glycosylation sequon: N-Xa-S/T, wherein Xa in the sequon is any amino acid residue except proline, and S/T denotes a serine or threonine residue. In some embodiments, the nucleic acid of the present disclosure can be viewed as a nucleic acid modified from or derived from a reference nucleic acid encoding a reference spike protein having a glycosylation site in the stem region thereof. In such embodiments, compared with the reference spike protein, the spike protein of the present disclosure does not have a glycosylation sequon in the stem region thereof, or it has a disrupted glycosylation sequon where the asparagine (Asn; N) residue is replaced with another amino acid (e.g., a glutamine (Glu; Q) residue) so a glycan cannot attach.

### Wuhan Strain Spike Protein as the Reference

In some embodiments, the reference spike protein is a spike protein of a SARS-CoV-2 Wuhan strain. In certain embodiments, the reference spike protein comprises an amino acid sequence at least 80%, 85%, 90%, 91%, 92%, 93%, 94%, 95%, 96%, 97%, 98%, or 99%, or 100% identical to SEQ ID NO: 01.

S2-deg. In certain embodiments, the spike protein comprises a S2 domain, comprising an amino acid sequence at least 80%, 85%, 90%, 91%, 92%, 93%, 94%, 95%, 96%, 97%, 98%, or 99%, or 100% identical to SEQ ID NO: 05 (as shown below), provided that at least one of the X23, X31, X115, X388, X412, X422, X448, X472, and X487 is not an Asn (N) residue. In some embodiments, each of the X23, X31, X115, X388, X412, X422, X448, X472, and X487 is not an N residue. In some embodiments, at least one of X23, X31, X115, X388, X412, X422, X448, X472, and X487 is an Gln (Q) residue. In some embodiments, each of the X23, X31, X115, X388, X412, X422, X448, X472, and X487 is an Q residue.

In certain embodiment, the spike protein comprises a S2 domain, comprising an amino acid sequence at least 80%, 85%, 90%, 91%, 92%, 93%, 94%, 95%, 96%, 97%, 98%, or 99%, or 100% identical to SEQ ID NO: 06, provided that each of the Q23, Q31, Q115, Q388, Q412, Q422, Q448, Q472, and Q487 is not an N residue.

SEQ ID NO: 05

VASQ SIIAYTMSLG AENSVAYSXN SIAIPTXFTI
SVTTEILPVS MTKTSVDCTM YICGDSTECS NLLLQYGSFC
TQLNRALTGI AVEQDKNTQE VFAQVKQIYK TPPIKDEGGF
XFSQILPDPS KPSKRSFIED LLENKVTLAD AGFIKQYGDC
LGDIAARDLI CAQKENGLTV LPPLLTDEMI AQYTSALLAG
TITSGWTFGA GAALQIPFAM QMAYRENGIG VTQNVLYENQ
KLIANQENSA IGKIQDSLSS TASALGKLQD VVNQNAQALN
TLVKQLSSNF GAISSVLNDI LSRLDKVEAE VQIDRLITGR
LQSLQTYVTQ QLIRAAEIRA SANLAATKMS ECVLGQSKRV
DFCGKGYHLM SFPQSAPHGV VELHVTYVPA QEKXFTTAPA
ICHDGKAHFP REGVFVSXGT HWFVTQRXFY EPQIITTDNT
FVSGNCDVVI GIVXNTVYDP LQPELDSFKE ELDKYFKXHT
SPDVDLGDIS GIXASVVNIQ KEIDRLNEVA KNLNESLIDL
QELGKYEQYI KWPWYIWLGF IAGLIAIVMV TIMLCCMTSC
CSCLKGCCSC GSCCKEDEDD SEPVLKGVKL HYT

In certain embodiments, the spike protein of the present invention comprises an amino acid sequence at least 80%, 85%, 90%, 91%, 92%, 93%, 94%, 95%, 96%, 97%, 98%, or 99%, or 100% identical to SEQ ID NO: 02, provided that at least one of X17, X61, X74, X122, X149, X165, X234, X282, X331, X343, X394, X487, X603, X616, and X657 is an Asn (N) residue, and at least one of X709, X717, X801, X1074, X1098, X1108, X1134, X1158, and X1173 is not an N residue. In certain embodiments, the spike protein of the present invention comprises an amino acid sequence at least 80%, 85%, 90%, 91%, 92%, 93%, 94%, 95%, 96%, 97%, 98%, or 99%, or 100% identical to SEQ ID NO: 02, provided that each of the X17, X61, X74, X122, X149, X165, X234, X282, X331, X343, X394, X487, X603, X616, and X657 is an Asn (N) residue, and at least one of the X709, X717, X801, X1074, X1098, X1108, X1134, X1158, and X1173 is not an N residue. In certain embodiments, the spike protein of the present invention comprises an amino acid sequence at least 80%, 85%, 90%, 91%, 92%, 93%, 94%, 95%, 96%, 97%, 98%, or 99%, or 100% identical to SEQ ID NO: 02, provided that each of the X17, X61, X74, X122, X149, X165, X234, X282, X331, X343, X394, X487, X603, X616, and X657 is an N residue, and each of the X709, X717, X801, X1074, X1098, X1108, X1134, X1158, and X1173 is not an N residue (e.g., is an Q residue).

In certain embodiments, the spike protein of the present invention comprises an amino acid sequence at least 80%, 85%, 90%, 91%, 92%, 93%, 94%, 95%, 96%, 97%, 98%, or 99%, or 100% identical to SEQ ID NO: 03, provided that each of Q709, Q717, Q801, Q1074, Q1098, Q1108, Q1134, Q1158, and Q1173 is an Q residue. In some embodiments, the nucleic acid of the present disclosure comprises a nucleotide sequence at least 80%, 85%, 90%, 91%, 92%, 93%, 94%, 95%, 96%, 97%, 98%, or 99%, or 100% identical to SEQ ID NO: 10.

Stem-deg. In certain embodiments, the S2 domain comprises an amino acid sequence at least 80%, 85%, 90%, 91%, 92%, 93%, 94%, 95%, 96%, 97%, 98%, or 99%, or 100% identical to SEQ ID NO: 05, provided that each one of the X388, X412, X422, X448, X472, and X487 is not an Asn (N) residue. In certain embodiments, at least one of the X23, X31, and X115 is an Asn (N) residue, or all of them are N residues. In certain embodiments, the spike protein comprises a stem region, comprising an amino acid sequence at least 80%, 85%, 90%, 91%, 92%, 93%, 94%, 95%, 96%, 97%, 98%, or 99%, or 100% identical to SEQ ID NO: 07 (as shown below), provided at least one of the X12, X36, X46, X72, X96, and X111 is not an Asn (N) residue. In certain embodiments, each one of the X12, X36, X46, X72, X96, and X111 is not an N residue. In some embodiments, at least one of the X12, X36, X46, X72, X96, and X111 is an Gln (Q) residue. In certain embodiments, each one of the X12, X36, X46, X72, X96, and X111 is an Q residue. In some embodiments, the spike protein comprises a stem region, comprising an amino acid sequence at least 80%, 85%, 90%, 91%, 92%, 93%, 94%, 95%, 96%, 97%, 98%, or 99%, or 100% identical to SEQ ID NO: 08, provided that each one of the Q12, Q36, Q46, Q72, Q96, and Q111 is an Q residue.

SEQ Id NO: 07

LHVTYVPA QEKXFTTAPA ICHDGKAHFP REGVFVSXGT
HWFVTQRXFY EPQIITTDNT FVSGNCDVVI GIVXNTVYDP
LQPELDSFKE ELDKYFKXHT SPDVDLGDIS GIXASVVNIQ
KEIDRLNEVA KNLNESLIDL QELGKYEQYI K

In certain embodiments, the spike protein of the present invention comprises an amino acid sequence at least 80%, 85%, 90%, 91%, 92%, 93%, 94%, 95%, 96%, 97%, 98%, or 99%, or 100% identical to SEQ ID NO: 02, provided that at least one of X17, X61, X74, X122, X149, X165, X234, X282, X331, X343, X394, X487, X603, X616, X657, X709, X717, and X801 is an N residue, and at least one of X1074, X1098, X1108, X1134, X1158, and X1173 is not an N residue. In certain embodiments, each one of the X17, X61, X74, X122, X149, X165, X234, X282, X331, X343, X394, X487, X603, X616, X657, X709, X717, and X801 is an N residue, and at least one of X1074, X1098, X1108, X1134, X1158, and X1173 is not an N residue. Yet in certain embodiments, each one of the X17, X61, X74, X122, X149, X165, X234, X282, X331, X343, X394, X487, X603, X616, X657, X709, X717, and X801 is an N residue, and each one of the X1074, X1098, X1108, X1134, X1158, and X1173 is not an N residue (e.g., is an Q residue). In certain embodiments, the spike protein of the present invention comprises an amino acid sequence at least 80%, 85%, 90%, 91%, 92%, 93%, 94%, 95%, 96%, 97%, 98%, or 99%, or 100% identical to SEQ ID NO: 04, provided that each one of the Q1074, Q1098, Q1108, Q1134, Q1158, and Q1173 is not an N residue. In some embodiments, the nucleic acid of the present disclosure comprises a nucleotide sequence at least 80%, 85%, 90%, 91%, 92%, 93%, 94%, 95%, 96%, 97%, 98%, or 99%, or 100% identical to SEQ ID NO: 11.

### Delta Strain Spike Protein as the Reference

In some embodiments, the reference spike protein is a spike protein of a SARS-CoV-2 Delta strain. In certain embodiments, the reference spike protein comprises an amino acid sequence at least 80%, 85%, 90%, 91%, 92%, 93%, 94%, 95%, 96%, 97%, 98%, or 99%, or 100% identical to SEQ ID NO: 12.

S2-deg. In certain embodiments, the spike protein comprises a S2 domain, comprising an amino acid sequence at least 80%, 85%, 90%, 91%, 92%, 93%, 94%, 95%, 96%, 97%, 98%, or 99%, or 100% identical to SEQ ID NO: 16 (as shown below), provided that at least one of X23, X31, X115, X388, X412, X422, X448, X472, and X487 is not an N residue. In some embodiments, each of the X23, X31, X115, X388, X412, X422, X448, X472, and X487 is not an N residue. In some embodiments, at least one of X23, X31, X115, X388, X412, X422, X448, X472, and X487 is an Q residue. In some embodiments, each of the X23, X31, X115, X388, X412, X422, X448, X472, and X487 is an Q residue. In certain embodiment, the spike protein comprises a S2 domain, comprising an amino acid sequence at least 80%, 85%, 90%, 91%, 92%, 93%, 94%, 95%, 96%, 97%, 98%, or 99%, or 100% identical to SEQ ID NO: 17, provided that each of the Q23, Q31, Q115, Q388, Q412, Q422, Q448, Q472, and Q487 is not an N residue.

SEQ ID NO: 16

VASQSI IAYTMSLGAE NSVAYSXNSI AIPTXFTISV
TTEILPVSMT KTSVDCTMYI CGDSTECSNL LLQYGSFCTQ
LNRALTGIAV EQDKNTQEVE AQVKQIYKTP PIKDFGGEXF
SQILPDPSKP SKRSFIEDLL ENKVTLADAG FIKQYGDCLG
DIAARDLICA QKENGLTVLP PLLTDEMIAQ YTSALLAGTI
TSGWTFGAGA ALQIPFAMQM AYRENGIGVT QNVLYENQKL
IANQENSAIG KIQDSLSSTA SALGKLQNVV NQNAQALNTL
VKQLSSNEGA ISSVLNDILS RLDKVEAEVQ IDRLITGRLQ
SLQTYVTQQL IRAAEIRASA NLAATKMSEC VLGQSKRVDF
CGKGYHLMSF PQSAPHGVVF LHVTYVPAQE KXFTTAPAIC
HDGKAHFPRE GVFVSXGTHW FVTQRXFYEP QIITTDNTFV
SGNCDVVIGI VXNTVYDPLQ PELDSFKEEL DKYFKXHTSP
DVDLGDISGI XASVVNIQKE IDRLNEVAKN LNESLIDLQE
LGKYEQYIKW PWYIWLGFIA GLIAIVMVTI MLCCMTSCCS
CLKGCCSCGS CCKEDEDDSE PVLKGVKLHY T

In certain embodiments, the spike protein of the present invention comprises an amino acid sequence at least 80%, 85%, 90%, 91%, 92%, 93%, 94%, 95%, 96%, 97%, 98%, or 99%, or 100% identical to SEQ ID NO: 13, provided that at least one of X61, X74, X122, X149, X163, X232, X280, X329, X341, X392, X485, X601, X614, and X655 is an N residue, and at least one of X707, X715, X799, X1072, X1096, X1106, X1132, X1156, and X1171 is not an N residue. In certain embodiments, the spike protein of the present invention comprises an amino acid sequence at least 80%, 85%, 90%, 91%, 92%, 93%, 94%, 95%, 96%, 97%, 98%, or 99%, or 100% identical to SEQ ID NO: 13, provided that each of X61, X74, X122, X149, X163, X232, X280, X329, X341, X392, X485, X601, X614, and X655 is an N residue, and at least one of X707, X715, X799, X1072, X1096, X1106, X1132, X1156, and X1171 is not an N residue. In certain embodiments, the spike protein of the present invention comprises an amino acid sequence at least 80%, 85%, 90%, 91%, 92%, 93%, 94%, 95%, 96%, 97%, 98%, or 99%, or 100% identical to SEQ ID NO: 13, provided that each of X61, X74, X122, X149, X163, X232, X280, X329, X341, X392, X485, X601, X614, and X655 is an N residue, and each of X707, X715, X799, X1072, X1096, X1106, X1132, X1156, and X1171 is not an N residue (e.g., is an Q residue).

In certain embodiments, the spike protein of the present invention comprises an amino acid sequence at least 80%, 85%, 90%, 91%, 92%, 93%, 94%, 95%, 96%, 97%, 98%, or 99%, or 100% identical to SEQ ID NO: 14, provided that each of Q707, Q715, Q799, Q1072, Q1096, Q1106, Q1132, Q1156, and Q1171 is an Q residue. In some embodiments, the nucleic acid of the present disclosure comprises a nucleotide sequence at least 80%, 85%, 90%, 91%, 92%, 93%, 94%, 95%, 96%, 97%, 98%, or 99%, or 100% identical to SEQ ID NO: 21.

Stem-deg. In certain embodiments, the S2 domain comprises an amino acid sequence at least 80%, 85%, 90%, 91%, 92%, 93%, 94%, 95%, 96%, 97%, 98%, or 99%, or 100% identical to SEQ ID NO: 16, provided that each one of the X388, X412, X422, X448, X472, and X487 is not an Asn (N) residue. In certain embodiments, at least one of the X23, X31, and X115 is an Asn (N) residue, or all of them are N residues. In certain embodiments, the spike protein comprises a stem region, comprising an amino acid sequence at least 80%, 85%, 90%, 91%, 92%, 93%, 94%, 95%, 96%, 97%, 98%, or 99%, or 100% identical to SEQ ID NO: 18 (as shown below), provided at least one of the X12, X36, X46, X72, X96, and X111 is not an N residue. In certain embodiments, each one of the X12, X36, X46, X72, X96, and X111 is not an N residue. In some embodiments, at least one of the X12, X36, X46, X72, X96, and X111 is an Q residue. In certain embodiments, each one of the X12, X36, X46, X72, X96, and X111 is an Q residue. In some embodiments, the spike protein comprises a stem region, comprising an amino acid sequence at least 80%, 85%, 90%, 91%, 92%, 93%, 94%, 95%, 96%, 97%, 98%, or 99%, or 100% identical to SEQ ID NO: 19, provided that each one of the Q12, Q36, Q46, Q72, Q96, and Q111 is not an N residue.

SEQ ID NO: 18

LHVTYVPAQE KXFTTAPAIC HDGKAHFPRE GVFVSXGTHW
FVTQRXFYEP QIITTDNTFV SGNCDVVIGI VXNTVYDPLQ
PELDSFKEEL DKYFKXHTSP DVDLGDISGI XASVVNIQKE
IDRLNEVAKN LNESLIDLQE LGKYEQYIK

In certain embodiments, the spike protein of the present invention comprises an amino acid sequence at least 80%, 85%, 90%, 91%, 92%, 93%, 94%, 95%, 96%, 97%, 98%, or 99%, or 100% identical to SEQ ID NO: 13, provided that at least one of X61, X74, X122, X149, X163, X232, X280, X329, X341, X392, X485, X601, X614, X655, X707, X715, and X799 is an Asn (N) residue, and at least one of X1072, X1096, X1106, X1132, X1156, and X1171 is not an N residue. In certain embodiments, each one of the X61, X74, X122, X149, X163, X232, X280, X329, X341, X392, X485, X601, X614, X655, X707, X715, and X799 is an N residue, and at least one of X1072, X1096, X1106, X1132, X1156, and X1171 is not an N residue. Yet in certain embodiments, each one of the X61, X74, X122, X149, X163, X232, X280, X329, X341, X392, X485, X601, X614, X655, X707, X715, and X799 is an Asu (N) residue, and each one of the X1072, X1096, X1106, X1132, X1156, and X1171 is not an Asu (N) residue (e.g., is an Q residue). In certain embodiments, the spike protein of the present invention comprises an amino acid sequence at least 80%, 85%, 90%, 91%, 92%, 93%, 94%, 95%, 96%, 97%, 98%, or 99%, or 100% identical to SEQ ID NO: 15, provided that each one of the Q1072, Q1096, Q1106, Q1132, Q1156, and Q1171 is not an Asu (N) residue. In some embodiments, the nucleic acid of the present disclosure comprises a nucleotide sequence at least 80%, 85%, 90%, 91%, 92%, 93%, 94%, 95%, 96%, 97%, 98%, or 99%, or 100% identical to SEQ ID NO: 22.

### Recombinant Spike Protein

The present disclosure also teaches a recombinant spike protein, which is derived from a SARS-COV-2 Wuhan strain or a SARS-COV-2 Delta strain.

In one aspect of Wuhan strain, given that the spike protein is glycosylated but has a S2 domain devoid of an N-glycosylation site, the recombinant spike protein comprises an amino acid sequence at least 80%, 85%, 90%, 91%, 92%, 93%, 94%, 95%, 96%, 97%, 98%, or 99%, or 100% identical to SEQ ID NO: 02, wherein X denotes any amino acid, provided that at least one of the X17, X61, X74, X122, X149, X165, X234, X282, X331, X343, X394, X487, X603, X616, and X657 is an Asn (N) residue, and at least one of the X709, X717, X801, X1074, X1098, X1108, X1134, X1158, and X1173 is not an Asn (N) residue. In some embodiments, each one of the X709, X717, X801, X1074, X1098, X1108, X1134, X1158, and X1173 is not an Asn (N) residue

In another aspect of Wuhan strain, given that the spike protein is glycosylated but has a stem region devoid of an N-glycosylation site, the recombinant spike protein comprises an amino acid sequence at least 80%, 85%, 90%, 91%, 92%, 93%, 94%, 95%, 96%, 97%, 98%, or 99%, or 100% identical to SEQ ID NO: 02, wherein X denotes any amino acid, provided that at least one of the X17, X61, X74, X122, X149, X165, X234, X282, X331, X343, X394, X487, X603, X616, X657, X709, X717, and X801 is an Asn (N) N residue, and at least one of X1074, X1098, X1108, X1134, X1158, and X1173 is not an Asn (N) N residue. In some embodiments, each one of X1074, X1098, X1108, X1134, X1158, and X1173 is not an Asn (N) N residue.

In another aspect of Wuhan strain, given that the spike protein may or may not be glycosylated but has a S2 domain devoid of an N-glycosylation site, the recombinant spike protein comprises an amino acid sequence at least 80%, 85%, 90%, 91%, 92%, 93%, 94%, 95%, 96%, 97%, 98%, or 99%, or 100% identical to SEQ ID NO: 05, wherein X denotes any amino acid, provided that at least one of the X23, X31, X115, X388, X412, X422, X448, X472, and X487 is not an Asn (N) residue. In some embodiments, each one of the X23, X31, X115, X388, X412, X422, X448, X472, and X487 is not an Asn (N) residue.

In another aspect of Wuhan strain, given that the spike protein may or may not be glycosylated but has a stem region devoid of an N-glycosylation site, the recombinant spike protein comprises an amino acid sequence at least 80%, 85%, 90%, 91%, 92%, 93%, 94%, 95%, 96%, 97%, 98%, or 99%, or 100% identical to SEQ ID NO: 07, wherein X denotes any amino acid, provided that at least one of the X12, X36, X46, X72, X96, and X111 is not an Asn (N) residue. In some embodiments, each one of the X12, X36, X46, X72, X96, and X111 is not an Asn (N) residue.

In one aspect of Delta strain, given that the spike protein is glycosylated but has a S2 domain devoid of an N-glycosylation site, the recombinant spike protein comprises an amino acid sequence at least 80%, 85%, 90%, 91%, 92%, 93%, 94%, 95%, 96%, 97%, 98%, or 99%, or 100% identical to SEQ ID NO: 13, wherein X denotes any amino acid, provided that at least one of the X61, X74, X122, X149, X163, X232, X280, X329, X341, X392, X485, X601, X614, and X655 is an Asn (N) residue, and at least one of X707, X715, X799, X1072, X1096, X1106, X1132, X1156, and X1171 is not an Asn (N) residue. In some embodiments, each one of the X707, X715, X799, X1072, X1096, X1106, X1132, X1156, and X1171 is not an Asn (N) residue.

In another aspect of Delta strain, given that the spike protein is glycosylated but has a stem region devoid of an N-glycosylation site, the recombinant spike protein comprises an amino acid sequence at least 80%, 85%, 90%, 91%, 92%, 93%, 94%, 95%, 96%, 97%, 98%, or 99%, or 100% identical to SEQ ID NO: 13, wherein X denotes any amino acid, provided that at least one of the X61, X74, X122, X149, X163, X232, X280, X329, X341, X392, X485, X601, X614, X655, X707, X715, and X799 is an Asn (N) residue, and at least one of the X1072, X1096, X1106, X1132, X1156, and X1171 is not an Asn (N) residue. In some embodiments, each one of the X1072, X1096, X1106, X1132, X1156, and X1171 is not an Asn (N) residue.

In another aspect of Delta strain, given that the spike protein may or may not be glycosylated but has a S2 domain devoid of an N-glycosylation site, the recombinant spike protein comprises an amino acid sequence at least 80%, 85%, 90%, 91%, 92%, 93%, 94%, 95%, 96%, 97%, 98%, or 99%, or 100% identical to SEQ ID NO: 16, wherein X denotes any amino acid, provided that at least one of the X23, X31, X115, X388, X412, X422, X448, X472, and X487 is not an Asn (N) residue. In some embodiments, each one of the X23, X31, X115, X388, X412, X422, X448, X472, and X487 is not an Asn (N) residue.

In another aspect of Delta strain, given that the spike protein may or may not be glycosylated but has a stem region devoid of an N-glycosylation site, the recombinant spike protein comprises an amino acid sequence at least 80%, 85%, 90%, 91%, 92%, 93%, 94%, 95%, 96%, 97%, 98%, or 99%, or 100% identical to SEQ ID NO: 18, wherein X denotes any amino acid, provided that at least one of the X12, X36, X46, X72, X96, and X111 is not an Asn (N) residue. In some embodiments, each one of the X12, X36, X46, X72, X96, and X111 is not an Asn (N) residue.

In yet another aspect, the present disclosure provides a recombinant spike protein, comprising a first plurality of peptides, each comprises an amino acid sequence selected from a group consisting of SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33; and a second plurality of peptides, each comprises an amino acid sequence selected from a group consisting of SEQ ID NO: 34, SEQ ID NO: 35, SEQ ID NO: 36, SEQ ID NO: 37, SEQ ID NO: 38, and SEQ ID NO: 39; and wherein each of the first plurality of peptides is shielded by a glycan, and each of the second plurality of peptides is not shielded by a glycan.

Without wishing to be bound by theories, the recombinant spike protein of the present disclosure is different from its naturally occurring counterpart at least in the glycol form thereof. A naturally occurring counterpart while being produced by a host cell will be glycosylated resulting in all the peptide described above including SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, and SEQ ID NO: 39 are all shielded by glycans.

In some embodiments, whether a peptide is shielded by a glycan can be determined by establishing a first 3D structure with glycan profile and a second 3D structure without glycan profile of the glycoprotein; and calculating a relative solvent accessibility (RSA) of an amino acid of the peptide to identify a glycan-shielded amino acid that is exposed in the second 3D structure but shielded in the first 3D structure, based on the first 3D structure and the second 3D structure. A peptide is considered shielded if at least 10%, 20%, 30%, 40%, 50%, 60%, 70%, 80%, 90%, or 95%, or 100% of the amino acids of the peptide are glycan-shielded amino acids. The 3D structure and the RSA calculation can be obtained as described herein.

### Vector and Immunogenic Composition

In one aspect, the present disclosure provides an expression vector comprising a nucleic acid of the present disclosure. In some embodiments, the expression vector is a lipid nanoparticle (LNP), a liposome, a polymersome, a viral particle, a plasmid, or a bead. In some embodiments, the nucleic acid is DNA. In some embodiments, the nucleic acid is RNA, such as a messenger RNA (mRNA) designed to encode the recombinant influenza HA according to an exemplary embodiment of the present disclosure.

In the embodiments that the nucleic acid is a mRNA designed to encode the recombinant spike protein in vivo, the nucleic acid can be further modified to improve its stability and translation capacity in a host cell. For example, in some embodiments, the nucleic acid further comprises a promoter, which can be recognized effectively by the ribosome of the host cells. In some embodiments, the nucleic acid further comprises a 5′ untranslated region (UTR), a 3′ UTR, or both to increase the stability and regulate the translation of the mRNA. In some embodiments, the nucleic acid further comprises a poly-A tail, which helps regulate the stability of the mRNA. In yet some embodiments, the nucleic acid further comprises a 5′ cap, which is important for recruiting translation initiation factors.

Furthermore, except for the additional elements described above, the mRNA configured to encode the recombinant spike protein according to an exemplary embodiment of the present disclosure can have its sequences modified. For example, in some embodiments, by codon optimization, the mRNA can be modified to use frequent codons, which enhances stability and translation. In some embodiments, codon optimization can be performed to modify the secondary structure of the mRNA. For example, the uridines of the mRNA might be replaced with 1-methyl-pseudouridine, which can effectively minimize the innate immune response to foreign mRNA, thereby enhancing the stability and translation of the mRNA in host cells.

### Synthesis of the Nucleic Acid

The nucleic acid of the present disclosure can be prepared using in vitro translation following conventional methods in the field. In some embodiments, a gene encoding a wild-type spike protein or a reference spike protein can be cloned into a conventional plasmid. Plasmids are used in the synthesis because they are easy to replicate and can reliably contain the target gene sequence. Genetic engineering approaches can be performed to modify the wild-type gene so that the N residue of the sequon N-X-S/T is substituted or to replace certain nucleotides for codon optimization. In some embodiments, the modified gene (i.e., a nucleic acid according to one exemplary embodiment of the present disclosure) can be cloned to an in vitro transcription (IVT) plasmid and flanked by a 5′UTR and a 3′UTR followed by a poly-A tail. The IVT plasmid can be reacted with a polymerase and treated with DNases to remove linear DNA. The product of the reaction can then, in some embodiments, react with capping enzymes, including Faustovirus Capping Enzyme (FCE) or Vaccinia Capping Enzyme (VCE) and mRNA cap 2′-O-methyltransferase, to obtain mRNA molecules ready for use. However, the present disclosure is not limited to the general synthesis methods described above or exemplified herein. The procedures for synthesizing an exemplary nucleic acid of the present disclosure can be as those described in Chaudhary, N. et al., mRNA vaccines for infectious diseases: principles, delivery and clinical translation. Nat Rev Drug Discov 20, 817-838 (2021), which is incorporated herein by reference in its entirety.

### Lipid Nanoparticle

In some embodiments, the expression vector is a lipid nanoparticle (LNP). LNPs are the leading delivery system used for mRNA vaccines. Conventional LNPs usually have four major components: a neutral phospholipid, cholesterol, a polyethylene-glycol (PEG)-lipid, and an ionizable cationic lipid. An exemplary LNP suitable for the present disclosure consists of SM-102 (heptadecan-9-yl 8-((2-hydroxyethyl) (6-oxo-6-(undecyloxy) hexyl) amino) octanoate), PEG2000-DMG (1-monomethoxypolyethyleneglycol-2,3-dimyristylglycerol with polyethylene glycol of average molecular weight 2000), 1,2-Distearoyl-sn-glycero-3 phosphocholine (DSPC), and cholesterol at a 50:10:38.5:1.5 ratio. Another exemplary LNP suitable for the present disclosure consists of ALC-0315 ((4-hydroxybutyl) azanediyl)bis(hexane-6,1-diyl)bis(2-hexyldecanoate)), ALC-0159 (2-[(polyethylene glycol)-2000]—N,N ditetradecylacetamide), 1,2-Distearoyl-sn-glycero-3-phosphocholine (DSPC), and cholesterol at a 46.3:9.4:42.7:1.6 ratio.

In some embodiments, the LNP further comprises a compound as described in PCT Application No. PCT/US24/23590, filed on Apr. 8, 2024, titled “METHODS AND COMPOSITIONS FOR DENDRITIC CELL TARGETING NANO-DELIVERY,” which is hereby incorporated by reference in its entirety. The compound might comprise:

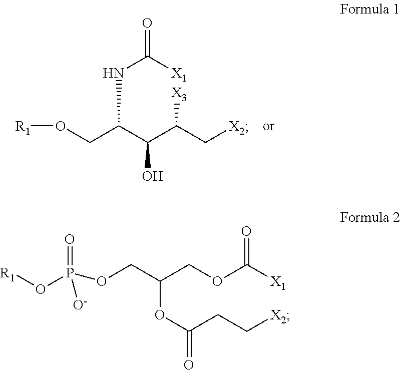

wherein R¹ comprises a substituted or non-substituted glycosyl group; wherein X₁ and X₂ are each independently hydrogen, alkyl, alkenyl, alkynyl, aryloxy, or a substituted version thereof, or —(CH₂)nX₄, n is 0 to 50, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N; and wherein X₃ is hydrogen, C₁₋₆ alkyl, or hydroxyl.

### R₁ Group.

Targeting Functionality. In certain embodiments, the R₁ group is configured to provide selective delivery or targeted delivery functionality for the exemplary LNP formulation formed by the component of the present disclosure. In some embodiments, the R₁ group is configured to target an antigen-presenting cell (e.g., a dendritic cell). In some embodiments, the target cell can be other types of immune cells. In yet some other embodiments, the target can be any biological cells where the payload is designed. In certain embodiments, the R₁ group is designed to have a targeting moiety, which can be a ligand of a receipt on a target cell. For example, the R₁ group might be configured to target the DC-SIGN of a dendritic cell.

Without wishing to be bound by theory, it is believed that mannoside and fucoside can bind a dendritic cell (e.g., via binding to DC-SIGN) with specificity. Therefore, in some embodiments, the R₁ group comprises a mannoside, fucoside, or both as the targeting moiety. The mannoside and/or the fucoside can be a terminal mannose or a terminal fucoside of the R₁ group, which might provide better chances to interact with a dendritic cell.

In some other embodiments, the R₁ group is configured to target Siglec-1, so the glycosyl group can comprise 9-N-(4H-thieno[3,2-c]chromene-2-carbamoyl)-Neu5Ac-α2,3-Gal-GlcNAc. In some embodiments, the R₁ group is configured to target Siglec-2, and the glycosyl group can comprise 9-Biphenyl Neu5Ac-α2,6-Gal-GlcNAc. In some embodiments, the R₁ group is configured to target Siglec-5/E, and the glycosyl group can comprise Neu5Ac-α2,3-Gal-GlcNAc.

In some embodiments, the R₁ group comprises a formula of R₂—R_{A}—, wherein R₂ is the substituted or non-substituted glycosyl group, and R_{A} is an attachment group, and wherein the attachment group is an aryl, an alkyl, an amide, an alkyl amide, a combination thereof, or a covalent bond. In some embodiments, the aryl comprises 0 to 3 substituents (e.g., 1 to 3 substituents), wherein the substituent of the aryl is C₁₋₆ alkyl, halide, or C₁₋₆ alkyl halide. In some embodiments, the attachment group is configured to provide structural flexibilities and/or facilitate the binding between the targeting moiety and the target. In certain embodiments, R₂ is conjugated covalently to R_{A} at a carbon of the glycosyl group, resulting in an O-glycosylation.

Binding in acidic conditions. In some embodiments, the binding between the glycosyl group of R₁ and a target is Ca²⁺-correlated, and the calcium coordination might decrease at a low pH environment, resulting in lower binding affinity. Therefore, to provide a better binding affinity under acidic conditions, the attachment group can comprise an aryl group. Without wishing to be bound by any theories, the aryl group may engage in the CH-π and hydrophobic interactions that enhance the binding under acidic conditions. The aryl group can be an unsubstituted benzene or a benzene substituted with a halide or an alkyl halide (e.g., a CF₃). In some embodiments, the aryl group is coupled with the targeting moiety. For example, the R₁ group can comprise an O-aryl mannoside.

Spacer. In some embodiments, the attachment group of R₁ comprises a spacer. The spacer is configured to provide structural flexibility to R₁. Without wishing to be bound by theories, the flexibility allows the glycosyl group of R₁ to move during the interaction between the targeting moiety and the target, thereby facilitating the binding between them.

In certain embodiments, a preferred spacer is biocompatible. In some embodiments, the initiator spacer comprises a saturated carbon moiety, a polyethylene glycol (PEG) moiety, or a combination thereof. For example, the spacer can be a polyethylene glycol (PEG) moiety, formed by 1, 2, 3, 4, 5, 6, 7, 8, 9, 10, 12, 15, 18, 20, 24, 30, 36, 40, 48, 50, 55, 60, 65, or 72 (OCH₂CH₂) subunits, or any ranges defined by the foregoing endpoints, such as 2 to 72, 2 to 60, 2 to 48, 2 to 36, 2 to 24, 2 to 18, 2 to 15, 2 to 10, 4 to 72, 4 to 60, 4 to 48, 4 to 36, 4 to 24, 4 to 18, 4 to 15, 4 to 10, 8 to 72, 8 to 60, 8 to 48, 8 to 36, 8 to 24, 8 to 18, 8 to 15, or 8 to 10 (OCH₂CH₂) subunits. In some embodiments, the PEG moiety can be a linear, branched, or star structure.

Structural configuration. In certain embodiments, the glycosyl group can be a linear structure or a branched structure. In some embodiments, the glycosyl group might have a plurality of targeting moieties, for example, 2, 3, 4, 5, 6, 7, 8, 9, or 10 targeting moieties. The plurality of targeting moieties can be arranged in a linear, branched, or star configuration. For example, the glycosyl group might comprise a mono-mannoside, a di-mannoside, or a tri-mannoside, and when the glycosyl group comprises a tri-mannoside, the tri-mannoside can be a linear form or a branched structure, such as a α-1,3-α-1,6-trimannoside. In certain embodiments, it is noticed that a branched configuration (e.g., a tri-mannoside glycan head) shows superior binding affinity to its target receptor.

In some embodiments, the R₁ group is a substituted glycosyl group. The glycosyl group might comprise 1 to 6 substituents, and each substituent can be C₁₋₆ alkyl, C₁₋₆ alkenyl, halogen, C₁₋₆ alkyl halide, C₁₋₆ alkoxy, amine, nitro, C₁₋₆ alkyl amine, amide, azido, aryl, cycloalkyl, heterocycloalkyl, sulfite, or a substituted version thereof, or a combination thereof. In certain embodiments, the substituent is conjugated to a carbon of the glycosyl group directly or is conjugated to the carbon via an O-yl conjugation (e.g., by replacing the hydrogen of the hydroxyl group on the carbon).

In certain embodiments, the substituent of the glycosyl group is selected from the group consisting of aryl, 5-membered cycloalkyl, 6-membered cycloalkyl, 5-membered heterocycloalkyl, and 6-membered heterocycloalkyl, and a substituted version thereof, which comprises 1 to 6 substituents selected from the group consisting of C₁₋₆ alkyl, halogen, C₁₋₆ alkyl halide, C₁₋₆ alkoxy, amine, nitro, C₁₋₆ alkyl amine, azido, amide, carboxyl, hydroxyl, aryl, cycloalkyl, heterocycloalkyl, or a substituted version thereof, or a combination thereof. In some embodiments, the heterocycloalkyl comprises 1 to 3 heteroatoms, selected from the group consisting of O, S, and N.

In some embodiments, the substituent of the glycosyl group is a substituted or non-substituted aryl, for example, a substituted or non-substituted phenyl group. In certain embodiments, the aryl is substituted with 1 to 6 substituents, each is independently selected from the group consisting of C₁₋₆ alkyl, halogen, C₁₋₆ alkyl halide, C₁₋₆ alkoxy, amine, nitro, C₁₋₆ alkyl amine, azido, amide, carboxyl, hydroxyl, aryl, cycloalkyl, heterocycloalkyl, or a substituted version thereof, or a combination thereof. In certain embodiments, the substituent of the glycosyl group is a phenyl (benzene ring) substituted with OH, CH₃, NH₂, CF₃, OCH₃, F, Br, Cl, NO₂, N₃, or a combination thereof. For example, the substituted benzene ring can be a phenol group.

In some embodiments, the R₁ group is a mono-mannoside substituted with 1 to 6 substituents, and each substituent can be C₁₋₆ alkyl, C₁₋₆ alkenyl, halogen, C₁₋₆ alkyl halide, amine, C₁₋₆ alkyl amine, amide, aryl, cycloalkyl, heterocycloalkyl, sulfite, or a substituted version thereof, or a combination thereof. In certain embodiments, the R₁ group is a mono-mannoside substituted with a first substitute and a second substitute; each of the first substitute and the second substitute is independently selected from a group consisting of C₁₋₆ alkyl, C₁₋₆ alkenyl, halogen, C₁₋₆ alkyl halide, amine, C₁₋₆ alkyl amine, amide, aryl, cycloalkyl, heterocycloalkyl, and sulfite.

In some embodiments, the R₁ group comprises a first mannoside and a second mannoside. Each of the first mannoside and the second mannoside is independently substituted with 1 to 6 substituents, and each substituent can be C₁₋₆ alkyl, C₁₋₆ alkenyl, halogen, C₁₋₆ alkyl halide, amine, C₁₋₆ alkyl amine, amide, aryl, cycloalkyl, heterocycloalkyl, sulfite, or a substituted version thereof, or a combination thereof.

Binding affinity. In some embodiments, the binding affinity between the glycosyl group of R₁ and a target can be defined by a dissociation constant (K_{D}). In some embodiments, the K_{D} at pH 7.4 can be 5, 10, 15, 20, 30, 40, 50, 75, 100, 125, 150, 200, 250, 300, 350, 400, 450, 500, 600, 700, 800, 900, 1000, 1250, 1500, 1750, 2000, 2250, 2500, 3000, 3250, 3500, 3750, 4000, 4250, 4500, 4750, 5000, 5250, 5500, 5750, 6000, 6250, 6500, 6750, 7000, 7250, 7500, 7750, or 8000 nM, or any range defined by the foregoing endpoints, such as, 5 to 8000, 5 to 7000, 5 to 6000, 5 to 5000, 5 to 4000, 5 to 3000, 5 to 2500, 5 to 2000, 5 to 1500, 5 to 1250, 5 to 1000, 5 to 900, 5 to 800, 5 to 700, 5 to 600, 5 to 500, 5 to 400, 5 to 300, 5 to 200, 5 to 150, 5 to 100, 5 to 75, 5 to 50, 5 to 30, 5 to 20, 10 to 8000, 10 to 7000, 10 to 6000, 10 to 5000, 10 to 4000, 10 to 3000, 10 to 2500, 10 to 2000, 10 to 1500, 10 to 1250, 10 to 1000, 10 to 900, 10 to 800, 10 to 700, 10 to 600, 10 to 500, 10 to 400, 10 to 300, 10 to 200, 10 to 150, 10 to 100, 10 to 75, 10 to 50, 10 to 30, or 10 to 20 nM.

In some other embodiments, the Kp at pH 5 can be 1, 2, 3, 4, 5, 10, 15, 20, 30, 40, 50, 75, 100, 125, 150, 200, 250, 300, 350, 400, 450, 500, 550, 600, 650, 700, 750, 800, 850, 900, 1000, 1250, 1500, 1750, or 2000 nM, or any range defined by the foregoing endpoints, such as, 1 to 2000, 1 to 1500, 1 to 1000, 1 to 900, 1 to 800, 1 to 750, 1 to 700, 1 to 650, 1 to 600, 1 to 550, 1 to 500, 1 to 450, 1 to 400, 1 to 350, 1 to 300, 1 to 250, 1 to 200, 1 to 150, 1 to 100, 1 to 75, 1 to 50, 1 to 40, 1 to 30, 1 to 20, 1 to 10, or to 5, 5 to 2000, 5 to 1500, 5 to 1000, 5 to 900, 5 to 800, 5 to 750, 5 to 700, 5 to 650, 5 to 600, 5 to 550, 5 to 500, 5 to 450, 5 to 400, 5 to 350, 5 to 300, 5 to 250, 5 to 200, 5 to 150, 5 to 100, 5 to 75, 5 to 50, 5 to 40, 5 to 30, 5 to 20, 5 to 10 nM.

Examples. In some embodiments, the R₁ group is selected from the group consisting of (each structure shown below is independent from one another despite whether it is separated using a semicolon with an adjacent structure):

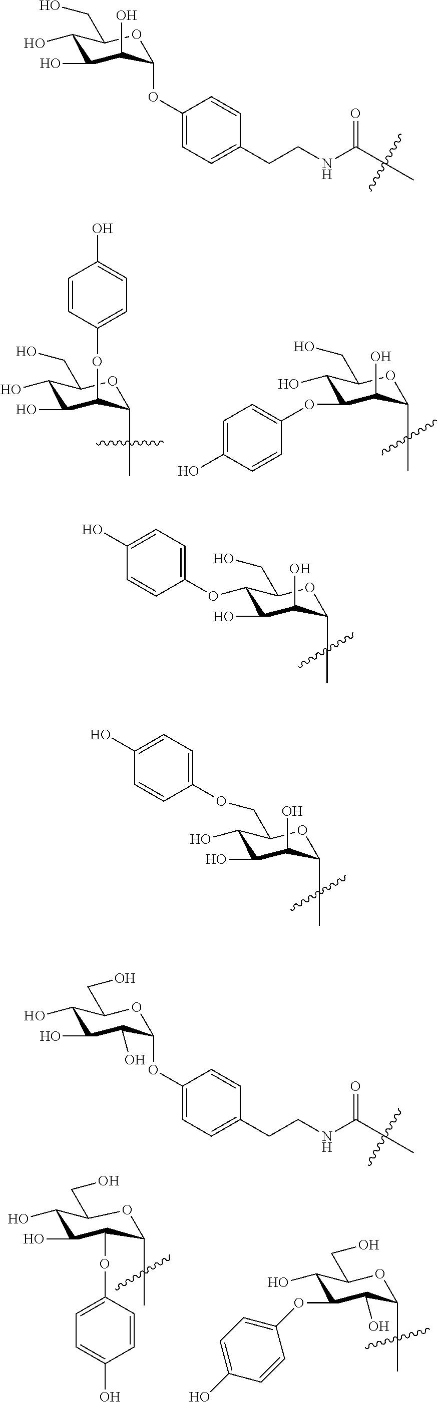

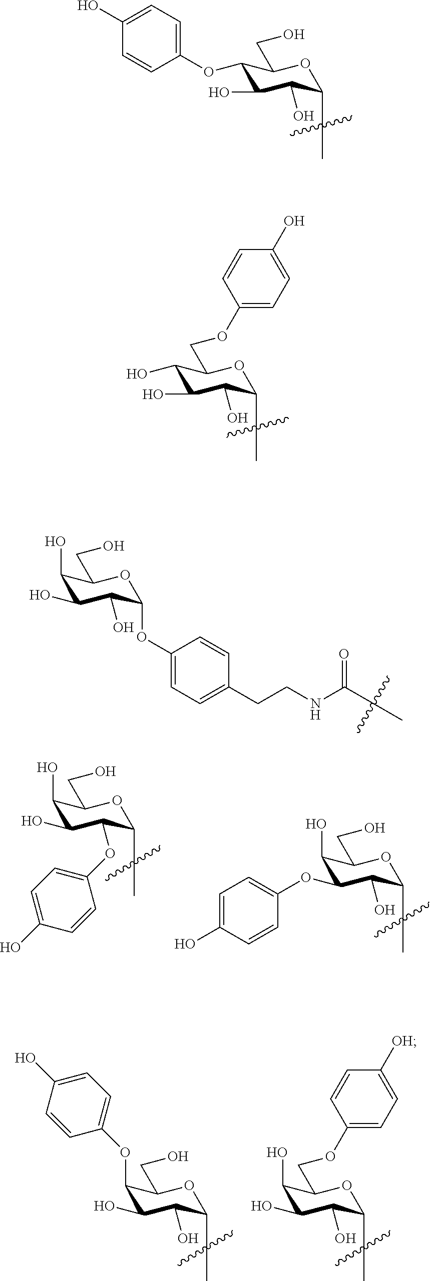

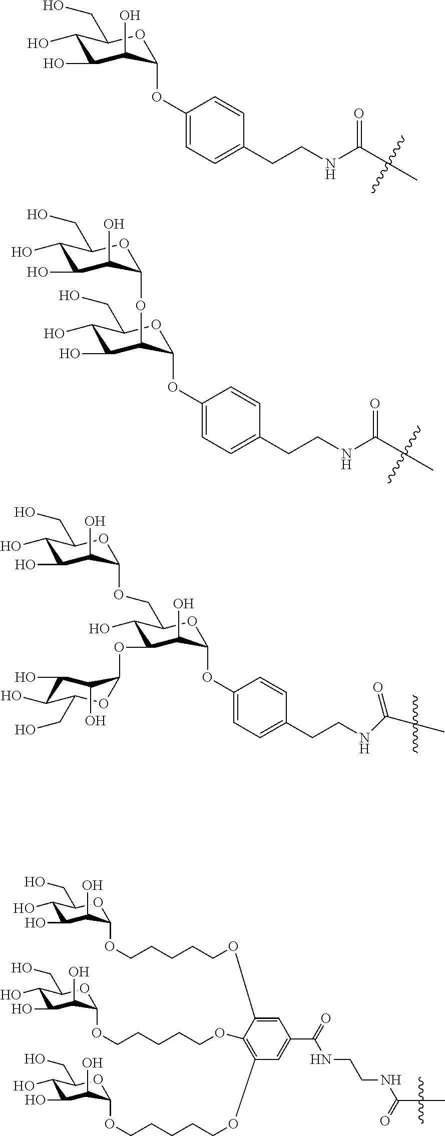

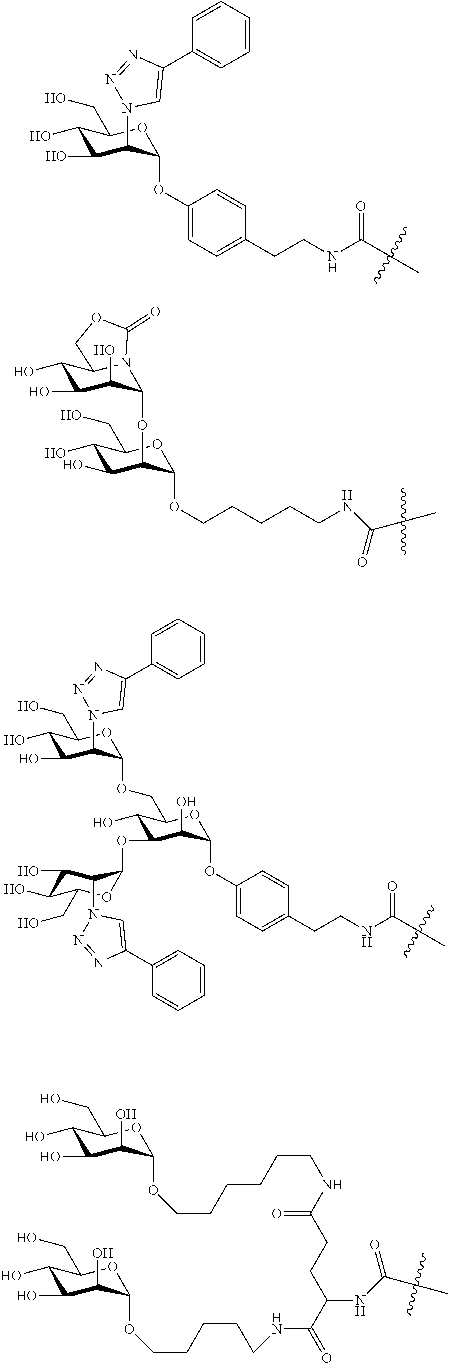

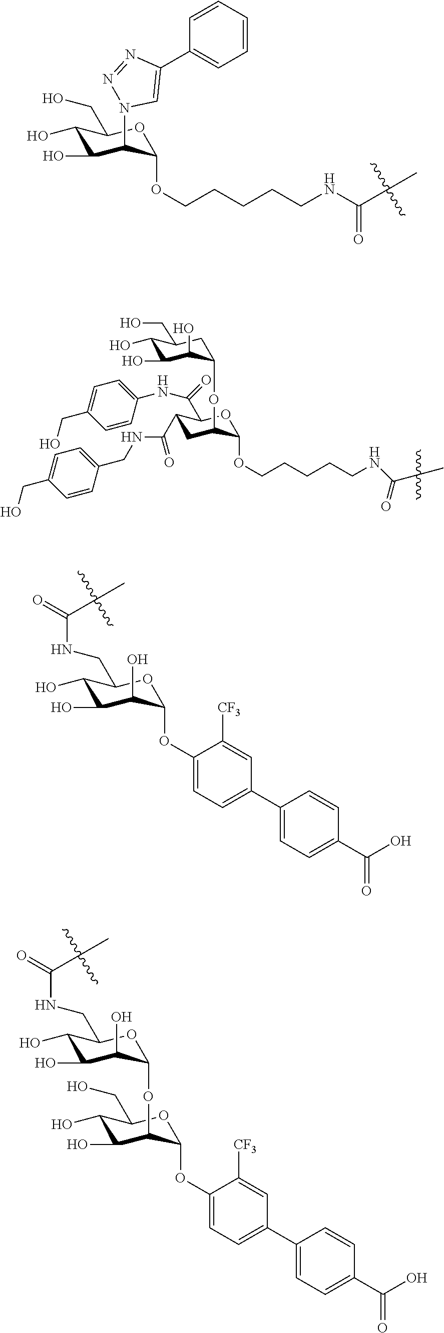

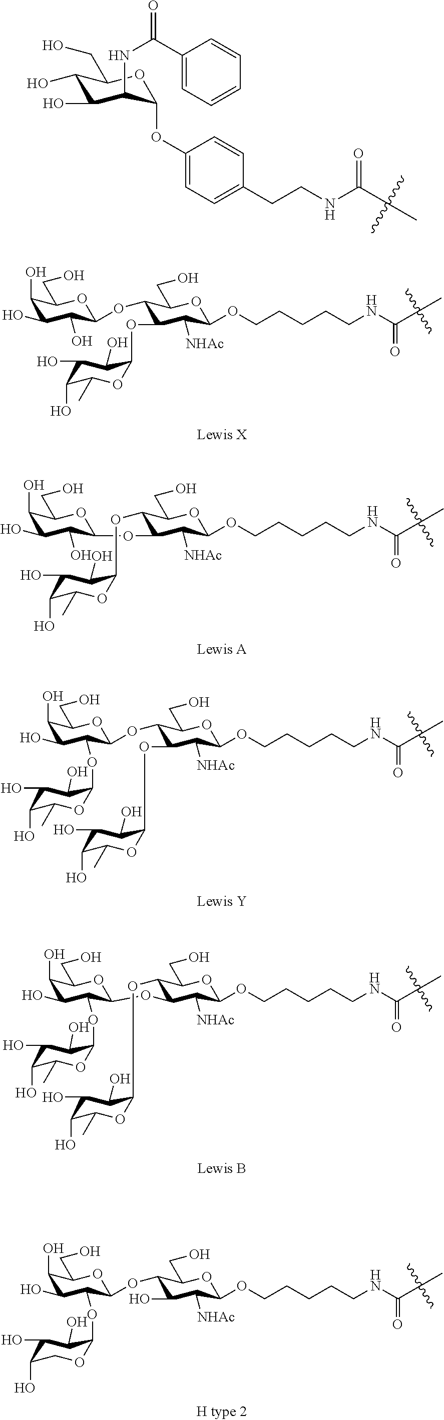

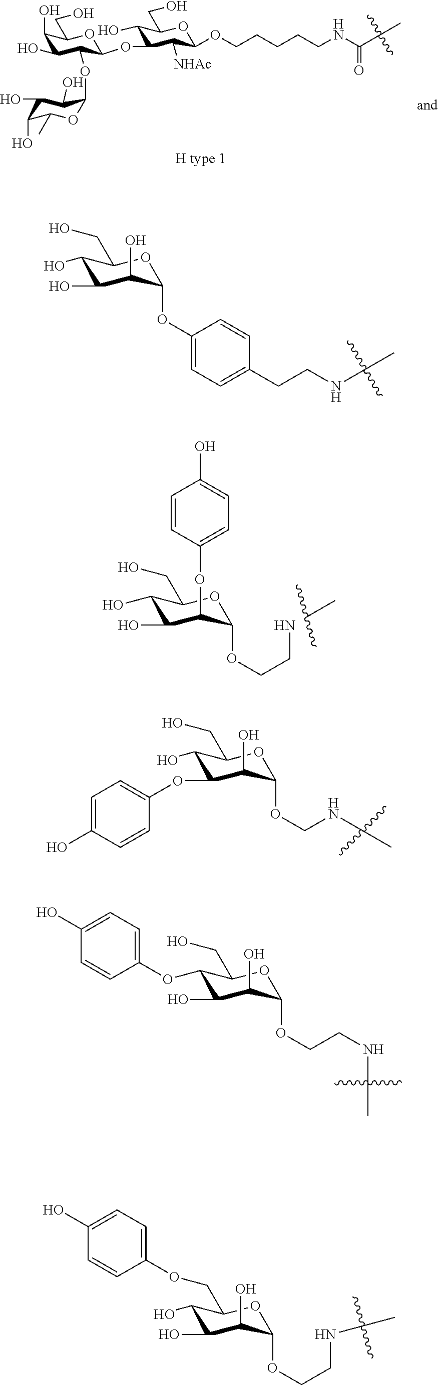

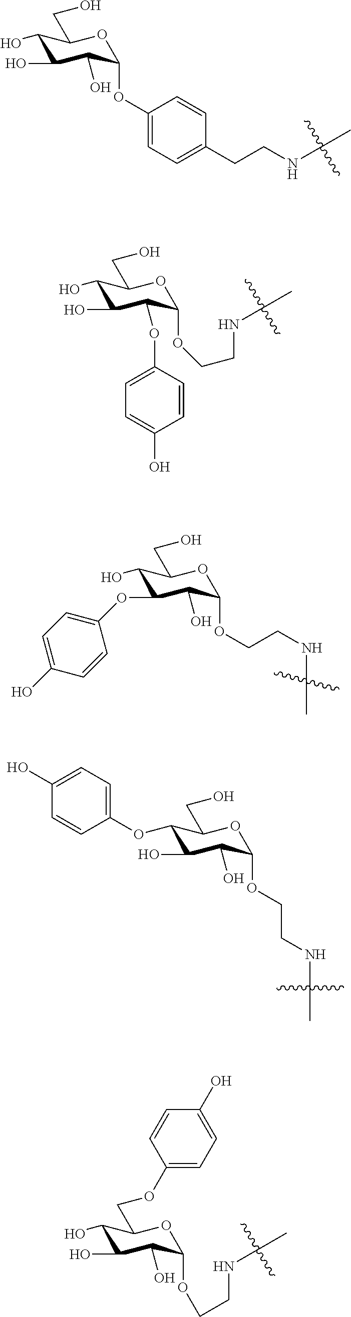

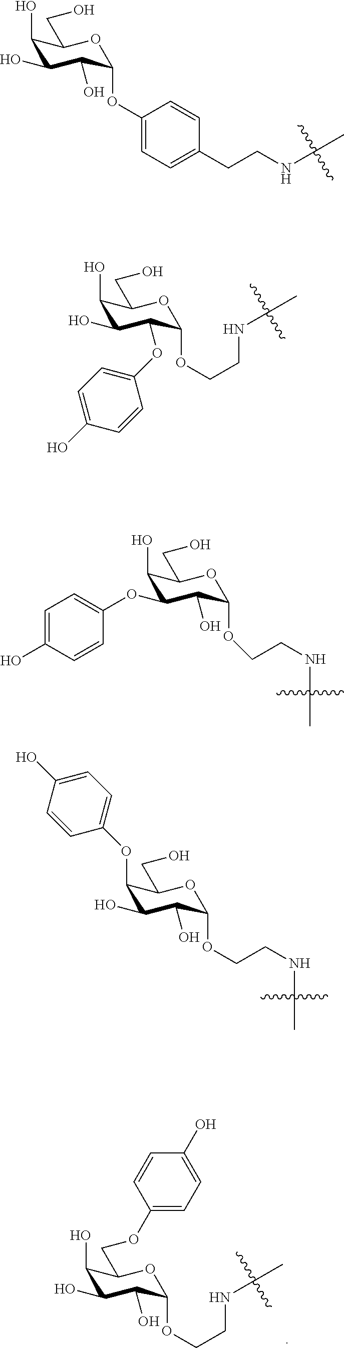

In some embodiments, the compound of the present disclosure has the structure shown in Formula 3:

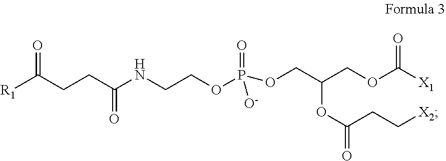

and
wherein the R₁ group is selected from the group consisting of (each structure shown below is independent from one another despite whether it is separated using a semicolon with an adjacent structure):

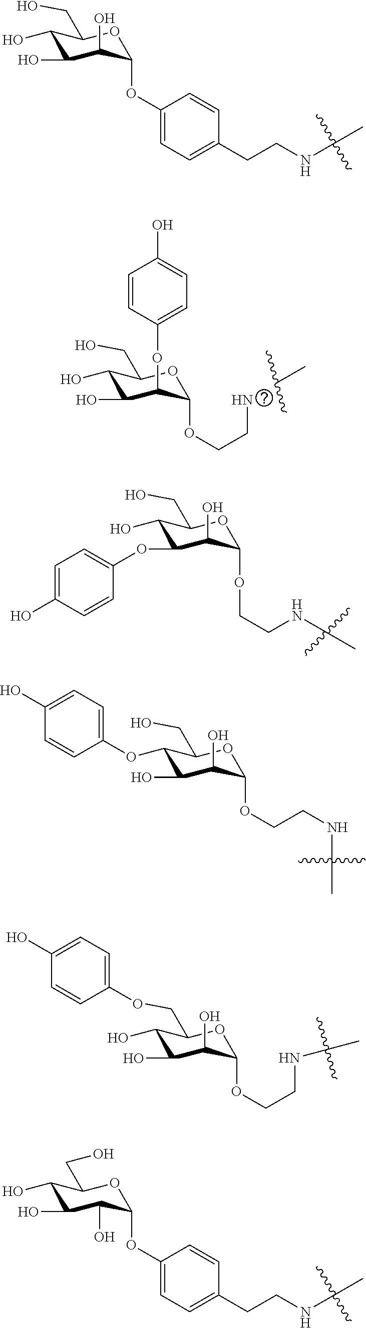

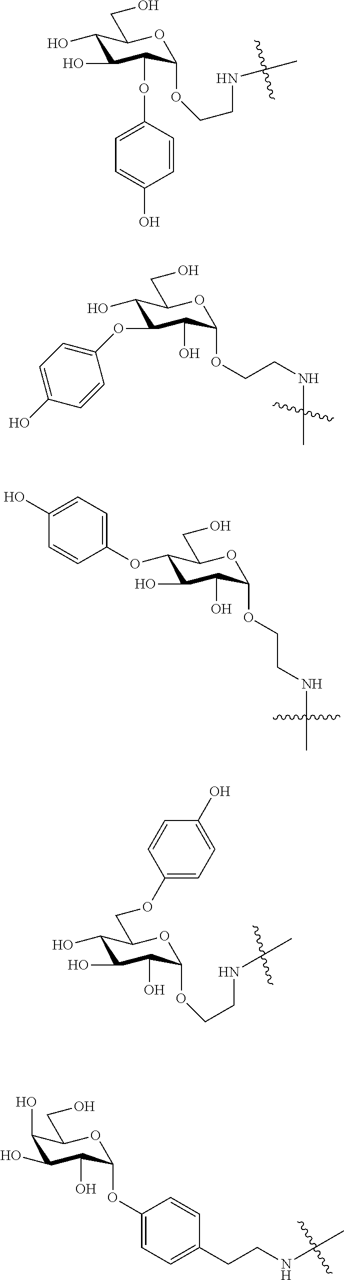

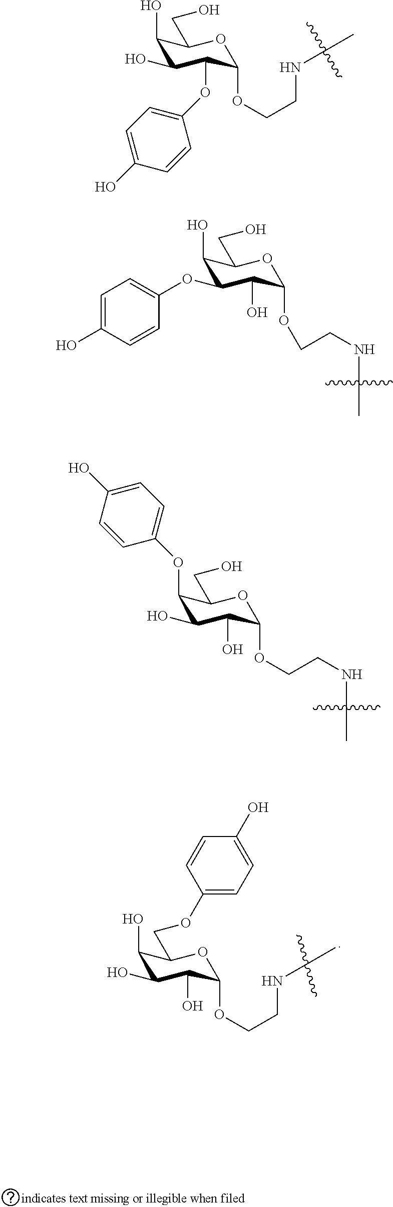

X₁ and X₂

The X₁ and X₂ are each independently hydrogen, C₁₋₃₀ alkyl, C₁₋₃₀ alkenyl, C₁₋₃₀ alkynyl, aryl, aryloxy, or a substituted version thereof, or —(CH₂)nX₄, n is 0 to 30, and X4 is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof. Without wishing to be bound by theories, at least one of the X₁ and X₂ groups is designed to provide the compound of the present disclosure with desired hydrophobicity.

In some embodiments, at least one of the X₁ and X₂ comprises a saturated hydrocarbon chain, which comprises at least 2, 3, 4, 5, 6, 7, 8, 9, 10, 11, 12, 13, 14, 15, 16, 17, 18, 19, 20, 22, 24, 26, 28, or 30 carbons, or any range of carbons defined by the foregoing endpoints, such as 2 to 30, 2 to 28, 2 to 26, 2 to 24, 2 to 20, 2 to 18, 2 to 15, 2 to 12, 2 to 10, 2 to 8, 2 to 6, 2 to 4, 3 to 30, 3 to 28, 3 to 26, 3 to 24, 3 to 20, 3 to 18, 3 to 15, 3 to 14, 3 to 13, 3 to 12, 3 to 11, 3 to 10, 3 to 9, 3 to 8, 3 to 7, 3 to 6, 3 to 5, 4 to 30, 4 to 28, 4 to 26, 4 to 24, 4 to 20, 4 to 18, 4 to 15, 4 to 14, 4 to 13, 4 to 12, 4 to 11, 4 to 10, 4 to 9, 4 to 8, 4 to 7, 4 to 6, 6 to 15, 6 to 14, 6 to 13, 6 to 12, 6 to 11, 6 to 10, 6 to 9, 6 to 8, 10 to 30, 10 to 20, 15 to 30, 15 to 28, 15 to 26, or 15 to 20 carbons.

In some embodiments, X₁ and X₂ are each independently hydrogen, C₄₋₃₀ alkyl, C₄₋₃₀ alkenyl, C₄₋₃₀ alkynyl, aryl, aryloxy, or a substituted version thereof, or —(CH₂)nX₄, n is 4 to 30, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof.

In some embodiments, X₁ and X₂ are each independently hydrogen, C₈₋₃₀ alkyl, C₈₋₃₀ alkenyl, C₈₋₃₀ alkynyl, aryl, aryloxy, or a substituted version thereof, or —(CH₂)nX₄, n is 8 to 30, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof.

In some embodiments, when one of X₁ and X₂ is hydrogen, the other one is not hydrogen. In some embodiments, when one of X₁ and X₂ is hydrogen, the other one comprises a saturated hydrocarbon chain, comprising at least 2, 3, 4, 5, 6, 7, 8, 9, 10, 11, 12, 13, 14, 15, 16, 17, 18, 19, 20, 22, 24, 26, 28, or 30 carbons, or any range of carbons defined by the foregoing endpoints, such as 2 to 30, 2 to 28, 2 to 26, 2 to 24, 2 to 20, 2 to 18, 2 to 15, 2 to 12, 2 to 10, 2 to 8, 2 to 6, 2 to 4, 3 to 30, 3 to 28, 3 to 26, 3 to 24, 3 to 20, 3 to 18, 3 to 15, 3 to 14, 3 to 13, 3 to 12, 3 to 11, 3 to 10, 3 to 9, 3 to 8, 3 to 7, 3 to 6, 3 to 5, 4 to 30, 4 to 28, 4 to 26, 4 to 24, 4 to 20, 4 to 18, 4 to 15, 4 to 14, 4 to 13, 4 to 12, 4 to 11, 4 to 10, 4 to 9, 4 to 8, 4 to 7, 4 to 6, 6 to 15, 6 to 14, 6 to 13, 6 to 12, 6 to 11, 6 to 10, 6 to 9, 6 to 8, 10 to 30, 10 to 20, 15 to 30, 15 to 28, 15 to 26, or 15 to 20 carbons. In some embodiments, one of X₁ and X₂ is C₁₅₋₃₀ alkyl, and the other is —(CH₂)nX₄, as defined above.

In some embodiments, X₄ is an aryl, aryloxy, heterocyclic group, cycloalkyl, heterocycloalkyl, or a combination thereof, and wherein X₄ comprises 0 to 6 substituents, selected from the group consisting of C₁₋₆ alkyl, halogen, C₁₋₆ alkyl halogen, and C₁₋₆ alkoxy. In certain embodiments, X₄ comprises 1 to 3 substituents. The substituent can be, but is not limited to, CH₃, CF₃, F, or OCH₃.

In some embodiments, X₄ is —R₃—O—R₄, wherein R₃ and R₄ are each independently aryl, heterocyclic group, cycloalkyl, heterocycloalkyl, each comprising 0 to 6 substituents selected from the group consisting of C₁₋₆ alkyl, halogen, C₁₋₆ alkyl halogen, and C₁₋₆ alkoxy.

In certain embodiments, X₄ is selected from the group consisting of:

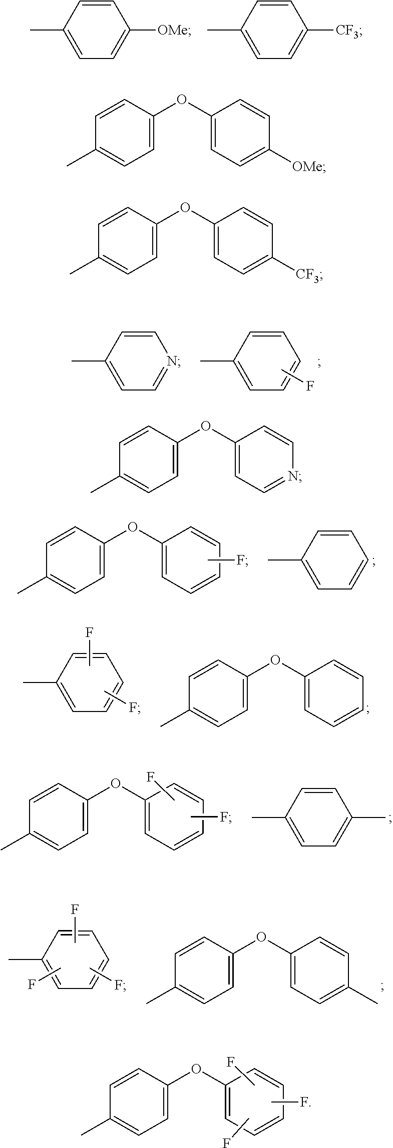

### Exemplary Compound of the Present Disclosure

This section lists some exemplary structures of the compound of the present disclosure. However, the present disclosure is not limited to the exemplary structures listed below or in the specification. In some embodiments, the compound of the present disclosure does not comprise glycolipid C34 or α-galactosylceramide (α-GalCer).

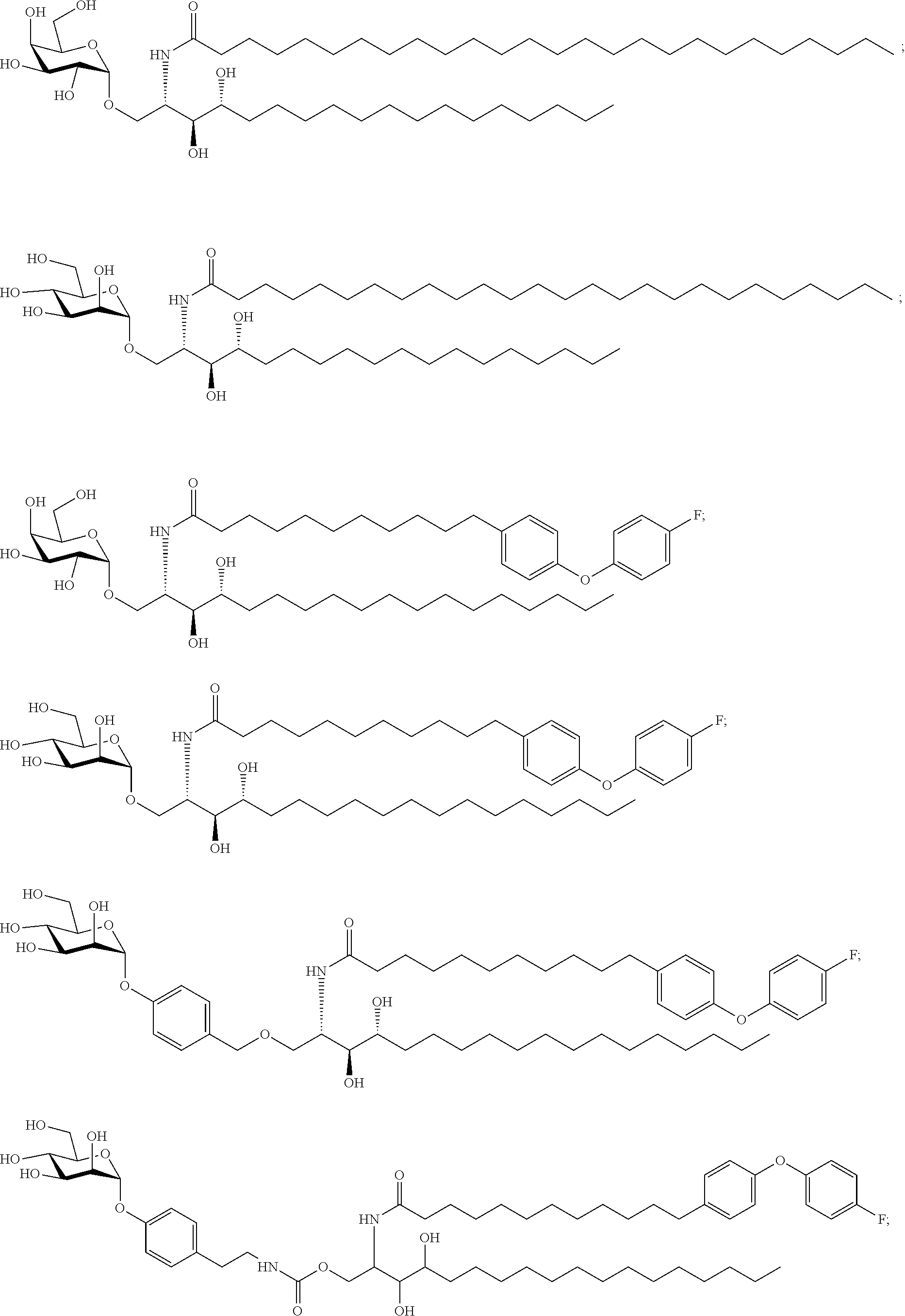

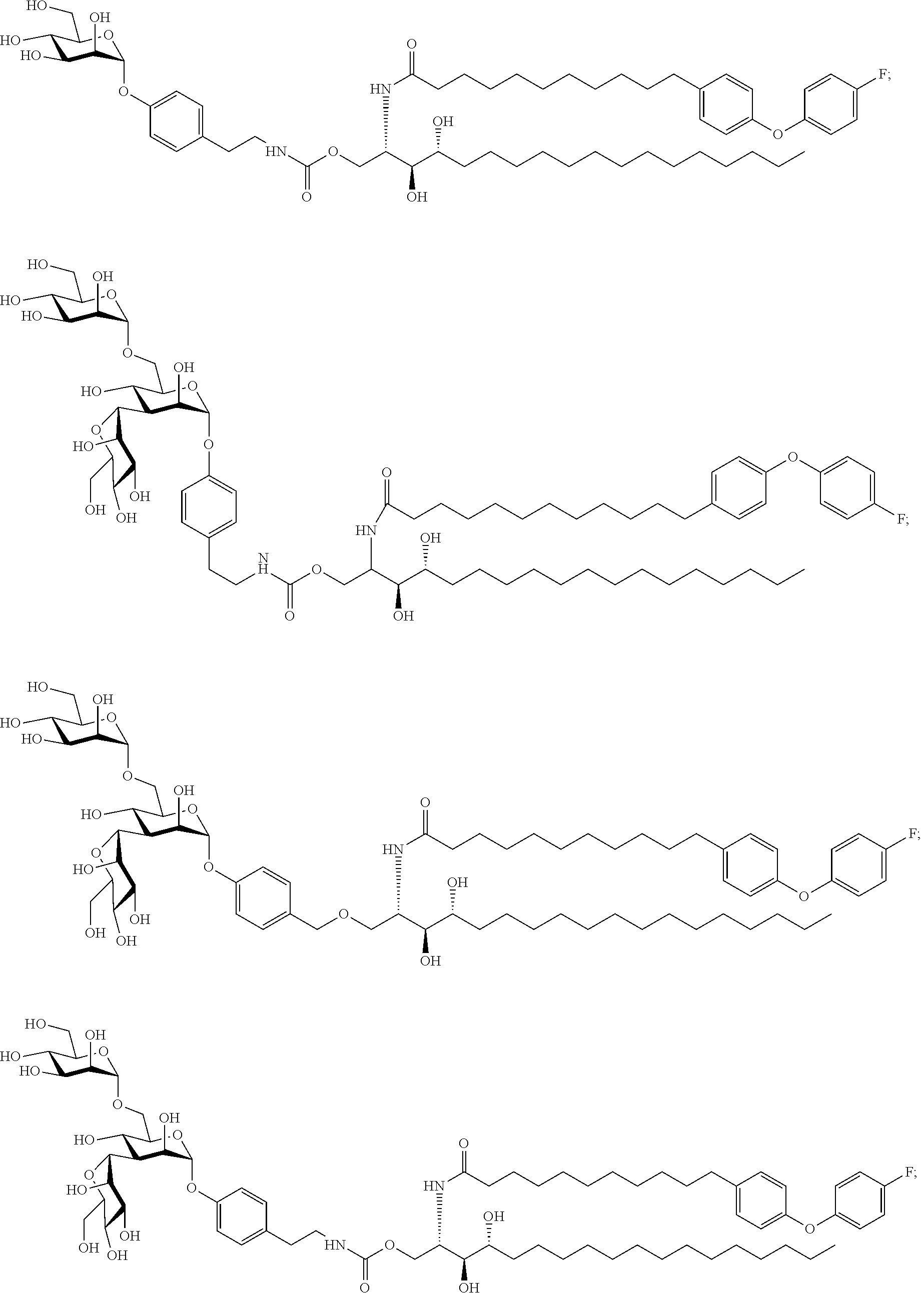

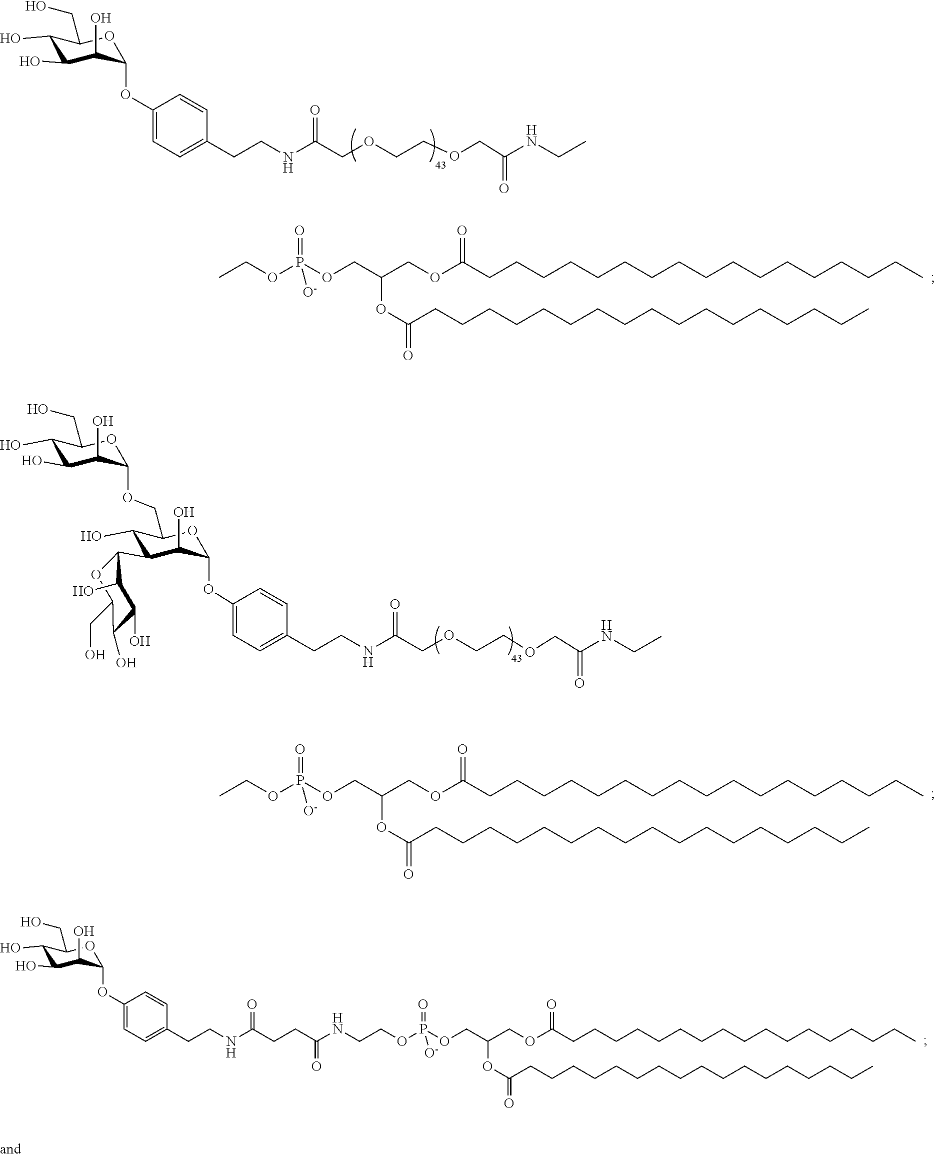

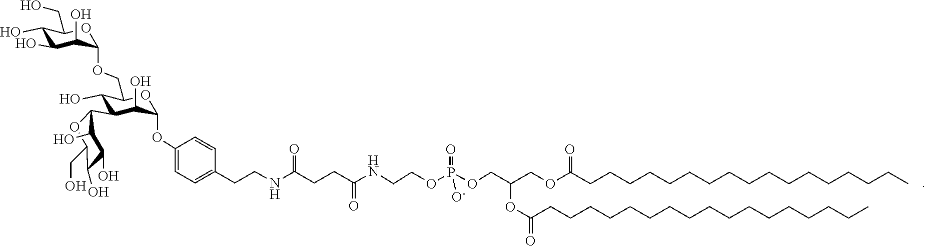

### Polymersome

Polymersomes, as disclosed herein, are enclosures, self-assembled from amphiphilic block copolymers. These amphiphilic block copolymers are macromolecules comprising at least one hydrophobic polymer block and at least one hydrophilic polymer block. When hydrated, these amphiphilic block copolymers self-assemble into enclosures such that the hydrophobic blocks tend to associate with each other to minimize direct exposure to water and form the inner surface of the enclosure, and the hydrophilic blocks face outward, forming the outer surface of the enclosure. The hydrophobic core of these aqueous soluble polymersomes may provide an environment to solubilize additional hydrophobic molecules. As such, these aqueous soluble polymersomes may act as carrier polymers for hydrophobic molecules encapsulated within the polymersomes. Moreover, the self-assembly of the amphiphilic block polymers occurs in the absence of stabilizers, which would otherwise provide colloidal stability and prevent aggregation.

### Composition and Immunogenic Composition

In one aspect, the present disclosure provides a composition comprising the nucleic acid according to an embodiment of the present disclosure. In some embodiments, the nucleic acid is encapsulated or carried by a vector as described above according to an exemplary vector of the present disclosure. The composition can be an immunogenic composition that is designed to deliver the nucleic acid according to an embodiment of the present disclosure using a vector (e.g., as described herein) to a host cell, thereby inducing an immune response against the spike protein. In some embodiments, the immune response induced has cross-activities across various kinds of coronavirus, including but not limited to a SARS-COV, MERS-COV, or SARS-COV-2 virus. In some embodiments, the immune response induced has cross-activities across various variant of a SARS-COV-2 virus, including but not limited to a SARS-COV-2 alpha variant, a SARS-COV-2 beta variant, a SARS-COV-2 delta variant, or a SARS-COV-2 omicron variant.

In some embodiments, the composition comprises at least about 0.01, 0.05, 0.1, 0.5, 1, 2, 3, 4, 5, 10, 15, 20, 25, 30, 40, 50, 60, 70, 80, 90, 95% (w/w) the vector of the present disclosure, which encapsulates or carries an exemplary nucleic acid of the present disclosure, or any range defined by the foregoing endpoints, such as, included or excluded, 0.01% to 95% (w/w), 0.01% to 90% (w/w), 0.01% to 80% (w/w), 0.01% to 70% (w/w), 0.01% to 60% (w/w), 0.01% to 50% (w/w), 0.01% to 40% (w/w), 0.01% to 30% (w/w), 0.01% to 20% (w/w), 0.01% to 10% (w/w), 0.01% to 5% (w/w), 0.01% to 1% (w/w), 0.01% to 0.1% (w/w), 0.1% to 95% (w/w), 0.1% to 90% (w/w), 0.1% to 80% (w/w), 0.1% to 70% (w/w), 0.1% to 60% (w/w), 0.1% to 50% (w/w), 0.1% to 40% (w/w), 0.1% to 30% (w/w), 0.1% to 20% (w/w), 0.1% to 10% (w/w), 0.1% to 5% (w/w), 0.1% to 1% (w/w), 1% to 95% (w/w), 1% to 90% (w/w), 1% to 80% (w/w), 1% to 70% (w/w), 1% to 60% (w/w), 1% to 50% (w/w), 1% to 40% (w/w), 1% to 30% (w/w), 1% to 20% (w/w), 1% to 10% (w/w), 1% to 5% (w/w), 5% to 95% (w/w), 5% to 90% (w/w), 5% to 80% (w/w), 5% to 70% (w/w), 5% to 60% (w/w), 5% to 50% (w/w), 5% to 40% (w/w), 5% to 30% (w/w), 5% to 20% (w/w), or 5% to 10% (w/w). The rest of the percentages of the composition can be an excipient as described herein.

In some embodiments, the composition is a pharmaceutical composition or pharmaceutical formulation. In such embodiments, the composition can further comprise a pharmaceutically acceptable excipient, adjuvant, or a combination thereof. The pharmaceutically acceptable excipient might comprise a solvent, dispersion media, diluent, dispersion, suspension aid, surface active agent, isotonic agent, thickening or emulsifying agent, preservative, polymer, peptide, protein, cell, hyaluronidase, or mixtures thereof. Various excipients for formulating pharmaceutical compositions and techniques for preparing the composition are known in the art (see Remington: The Science and Practice of Pharmacy, 22nd Edition, Edited by Allen, Loyd V., Jr, Pharmaceutical Press). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Formulation of standard pharmaceutically acceptable excipients may be carried out using routine methods in the pharmaceutical art (See Remington's Pharmaceutical Sciences, 19th Edition, Mack Publishing Company, Eastern Pennsylvania, USA.).

In certain embodiments, the adjuvant can be but is not limited to C34, Gluco-C34, 7DW8-5, C17, C23, C30, α-galactosylceramide (α-GalCer), Aluminum salt (e.g., aluminum hydroxide, aluminum phosphate, alum (potassium aluminum sulfate), mixed aluminum salts), Squalene, MF59, QS-21, Freund's complete adjuvant, Freund's incomplete adjuvant, AS03 (GlaxoSmithKline), MF59 (Seqirus), CpG 1018 (Dynavax), or a combination thereof.

### Methods of Use

In one aspect, the present disclosure provides a method for generating an immune response against coronavirus infection, comprising administering a nucleic acid of the present disclosure to a subject in need at an effective amount. In some embodiment, the immune response can be characterized by an increased immunoglobin titer (e.g., an IgG titer) in the subject (e.g., in serum collected from the subject), and the immune response can be considered as being generated if the titer is higher than a benchmark level measured before the administration. In certain embodiments, the immunoglobin titer is higher than the benchmark by about 1, 2, 3, 4, 5, 6, 7, 8, 9, or 10 logs, or any range defined by the foregoing endpoints, such as, included or excluded 1 to 10 logs, 1 to 8 logs, 1 to 6 logs, 1 to 4 logs, 2 to 9 logs, 2 to 7 logs, 2 to 5 logs, 3 to 10 logs, 3 to 8 logs, 3 to 5 logs, or 4 to 6 logs. In yet some embodiments, the immunoglobin titer is higher than the benchmark by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200%, or any range defined by the foregoing endpoints, such as, included or excluded, 5 to 200%, 5 to 150%, 5 to 100%, 5 to 75%, 5 to 50%, 5 to 25%, 10 to 200%, 10 to 175%, 10 to 125%, 10 to 100%, 10 to 75%, 10 to 50%, 10 to 25%, 25 to 200%, 25 to 150%, 25 to 100%, 25 to 75%, 25 to 50%, 50 to 200%, 50 to 175%, 50 to 125%, 50 to 100%, or 50 to 75%. In some embodiments, the measurement can be conducted using an Enzyme-linked immunosorbent assay (ELISA).

In some embodiments, generating an immune response comprises preventing the subject from being infected by the coronavirus, but the method is not so limited. As described herein, preventing the subject from being infected by the coronavirus does not necessarily mean the subject would not be infected at all but means alleviating the symptoms of coronavirus infections if the subject has been or will be infected by coronavirus.

In some embodiments, the coronavirus infection is caused by a SARS-COV, MERS-CoV, SARS-COV-2 virus, or a mixture thereof. In certain embodiments of SARS-COV-2 infection, the infection can be caused by a SARS-COV-2 alpha variant, a SARS-COV-2 beta variant, a SARS-COV-2 delta variant, a SARS-COV-2 omicron variant, or a mixture thereof.

In some embodiments, the nucleic acid is delivered by a vector. In certain embodiments, the nucleic acid is configured as an expression vector according to an embodiment of the present disclosure. In some embodiments, the nucleic acid and/or the expression vector is formulated as a composition according to an embodiment of the present disclosure.

### Administration

Regarding the methods of the present disclosure, in some embodiments, the subject is administered with a single dose of the nucleic acid of the present disclosure. Yet in some embodiments, the subject is administered with an initial dose followed by at least one booster dose, e.g., one, two, three, four, five, six, seven, eight, nine, ten, eleven, twelve, thirteen, fourteen, fifteen, sixteen, seventeen, eighteen, nineteen, twenty, or more follow-up doses, with an interval of each dose in about, 1, 2, 3, 4, 5, 6, 7 days, about 1, 2, 3, 4, 5, 6, 7, 8, 9, or 10 weeks, or about 1, 2, 3, 4, 5, 6, 7, 8, 9, 10, 11, or 12 months, or any range defined by the foregoing endpoints, such as, included or excluded, 1 to 7 days, 1 to 5 days, 1 to 3 days, 1 to 10 weeks, 1 to 8 weeks, 1 to 6 weeks, 1 to 4 weeks, 1 to 2 weeks, 1 to 12 months, 1 to 8 months, 1 to 6 months, 1 to 4 months, 1 to 2 months, or 6 to 12 months. In certain embodiments, the nucleic acid of the present disclosure encapsulating is administered twice at the same or different doses, and the two administrations are separated by 1 day, 3 days, 5 days, 1 week, 2 weeks, 1 month, 2 months, 3 months, 6 months, 1 year, 1 to 5 days, 1 to 2 weeks, 1 to 3 months, 1 to 6 months, 1 month to 1 year, 3 months to 1 year, or 6 months to 1 year.

Administration route. The nucleic acid, as described herein, may be administered (as an expression vector or a composition according to an embodiment of the present disclosure) by any route. Suitable routes include, but are not limited to, oral, nasal, mucosal, submucosal, intravenous, intramuscular, intraperitoneal, subcutaneous, intradermal, transdermal, and buccal routes. Other possible routes of administration are by spray, aerosol, or powder application through inhalation via the respiratory tract.

Effective amount of administration. The effective amount described herein refers to the amount of the nucleic acid, the expression vector comprising the nucleic acid, or the composition comprising the expression vector according to an embodiment of the present disclosure that is sufficient to provide a desired effect. In the embodiments where the purpose of administering the nucleic acid of the present disclosure is to treat or alleviate an existing infection, the effective amount refers to a therapeutically effective amount, while in some other embodiments where the purpose is to prevent infection, the effective amount refers to a prophylactically effective amount.

The effective amount of the methods of the present disclosure can be determined based on several factors, including but not limited to the conditions of the subjects (age, gender, species, body weight, health status, etc.), the progress of the disease to be treated, the administration route, the dosage and interval of the administration, and the nature of the nucleic acid (such as the stability and/or translation capacity thereof). Accordingly, the effective amount of the methods of the present disclosure is about 5, 10, 15, 20, 25, 30, 35, 40, 45, 50, 55, 60, 65, 70, 75, 80, 90, 100, 125, 150, 175, 200, 250, 300, 350, 400, 450, 500, 550, 600, 650, 700, 750, 800, 850, 900, 950 or 1000 micrograms (μg or ug), or any range defined by the foregoing endpoints, such as, include or exclude, 5 micrograms to 1000 micrograms, 5 micrograms to 900 micrograms, 5 micrograms to 800 micrograms, 5 micrograms to 700 micrograms, 5 micrograms to 600 micrograms, 5 micrograms to 500 micrograms, 5 micrograms to 400 micrograms, 5 micrograms to 300 micrograms, 5 micrograms to 200 micrograms, 5 micrograms to 175 micrograms, 5 micrograms to 150 micrograms, 5 micrograms to 125 micrograms, 5 micrograms to 100 micrograms, 5 micrograms to 90 micrograms, 5 micrograms to 80 micrograms, 5 micrograms to 70 micrograms, 5 micrograms to 60 micrograms, 5 micrograms to 50 micrograms, 5 micrograms to 40 micrograms, 5 micrograms to 30 micrograms, 5 micrograms to 20 micrograms, 5 micrograms to 10 micrograms, 10 micrograms to 1000 micrograms, 10 micrograms to 900 micrograms, 10 micrograms to 800 micrograms, 10 micrograms to 700 micrograms, 10 micrograms to 600 micrograms, 10 micrograms to 500 micrograms, 10 micrograms to 400 micrograms, 10 micrograms to 300 micrograms, 10 micrograms to 200 micrograms, 10 micrograms to 175 micrograms, 10 micrograms to 150 micrograms, 10 micrograms to 125 micrograms, 10 micrograms to 100 micrograms, 10 micrograms to 90 micrograms, 10 micrograms to 80 micrograms, 10 micrograms to 70 micrograms, 10 micrograms to 60 micrograms, 10 micrograms to 50 micrograms, 10 micrograms to 40 micrograms, 10 micrograms to 30 micrograms, 10 micrograms to 20 micrograms, 50 micrograms to 1000 micrograms, 50 micrograms to 900 micrograms, 50 micrograms to 800 micrograms, 50 micrograms to 700 micrograms, 50 micrograms to 600 micrograms, 50 micrograms to 500 micrograms, 50 micrograms to 400 micrograms, 50 micrograms to 300 micrograms, 50 micrograms to 200 micrograms, 50 micrograms to 175 micrograms, 50 micrograms to 150 micrograms, 50 micrograms to 125 micrograms, 50 micrograms to 100 micrograms, 50 micrograms to 90 micrograms, 50 micrograms to 80 micrograms, 50 micrograms to 70 micrograms, or 50 micrograms to 60 micrograms. 100 micrograms to 1000 micrograms, 100 micrograms to 900 micrograms, 100 micrograms to 800 micrograms, 100 micrograms to 700 micrograms, 100 micrograms to 600 micrograms, 100 micrograms to 500 micrograms, 100 micrograms to 400 micrograms, 100 micrograms to 300 micrograms, 100 micrograms to 200 micrograms, 100 micrograms to 175 micrograms, 100 micrograms to 150 micrograms, 300 micrograms to 1000 micrograms, 300 micrograms to 900 micrograms, 300 micrograms to 800 micrograms, 300 micrograms to 700 micrograms, 300 micrograms to 600 micrograms, 300 micrograms to 500 micrograms, 300 micrograms to 400 micrograms, 500 micrograms to 1000 micrograms, 500 micrograms to 900 micrograms, 500 micrograms to 800 micrograms, 500 micrograms to 700 micrograms, 500 micrograms to 600 micrograms, 600 micrograms to 800 micrograms, or 700 micrograms to 900 micrograms.

In some embodiments, the nucleic acid, the expression vector, or the composition comprising the expression vector is administered at a dosage level from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, per subject body weight per day, one or more times a day, to obtain the desired in vivo effect.

### Definition

Unless specifically defined otherwise, all technical and scientific terms used herein have the same meaning as commonly understood by one of ordinary skill in the art to which this disclosure belongs. Unless mentioned otherwise, the techniques employed or contemplated herein are standard methodologies well known to one of ordinary skill in the art. The practice of the present disclosure will employ, unless otherwise indicated, conventional techniques of microbiology, tissue culture, molecular biology, chemistry, biochemistry, and recombinant DNA technology, which are within the skill of the art. The materials, methods, and examples are illustrative only and not limiting. The following is presented by way of illustration and is not intended to limit the scope of the disclosure.

Numbers expressing quantities of ingredients, properties such as molecular weight, reaction conditions and results, and so forth, used to describe and claim certain embodiments of the present disclosure are to be understood as being modified in some instances by the term “about.” A skilled artisan in the field would understand the meaning of the term “about” in the context of the value that it qualifies. The numerical values presented in some embodiments of the present disclosure may contain certain errors resulting from the standard deviation in their respective testing measurements. For example, the term “about,” as used herein, refers to a measurable value such as an amount, a temporal duration, and the like and is meant to encompass variations of ±20%, ±10%, ±5%, ±1%, or ±0.1% from the specified value, as such variations are appropriate.

As used herein, “substantially” means sufficient to work for the intended purpose. The term “substantially” thus allows for minor, insignificant variations from an absolute or perfect state, dimension, measurement, result, or the like, such as expected by a person of ordinary skill in the field, but that does not appreciably affect overall performance. When used with respect to numerical values or parameters or characteristics expressed as numerical values, “substantially” means within ten percent.

As used herein, “treat,” “treatment,” and “treating” refer to an approach for obtaining beneficial or desired results, for example, clinical results. For the purposes of this disclosure, beneficial or desired results may include inhibiting or suppressing the initiation or progression of an infection or a disease, ameliorating or reducing the development of symptoms of an infection or disease, or a combination thereof.

As used herein, “preventing” and “prevention” are used interchangeably with “prophylaxis” and can mean complete prevention of infection or prevention of the development of symptoms of that infection, a delay in the onset of a disease or its symptoms, or a decrease in the severity of a subsequently developed infection or its symptoms.

As used herein, “recombinant” modifying a protein describes that the protein is designed to be produced by introducing an engineered nucleic acid into a host organism, like bacteria, yeast, or mammalian cells, using laboratory or industrial processes.

As described herein, percent identity between two polypeptides or nucleic acid sequences is determined in various ways that are within the skill in the art, for instance, using publicly available computer software such as Smith Waterman Alignment (Smith and Waterman (1981) J Mol Biol 147:195-7); “BestFit” (Smith and Waterman, Advances in Applied Mathematics, (1981) 482-489) as incorporated into GeneMatcher Plus™, Schwarz and Dayhof, Atlas of Protein Sequence and Structure, Dayhof, M. O., Ed (1979) 353-358; BLAST program (Basic Local Alignment Search Tool; (Altschul et al. (1990) J Mol Biol 215:403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the full length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

As used herein, “glycan” or “glycosyl group refers to a polysaccharide, oligosaccharide, or monosaccharide. Glycans can be monomers or polymers of sugar residues and can be linear or branched. A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′ sulfo N-acetylglucosamine, etc.).

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In some aspects, the adults are seniors about 65 years or older, or about 60 years or older. In some aspects, the subject is a pregnant woman or a woman intending to become pregnant. In other aspects, subject is not a human; for example, a non-human primate; for example, a baboon, a chimpanzee, a gorilla, or a macaque. In certain aspects, the subject may be a pet, such as a dog or cat.

As used herein, “alkyl” refers to a hydrocarbon chain that may be a straight chain or branched chain, saturated or unsaturated, containing the indicated number of carbon atoms. For example, C1-6 indicates that the group may have from 1 to 6 (inclusive) carbon atoms in it. Non-limiting examples include methyl, ethyl, iso-propyl, tert-butyl, n-hexyl. A “heteroalkyl” group is an alkyl group in which at least one carbon of the chain has been replaced by a heteroatom. In some embodiments, the heteroalkyl group has 1 to 20 carbon atoms. The term “alkoxy” is intended to mean the moiety —OR, where R is alkyl. The term “aryloxy” is intended to mean the moiety —OR, where R is aryl.

As used herein, “alkenyl” refers to a hydrocarbon chain including at least one double bond, which may be a straight chain or branched chain, and containing the indicated number of carbon atoms. For example, C₂₋₆ indicates that the group may have from 2 to 6 (inclusive) carbon atoms in it. Non-limiting examples include ethenyl and prop-1-en-2-yl.

As used herein, “alkynyl” refers to a hydrocarbon chain including at least one triple bond, which may be a straight chain or branched chain, and containing the indicated number of carbon atoms. For example, C₂₋₆ indicates that the group may have from 2 to 6 (inclusive) carbon atoms in it. Non-limiting examples include ethynyl and 3,3-dimethylbut-1-yn-1-yl.

As used herein, “cycloalkyl” refers to a nonaromatic cyclic, bicyclic, fused, or spiro hydrocarbon radical having 3 to 10 carbons, such as 3 to 8 carbons, such as 3 to 7 carbons, wherein the cycloalkyl group, which may be optionally substituted. Examples of cycloalkyls include five-membered, six-membered, and seven-membered rings. A cycloalkyl can include one or more elements of unsaturation; a cycloalkyl that includes an element of unsaturation is herein also referred to as a “cycloalkenyl”. Examples include cyclopropyl, cyclobutyl, cyclopentyl, cyclopentenyl, cyclohexyl, cyclohexenyl, cycloheptyl, and cyclooctyl.

As used herein, “heterocycloalkyl” refers to a nonaromatic 5-8 membered monocyclic, 8-12 membered bicyclic, or 11-14 membered tricyclic ring fused or spiro system radical having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Heterocycloalkyls can also include oxidized ring members, such as —N(O)—, —S(O)—, and —S(O)₂—. Examples of heterocycloalkyls include five-membered, six-membered, and seven-membered heterocyclic rings. Examples include piperazinyl, pyrrolidinyl, dioxanyl, morpholinyl, tetrahydrofuranyl, and the like.

As used herein, “aryl” or “aryl group” refers to a moiety formed by the removal of one or more hydrogen (“H”) or deuterium (“D”) from an aromatic compound. The aryl group may be a single ring (monocyclic) or have multiple rings (bicyclic, or more) fused together or linked covalently. A “carbocyclic aryl” has only carbon atoms in the aromatic ring(s). A “heteroaryl” is intended to mean an aromatic ring system containing 5 to 14 aromatic ring atoms that may be a single ring, two fused rings or three fused rings wherein at least one aromatic ring atom is a heteroatom selected from, but not limited to, the group consisting of O, S and N. Heteroaryls can also include oxidized ring members, such as —N(O)—, —S(O)—, and —S(O)₂—. Examples include furanyl, thienyl, pyrrolyl, imidazolyl, oxazolyl, thiazolyl, isoxazolyl, pyrazolyl, isothiazolyl, oxadiazolyl, triazolyl, thiadiazolyl, pyridinyl, pyrazinyl, pyrimidinyl, pyridazinyl, triazinyl and the like. Examples also include carbazolyl, quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, carbazolyl, acridinyl. phenazinyl, phenothiazinyl, phenoxazinyl, benzoxazolyl, benzothiazolyl, 1H-benzimidazolyl, imidazopyridinyl, benzothienyl, benzofuranyl, isobenzofuran and the like.

As used herein, “amine” refers to a compound that contains a basic nitrogen atom with a lone pair. The term “amino” refers to the functional group or moiety —NH₂, —NHR, or —NR₂, where R is the same or different at each occurrence and can be an alkyl group or an aryl group.

As used herein, “halogen” or “halo” refers to fluorine, bromine, chlorine, or iodine. In particular, it typically refers to fluorine or chlorine when attached to an alkyl group and further includes bromine or iodine when on an aryl or heteroaryl group.

As used herein, the term “haloalkyl” refers to an alkyl as defined herein, which is substituted by one or more halo groups. The haloalkyl can be monohaloalkyl, dihaloalkyl, trihaloalkyl, or polyhaloalkyl, including perhaloalkyl. A monohaloalkyl can have one chloro or fluoro within the alkyl group. Chloro and fluoro are commonly present as substituents on alkyl or cycloalkyl groups; fluoro, chloro, and bromo are often present on aryl or heteroaryl groups. Dihaloalkyl and polyhaloalkyl groups can have two or more of the same halo atoms or a combination of different halo groups on the alkyl. Typically, the polyhaloalkyl contains up to 12, or 10, or 8, or 6, or 4, or 3, or 2 halo groups. Non-limiting examples of haloalkyl include fluoromethyl, difluoromethyl, trifluoromethyl, chloromethyl, dichloromethyl, trichloromethyl, 2,2,2-trifluoroethyl, pentafluoroethyl, heptafluoropropyl, difluorochloromethyl, dichlorofluoromethyl, difluoroethyl, difluoropropyl, dichloroethyl and dichloropropyl. A perhalo-alkyl refers to an alkyl having all hydrogen atoms replaced with halo atoms, e.g., trifluoromethyl.

As used herein, unless otherwise specified, the term “heteroatom” refers to a nitrogen (N), oxygen (O), or sulfur(S) atom.

### EXAMPLE

### Example A1: Identifying Conserved Sequences Among Prevalent Variants for Designing Low-Sugar Vaccines

Conserved Epitope Identification. A total of 14,624,495 SARS-COV-2 S protein sequences and their variant information were extracted from the GISAID database (version: Mar. 3, 2023) for this study, including Alpha (B.1.1.7), Beta (B.1.351), Gamma (P.1), Delta (B.1.617.2), Omicron (BA.1, BA.2, BA.3, BA.4, BA.5, BA.2.12.1, BA.2.75.*). The top-ranked emerging variants (spread) reported by GISAID are BA.2.47, BQ.1, BQ.1.1, BQ.1.1.28, BQ.1.1.32, CH.1.1.3, EG.5.1, EL.1, EU.1.1, FD.1.1, XBB.1.16, XBB.1.16.6, XBB.1.17.1, XBB.1.5, XBB.1.5.10, XBB.1.5.59, XBB.1.9.1, XBB.1.9.2, XBB.2.3, XBB.2.3.3, XBB.2.3.8, and XBF. (version: Feb. 8, 2023, Apr. 24, 2023, Jun. 13, 2023, and Aug. 19, 2023). The S protein sequences and their variant information were used for amino acid mutation rate calculation. The linear conserved epitopes in this study are 10-20 contiguous amino acids in length and the state of the residues are mostly exposed or exposed but shielded by glycans. All amino acid mutation rates in the conserved epitope sequences should be <1% and not affected by mutations in the dominant virus strains and the top-ranked emerging variants. All variants are double-confirmed by GISAID and CoV-SPECTRUM. All conserved sequences are confirmed by IEDB as epitopes with 100% concordance or as their subsequences.

The 3D structural models of the SARS-COV-2 Spike protein (S protein) with representative glycan profiles were obtained as described in H.-Y. Huang, et al., Vaccination with SARS-COV-2 spike protein lacking glycan shields elicits enhanced protective responses in animal models. Sci. Transl. Med. 14, 21 (2022), which is hereby incorporated by reference in its entirety. Briefly, the S protein 3D structure modeling was constructed by using CHARMM-GUI and OpenMM based on the Protein Data Bank (PDB), with the most abundant glycoform of BEAS-2B data as representative glycan profile. Scripts, parameters, and preoptimized models generated by CHARMM-GUI were used as the input for OpenMM. The protein secondary structure was determined by majority voting in the Dictionary of Secondary Structure of Proteins (DSSP) program and 2Struc web server. The probe radius of 7.2 Å, mimicking the hypervariable loops in the complementarity determining region of antibodies, was used in the FreeSASA program to calculate each RSA of residue in S protein, both with and without representative glycans. Residues with RSA above 5% were regarded as exposed, otherwise as buried. Glycans are considered to provide shielding for the residues with buried states in models with glycans whereas these same residues have exposed states in models without glycans.

The above analysis identifies 17 conserved epitopes from 14 million S protein sequences. One of these epitopes (E1; SEQ ID NO: 15) is in the NTD, three (E2 to E4; SEQ ID NO: 16, SEQ ID NO: 17, and SEQ ID NO: 18) in RBD, two (E5 and E6; SEQ ID NO: 19 and SEQ ID NO 20) in SD1/2, and 11 (E7 to E17; SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, and SEQ ID NO: 31) in the S2 domain, including six in the CD/HR2 (the stem) region, which are the most conserved epitopes in this analysis. Except for E17, all conserved epitopes were shielded by glycans, suggesting that during the antigen presentation process, the glycans may not be completely processed and thus may shield the conserved epitopes from immune response. In the figure, single asterisks indicate the eight conserved epitopes with less than 0.5% mutation rate of each amino acid and double asterisks indicate the nine highly conserved epitopes with less than 0.1% mutation rate of each residue. Interestingly, six of these highly conserved epitopes are concentrated in the stem (E12 to E17) and three in the RBD (E2 to E4). It is also noted that E3 is in the receptor-binding motif (RBM, 438 to 506) of RBD, the site for S protein binding to ACE2. The conservation of E3 suggests that it is probably essential for pathogen-host interaction.

All residues of S protein were categorized into three categories for analysis: buried, shielded, and exposed residues (the residue number in each category is 851:172:250). The average mutation rates of the three categories were 0.84%, 1.67%, and 3.66%, and the standard deviations were 5.90%, 8.33%, and 11.58%, respectively. Buried residues are those not easily recognized by the immune system and have the lowest mutation rate, followed by shielded residues and the highest exposed residues. Based on this analysis, the conserved S protein epitopes are concentrated in the stem region of S2. Thus, deleting the 9 glycosites in S2 or the 6 glycosites in the stem to generate the low-sugar spike protein or its mRNA as vaccine is expected to better expose the conserved and glycan-shielded epitopes to the immune system and thus elicit broadly protecting immune responses against the conserved epitopes.

SARS-COV-2 and SARS-COV share the same epitopes in the stem. Based on a pairwise sequence alignment between the S protein sequences of SARS-COV-2 and SARS-COV, 5 of the 17 conserved SARS-COV-2 epitopes are identical to SARS-COV (E13-E17), and interestingly, they are all located in the stem region (CD and HR2). Among the 6 conserved epitopes (E12-E17) in the stem of the SARS-COV-2 S protein, 5 epitopes (E12-E16) are shielded by glycans. Moreover, of these 6 epitopes (E12-E17), E14 has residue mutation rates of less than 0.5%, while the other four epitopes have residue mutation rates of less than 0.1%. This indicates that the conservation of the stem is relatively high, making it a suitable target for development of broadly protective vaccines

### Example A2: Preparation of Exemplary Low-Sugar Vaccines

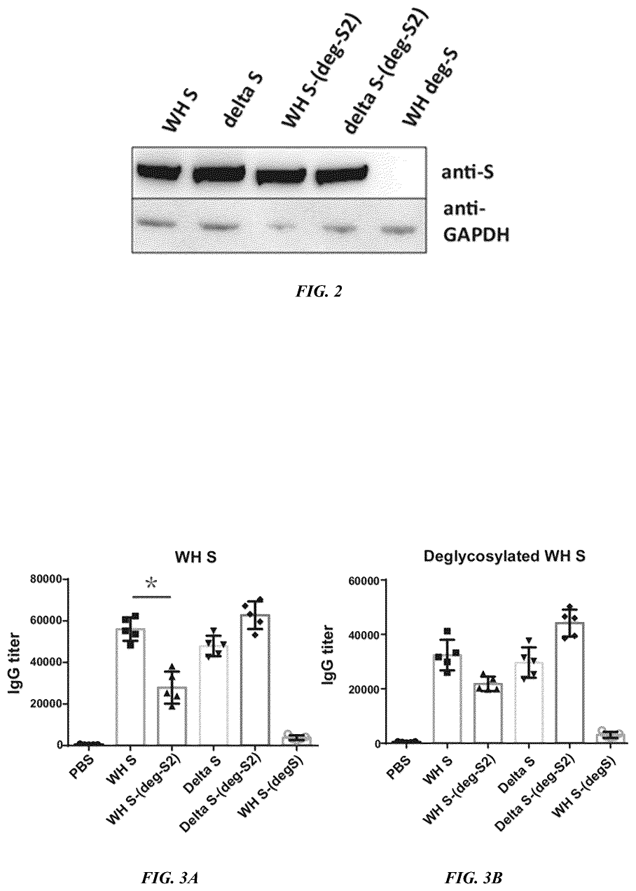

mRNA Vaccine of Deglycosylated S Protein and Formulation. The prefusion state of the S, the codon-optimized S gene of SARS-COV-2 was synthesized by GenScript and cloned into pVax or pMRNA™, and was stabilized by proline substitutions at positions K968 and V969 (S-2P). To delete the N-glycosites, the putative sequon N-X-S/T was changed to Q-X-S/T by using site-directed mutagenesis on the S-2P expression plasmid. For the in-vitro transcription, the linear DNA with the T7 promoter, 5′ untranslated region, 3′ untranslated region, S-2P, and poly(A) tail signal sequence was amplified by using TOOLS Ultra High Fidelity DNA Polymerase (BIOTOOLS Co., Ltd.) with 1 μL of the DNA template in an mMESSAGE mMACHINE® Kit (Thermo Scientific) at 37° C. for 1 hour according to the manufacturer's protocol. The mRNA was purified by an RNA cleanup kit (BioLabs), according to the manufacturer's protocol, and stored at −80° C. until further use. It was noticed that the mRNA of Wuhan (WH) strain S protein with deletion of all-glycosites (deg-S) did not express a S protein that can be recognized by the anti-S protein antibody (FIG. 2), suggesting glycosylation is critical to protein folding, and removal of glycosylation sites requires evaluation and verification.

The mRNA that encoded the S protein, the S protein with deletion of S2 glycosites, or the S protein with deletion of all glycosites was then encapsulated in lipid nanoparticles (LNPs) to form mRNA-LNP for immunization in mice. For the formulation mRNA-LNP, the mRNA that encoded the S protein, the S protein with deletion of S2 glycosites, or the S protein with deletion of all glycosites was then encapsulated in lipid nanoparticles (LNPs) to form mRNA-LNP for immunization in mice using a self-assembly process in which an aqueous solution of mRNA at pH 4.0 was rapidly mixed with an ethanolic lipid mixture containing ionizable cationic lipid, phosphatidylcholine, cholesterol, and polyethylene glycol-lipid. The compositions of LNP were DSPC (Sigma), cholesterol (Sigma), DOTAP (Sigma), and DMG-PEG 2000 (Sigma). The mRNA-LNP was characterized and subsequently stored at −80° C. at a concentration of 1 mg/mL. After HEK293 cells were transfected with 10 μg of mRNA-LNP in six wells of a plate at 48 h, the total cell lysate was collected to monitor the expression of S by Western blot.

### Example A3: Immune Responses Induced by the Exemplary Low-Sugar Vaccines

Animals and immunizations. BALB/c mice aged 6-8 weeks old (n=5) were immunized intramuscularly with 50 μg mRNA-LNP in PBS with 300 mM sucrose. Animals were immunized at week 0, boosted with a second vaccination at week 2, and serum samples and spleens were collected from each mouse one week after the booster immunization.

Serum IgG titer measure. Anti-S protein ELISA was used to determine IgG titer. Plates were coated with 50 ng/well of variant S protein and then blocked with 5% skim milk. The serum from immunized mice and HRP-conjugated secondary antibodies were sequentially added. Peroxidase substrate solution (TMB) and 1M H₂SO₄ stop solution were used and absorbance (OD 450 nm) was read by a microplate reader.

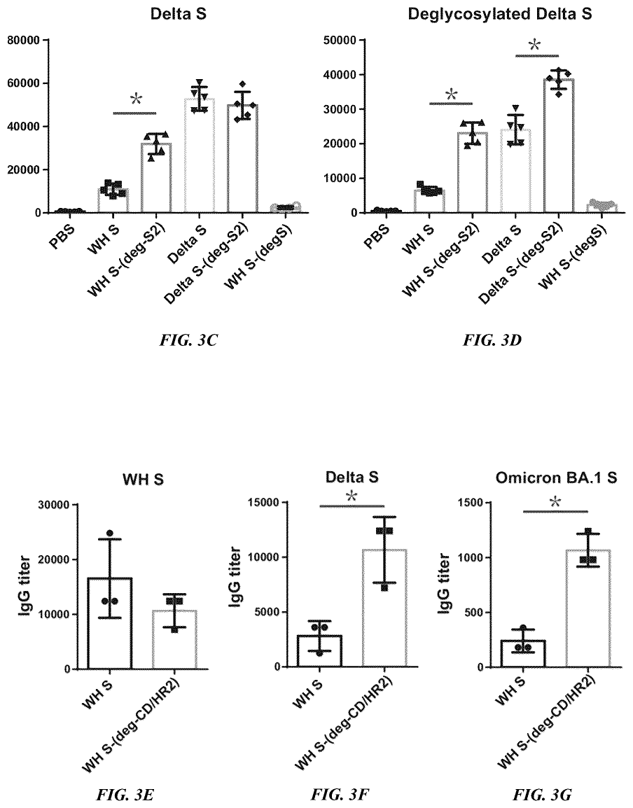

Compared to the unmodified spike mRNA vaccine, sera from mice immunized by the spike mRNA with deletion of all S2 N-glycosites in WH S [WH S-(deg-S2)] elicited a slightly lower IgG titer against the fully glycosylated WH S (FIG. 3A) or deglycosylated WH S protein (FIG. 3B) but a higher (˜sevenfold) IgG titer against the fully glycosylated Delta S (FIG. 3C) or deglycosylated Delta S protein in enzyme-linked immunosorbent assay (ELISA) (FIG. 3D). Similarly, the Delta spike mRNA vaccine with deletion of glycosites in S2 [Delta S-(deg-S2)] elicited a slightly lower IgG titer against the fully glycosylated Delta S protein (FIG. 3C) but a higher IgG titer against the fully glycosylated or deglycosylated WH S (FIG. 3A and FIG. 3B) and the deglycosylated Delta S protein (FIG. 3D). A similar result was observed in the case of spike mRNA vaccine with deletion of the six glycosites in the stem region [WH S-(deg-CD/HR2)] (FIG. 3E, FIG. 3F, and FIG. 3G). The mice immunized with WH S-(deg-CD/HR2) mRNA vaccine induced a stronger antibody response (˜10-fold) with a higher IgG titer against the S protein of Delta (FIG. 3F) and Omicron BA.1 (FIG. 3G) variants as compared to the unmodified mRNA, suggesting that deleting the glycan shield in the stem would enhance antibody response to the stem with cross-reactivity against the S protein of other variants containing the conserved epitopes.

Pseudovirus neutralization assay for serum study. To analyze the effect of S2 glycosite deletion on the neutralization activity of antibodies generated from immunized mice, the pseudovirus neutralization assay was performed. SARS-COV-2 pseudovirus variants were constructed by the RNAi Core Facility at Academia Sinica using the procedure described previously (8). The pseudotyped lentivirus was then stored at −80° C. To estimate the lentiviral titer by AlarmaBlue assay (Thermo Scientific), the transduction unit (TU) of pseudotyped lentivirus was estimated by using a cell viability assay. HEK-293T cells expressing the human ACE2 gene were plated on a 96-well plate one day before lentivirus transduction. To determine the titer of pseudotyped lentivirus, different amounts of lentivirus were added into the culture medium containing polybrene (final concentration 8 μg/ml) (Sigma), and spin infection was carried out at 1,100×g in 96-well plate for 30 min at 37° C. After incubation for 16 h, the culture medium containing virus and polybrene was removed and replaced with fresh complete DMEM containing 2.5 μg/ml puromycin (Sigma). After treating puromycin for 48 h, the culture medium was removed, and the cell viability was detected by using AlarmaBlue reagents according to the manufacturer's instructions. The survival rate of uninfected cells was set as 100%, and the virus titer was determined by plotting the survival cells versus the diluted viral dose. The SARS-COV and MERS-COV pseudoviruses were purchased from eEnzyme.

For neutralization assay, heat-inactivated sera or antibodies were serially diluted with desired dilution and incubated with 1,000 TU of SARS-COV-2 pseudotyped lentivirus in DMEM for 1 h at 37° C. The mixture was then inoculated with 10,000 HEK-293T cells stably expressing the human ACE2 gene or Huh7 (for MERS-COV pseudovirus) in a 96-well plate. The culture medium was replaced with fresh complete DMEM (supplemented with 10% FBS and 100 U/ml Penicillin/Streptomycin) at 16 h post-infection and continuously cultured for another 48 h. The expression level of the luciferase gene was determined by using Bright-Glo™ Luciferase Assay System (Promega). The relative light unit (RLU) was detected by Tecan i-control (Infinite 500). The percentage of inhibition was calculated as the ratio of RLU reduction in the presence of diluted serum to the RLU value of no serum control and the calculation formula as shown below: (RLU^{control}−RLU^{Serum})/RLU^{control}.

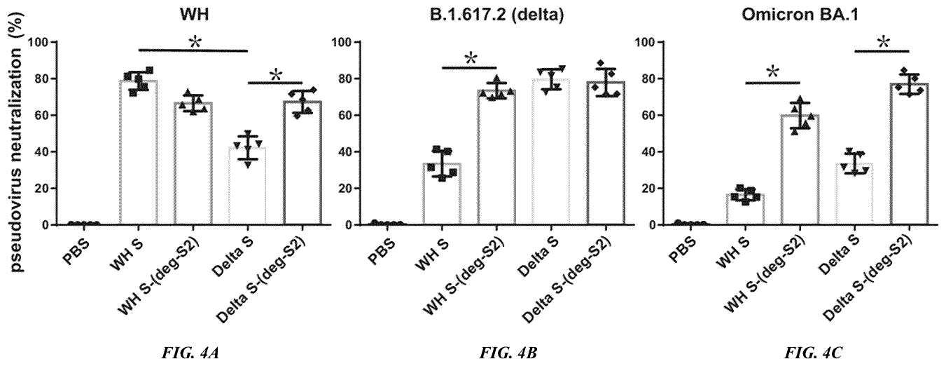

The result showed that the WH and Delta mRNA vaccines with deletion of glycosites in S2 generated antibodies with slightly reduced (by ˜10%) neutralization activity against WH or Delta pseudovirus, respectively (FIG. 4A and FIG. 4B), but with increased (fivefold to eightfold) neutralization activity against the Omicron variants including BA.1 (FIG. 4C), BA.1.12.1 (FIG. 4D), BA.2 (FIG. 4E), BA.4/5 (FIG. 4F), than the unmodified mRNA. Without wishing to be bound by theories, this finding of a broader protection suggests enhanced antibody and T cell responses after deletion of the shielded glycans.

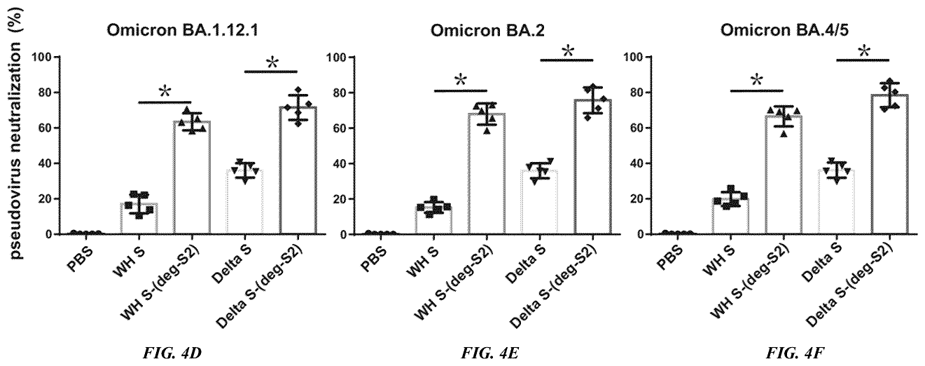

Measurement of GrzB-secreting cells. To characterize the T cell response, splenocytes from immunized mice were isolated and incubated with the peptide pool of WH S, RBD, and S2 protein to measure granzyme B (GrzB)-secreting T cells by enzyme-linked immune absorbent spot (ELISpot) analysis. A total of 5×10⁵ splenocytes from immunized mice were ex vivo restimulated with full-length WH S, RBD, and S2 peptide mix (0.1 μg/ml final concentration per peptide) (Sino Biologicals) in the GrzB ELISpot assays (R&D Systems) according to the manufacturer's instructions and spots were counted. It was found that the mRNA vaccine with deletion of S2 glycosites induced more GrzB-secreting cells than the unmodified spike mRNA of WH and Delta (FIG. 5A, FIG. 5B, and FIG. 5C).

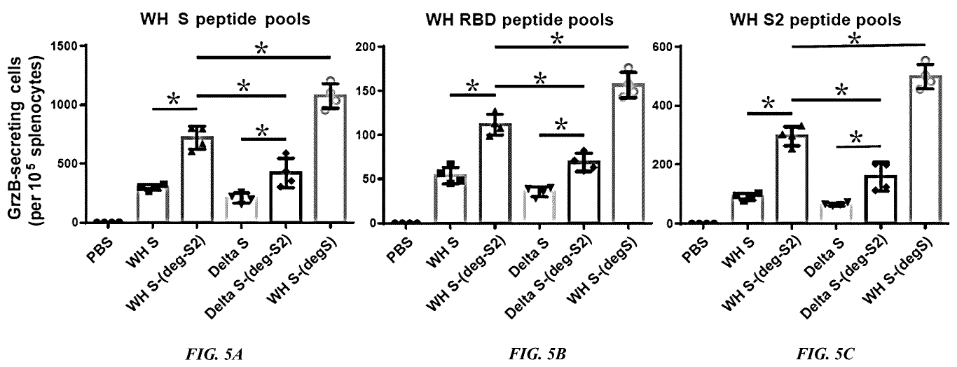

DNA plasmid transfection and MG132 treatment. Furthermore, T cell response induced by the exemplary low-sugar vaccines were tested. After the HEK293 cell was seeded in the 6 well plates, cells were transfected with 3 μg of each plasmid by TransIT®-LT1 Transfection Reagent (Mirus) and then incubated with 1 μM MG-132 (MedChemExpress) or DMSO at 37° C. for 24 h. The total lysate was collected and the variant S expression was analyzed by western blot. The results indicate that the Delta S-(deg-S2) with more stable S protein expression than that of the WH S-(deg-S2) showed a weaker T cell response (FIG. 6). Interestingly, the WH S-(deg-S) vaccine with all glycosites deleted showed the lowest stable protein expression but the highest T cell response. These results indicate that unlike the case in vitro, the stability and integrity of the S protein generated in vivo affects T cell response and the misfolded S protein seemed to induce a stronger T cell response.

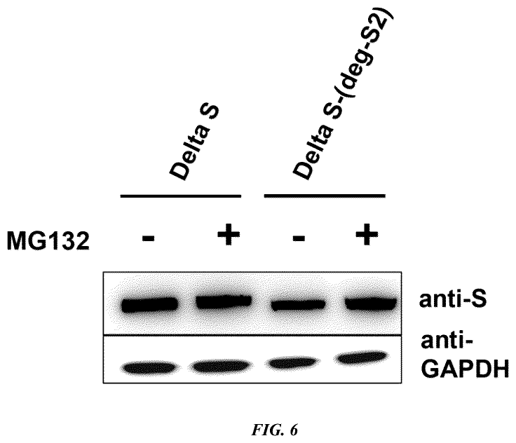

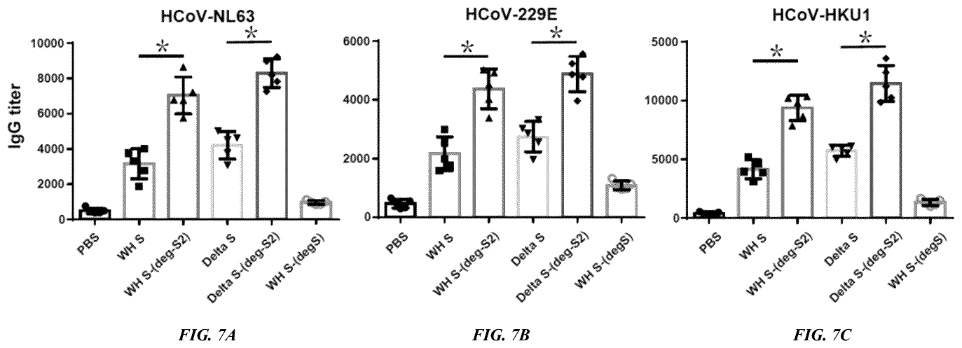

Cross-Reactivities against Alpha and Beta Coronaviruses, Including MERS and SARS Viruses. Lastly, to investigate whether the antibodies induced by the spike mRNA vaccine with deletion of S2 glycosites provide a protection against human alpha and beta coronaviruses since the S2 domain and the stem region contain more conserved epitopes than the other domains among alpha and beta coronaviruses including the strains that cause common cold, SARS-COV-2 variants and MERS as well as SARS virus, it was shown that sera from mice immunized with the S2-glycosite deleted spike mRNA had higher (˜twofold to threefold increase) IgG titers against human alpha [HCoV-NL63 (FIG. 7A) and HCoV-229E (FIG. 7B)] and beta [HCoV-HKU1 (FIG. 7C), HCoV-OC43 (FIG. 7D), MERS-COV (FIG. 7E), and SARS-COV (FIG. 7F)] coronaviruses and increased (˜sixfold to eightfold) neutralization activity against MERS-COV (FIG. 8A) and SARS-COV (FIG. 8B) in pseudovirus neutralization assay, suggesting that the spike mRNA vaccine with deletion of glycosylation on S2 enhances antibody and T cell immune response and provides a broad protection against pan-coronaviruses.

### Conclusion

In the experiments of the present disclosure, 17 conserved epitopes in the SARS-CoV-2 S protein were identified. Among them, 11 of which (more than 60%) are in the S2 region including the six most conserved epitopes in the stem region, and five of the six conserved stem epitopes are also conserved in the stem of SARS-COV, MERS-COV, and other human alpha and beta coronaviruses.

Immunization with spike mRNA vaccine with deletion of S2 glycosites elicited a stronger antibody and T cell response against pan-coronaviruses, suggesting that the induced immune responses target the conserved epitopes in the S2 region. In addition, the WH spike mRNA vaccine with deletion of the six glycosites in the stem region also induced an antibody response with increased IgG titer against the S protein of Delta and Omicron variants, suggesting that the SARS-COV-2 spike mRNA vaccine with deletion of stem glycosylation elicits antibodies that recognized the conserved epitopes in the stem.

Glycosylation on the conserved epitopes of S protein may play an essential role in maintaining the proper tertiary and quaternary structures and simultaneously shielding the conserved epitopes from immune response. The highly conserved epitopes located in the stem region of SARS-COV-2 are also highly conserved among the four coronavirus genera. Therefore, antibodies targeting the conserved epitopes in the stem should provide cross-reactive protection against pan-coronavirus through neutralizing and/or non-neutralizing activity, and deletion of the glycan shields in the highly conserved stem region or the S2 domain to generate low-sugar vaccines should better expose the highly conserved epitopes and elicit enhanced and broadly protective immune responses.

The serum from mice immunized with the SARS-COV-2 S vaccine was overall similar to human convalescent serum, and sera from SARS-COV-2 S2 DNA-vaccinated mice reacted strongly with an epitope in the HR2 and membrane-proximal region that was highly conserved among SARS-COV-2 variants. However, using the recombinant WH S2 protein without glycosylation produced by Escherichia coli failed to induce neutralizing antibodies against SARS-COV-2 WH or Omicron S pseudovirus, perhaps due to the conformation change in vitro. However, the SARS-COV-2 spike mRNA vaccines from the WH and Delta S mRNA with deletion of glycosites in S2 or stem elicited enhanced antibody and CD8⁺ T cell responses against different SARS-COV-2 variants and other coronaviruses, suggesting that the S protein generated in vivo from the mRNA with deletion of glycan shields can be processed to elicit immune responses.

T cell response prevented SARS-COV-2 infection from progressing to severe conditions. In addition, CD8⁺ T cell response induced by prior infection provided approximately 80 to 95% protection against reinfection by SARS-COV-2 variants for more than 8 months. In this study, we have demonstrated that the SARS-COV-2 spike mRNA with deletion of glycosites in the S2 domain or stem region reduced the stability of S protein, thereby triggering a strong memory CD8⁺ T cell induction.

### Additional Material and Methods of Examples A1 to A3

### Cell Lines.

The Human embryonic kidney cells (HEK293) and Huh7 human hepatoma cells were maintained in Dulbecco's modified Eagle's medium (DMEM) (Invitrogen) with 10% heat-inactivated fetal bovine serum (FBS) (Thermo Scientific) and antibiotics (100 U/mL penicillin G and 100 gm/mL streptomycin).

### Antibodies and Proteins.

The rabbit anti-SARS-COV-2 S polyclonal antibody was purchased from ABclonal.

SARS-COV-2 full-length WH and Delta S proteins (293 T cell expressed) were purchased from Royez. HCoV-NL63, HCOV-229E, HCoV-HKU1, and MERS-COV spike protein were purchased from Sino Biologicals. HCoV-OC43 spike protein was obtained from Acrobiosystems. SARS-COV spike protein was purchased from Biotechne. Mouse monoclonal anti-GAPDH was obtained from Millipore. To obtain the deglycosylated S protein, WH or Delta S proteins were deglycosylated in a buffer solution with PNGase F (Sigma) at 37° C. for 24 h in the dark. After deglycosylation, samples were purified and checked by Western blot.

mRNA Vaccine of Deglycosylated S Protein and Formulation.

The prefusion state of the S, the codon-optimized S gene of SARS-COV-2 was synthesized by GenScript and cloned into pVax or pMRNA™, and was stabilized by proline substitutions at positions K968 and V969 (S-2P). To delete the N-glycosites, the putative sequon N-Xa-S/T was changed to Q-Xa-S/T by using site-directed mutagenesis on the S-2P expression plasmid. For the in-vitro transcription, the linear DNA with the T7 promoter, 5′ untranslated region, 3′ untranslated region, S-2P, and poly(A) tail signal sequence was amplified by using TOOLS Ultra High Fidelity DNA Polymerase (BIOTOOLS Co., Ltd.) with 1 μL of the DNA template in an mMESSAGE mMACHINE® Kit (Thermo Scientific) at 37° C. for 1 h according to the manufacturer's protocol. The mRNA was purified by an RNA cleanup kit (BioLabs), according to the manufacturer's protocol, and stored at −80° C. until further use. For the formulation mRNA-LNP, mRNA was encapsulated in LNP using a self-assembly process in which an aqueous solution of mRNA at pH 4.0 was rapidly mixed with an ethanolic lipid mixture containing ionizable cationic lipid, phosphatidylcholine, cholesterol, and polyethylene glycol-lipid. The compositions of LNP were DSPC (Sigma), cholesterol (Sigma), DOTAP (Sigma), and DMG-PEG 2000 (Sigma). The mRNA-LNP was characterized and subsequently stored at −80° C. at a concentration of 1 mg/mL. After HEK293 cells were transfected with 10 μg of mRNA-LNP in six wells of a plate at 48 h, the total cell lysate was collected to monitor the expression of S by Western blot.

### Animals and Immunizations.

BALB/c mice aged 6 to 8 wk (n=5) were immunized intramuscularly with 50 μg mRNA-LNP in PBS with 300 mM sucrose. Animals were immunized at week 0, boosted with a second vaccination at week 2, and serum samples and spleens were collected from each mouse 1 wk after the booster immunization. The animal experiments were evaluated and approved by the Institutional Animal Care and Use Committee of Academia Sinica.

### Serum IgG Titer Measure.

Anti-S protein ELISA was used to determine IgG titer. Plates were coated with 50 ng/well of variant S protein, and then blocked with 5% skim milk. The serum from immunized mice and HRP-conjugated secondary antibodies were sequentially added. Peroxidase substrate solution (TMB) and 1 M H2SO4 stop solution were used and absorbance (OD 450 nm) was read by a microplate reader.

### Pseudovirus Neutralization Assay for Serum Study.

SARS-COV-2 pseudovirus variants were constructed by the RNAi Core Facility at Academia Sinica using the procedure described previously. The pseudotyped lentivirus was then stored at −80° C. To estimate the lentiviral titer by AlarmaBlue assay (Thermo Scientific), the transduction unit (TU) of pseudotyped lentivirus was estimated by using a cell viability assay. HEK-293 T cells expressing the human ACE2 gene were plated on a 96-well plate 1 d before lentivirus transduction. To determine the titer of pseudotyped lentivirus, different amounts of lentivirus were added into the culture medium containing polybrene (final concentration 8 μg/mL) (Sigma), and spin infection was carried out at 1,100×g in 96-well plate for 30 min at 37° C. After incubation for 16 h, the culture medium containing virus and polybrene was removed and replaced with fresh complete DMEM containing 2.5 μg/mL puromycin (Sigma). After treating puromycin for 48 h, the culture medium was removed, and the cell viability was detected by using AlarmaBlue reagents according to the manufacturer's instructions. The survival rate of uninfected cells was set as 100%, and the virus titer was determined by plotting the survival cells versus the diluted viral dose. The SARS-COV and MERS-COV pseudoviruses were purchased from eEnzyme.

For neutralization assay, heat-inactivated sera or antibodies were serially diluted with desired dilution and incubated with 1,000 TU of SARS-COV-2 pseudotyped lentivirus in DMEM for 1 h at 37° C. The mixture was then inoculated with 10,000 HEK-293 T cells stably expressing the human ACE2 gene or Huh7 (for MERS-COV pseudovirus) in a 96-well plate. The culture medium was replaced with fresh complete DMEM (supplemented with 10% FBS and 100 U/mL Penicillin/Streptomycin) at 16 h postinfection and continuously cultured for another 48 h. The expression level of the luciferase gene was determined by using Bright-Glo™ Luciferase Assay System (Promega). The relative light unit (RLU) was detected by Tecan i-control (Infinite 500). The percentage of inhibition was calculated as the ratio of RLU reduction in the presence of diluted serum to the RLU value of no serum control and the calculation formula as shown below: (RLU^{control}−RLU^{Serum})/RLU^{control}.

### DNA Plasmid Transfection and MG132 Treatment.

After the HEK293 cell was seeded in the six-well plates, cells were transfected with 3 μg of each plasmid by TransIT®-LT1 Transfection Reagent (Mirus) and then incubated with 1 μM MG-132 (MedChemExpress) or dimethyl sulfoxide (DMSO) at 37° C. for 24 h. The total lysate was collected and the variant S expression was analyzed by Western blot.

### Statistics and Reproducibility.

All data were presented as means±SEM. The numbers of samples and replicates of experiments were shown as mentioned in the figure legends. Comparisons between groups were determined using the Student's t test. Differences were considered significant at *P<0.001, **P<0.05. All data were analyzed using GraphPad Prism 6 software.

### Example B1: Synthesis of Exemplary Compounds of the Present Disclosure

### Chemical Materials and Methods

For chemical synthesis, all starting materials and commercially obtained reagents were purchased from Sigma-Aldrich and used as received unless otherwise noted. All reactions were performed in oven-dried glassware under a nitrogen atmosphere using dry solvents. ¹H and ¹³C NMR spectra were recorded on Brucker AV-600 spectrometer, and were referenced to the solvent used (CDCl₃ at δ 7.24 and 77.23, CD₃OD at δ 3.31 and 49.2, and D₂O at δ 4.80, and DMSO-d₆ at δ 2.5 and 39.51 for ¹H and ¹³C, respectively). Chemical shifts (δ) are reported in ppm using the following convention: chemical shift, multiplicity (s=singlet, d=doublet, t=triplet, q=quartet, m=multiplet), integration, and coupling constants (J), with J reported in Hz. High-resolution mass spectra were recorded under ESI-TOF mass spectroscopy conditions. Silica gel (E, Merck) was used for flash chromatography. IMPACT™ system (Intein Mediated Purification with Affinity Chitinbinding Tag) was purchased from New England Biolabs. His-tag purification resin was purchased from Roche. HiTrap IMAC column (5 mL) was purchased from GE Healthcare Life Sciences. Gel permeation chromatography (GPC) equipped with Ultimate 3000 liquid chromatography associated with a 101 refractive index detector and Shodex columns was used to analyze the polymeric products using THE as the eluent at 30° C. with 1 mL min⁻¹ flow rate. The calibration was based on the narrow linear poly(styrene) Shodex standard (SM-105). The Mw and dispersity of the polymeric products were calculated using DIONEX chromeleon software. Transmission electron microscopy (TEM) images were obtained by a FEI Tecnai G2 F20 S-Twin.

The chemical materials and methods described herein apply to all examples described in the present disclosure.

### Synthesis and Results

The exemplary compounds described here were synthesized according to the synthesis Scheme 1, Scheme 2, and Scheme 3 below. The detailed synthesis procedures are described below.

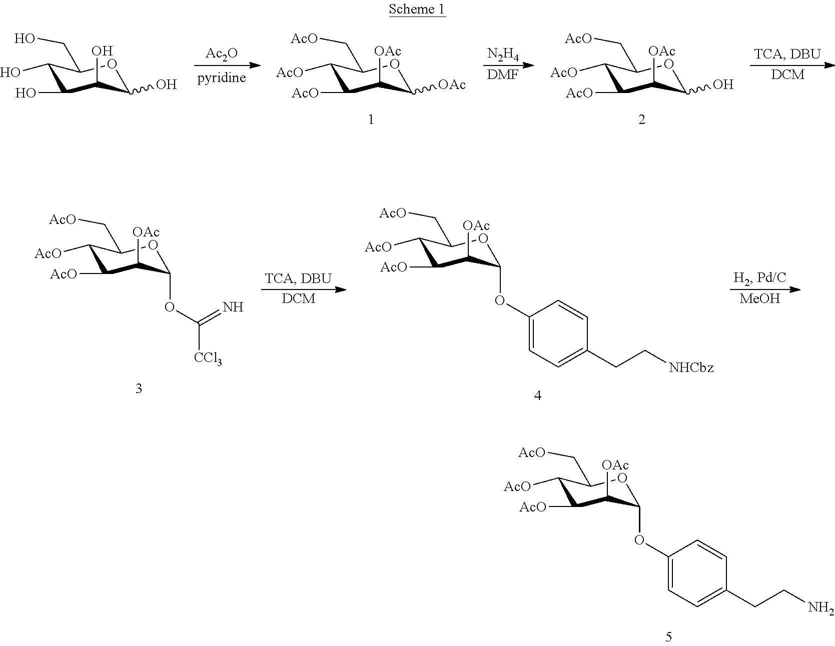

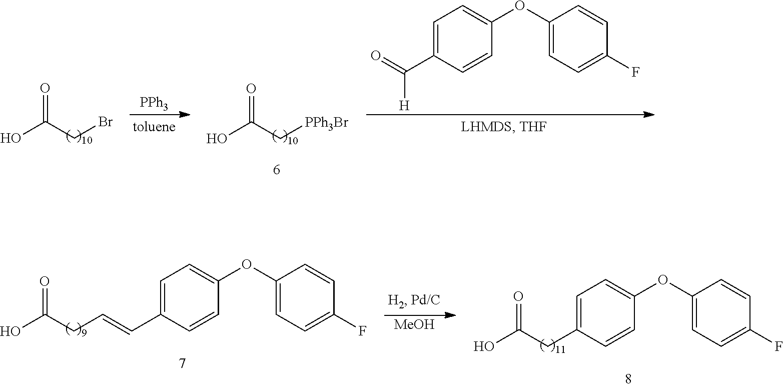

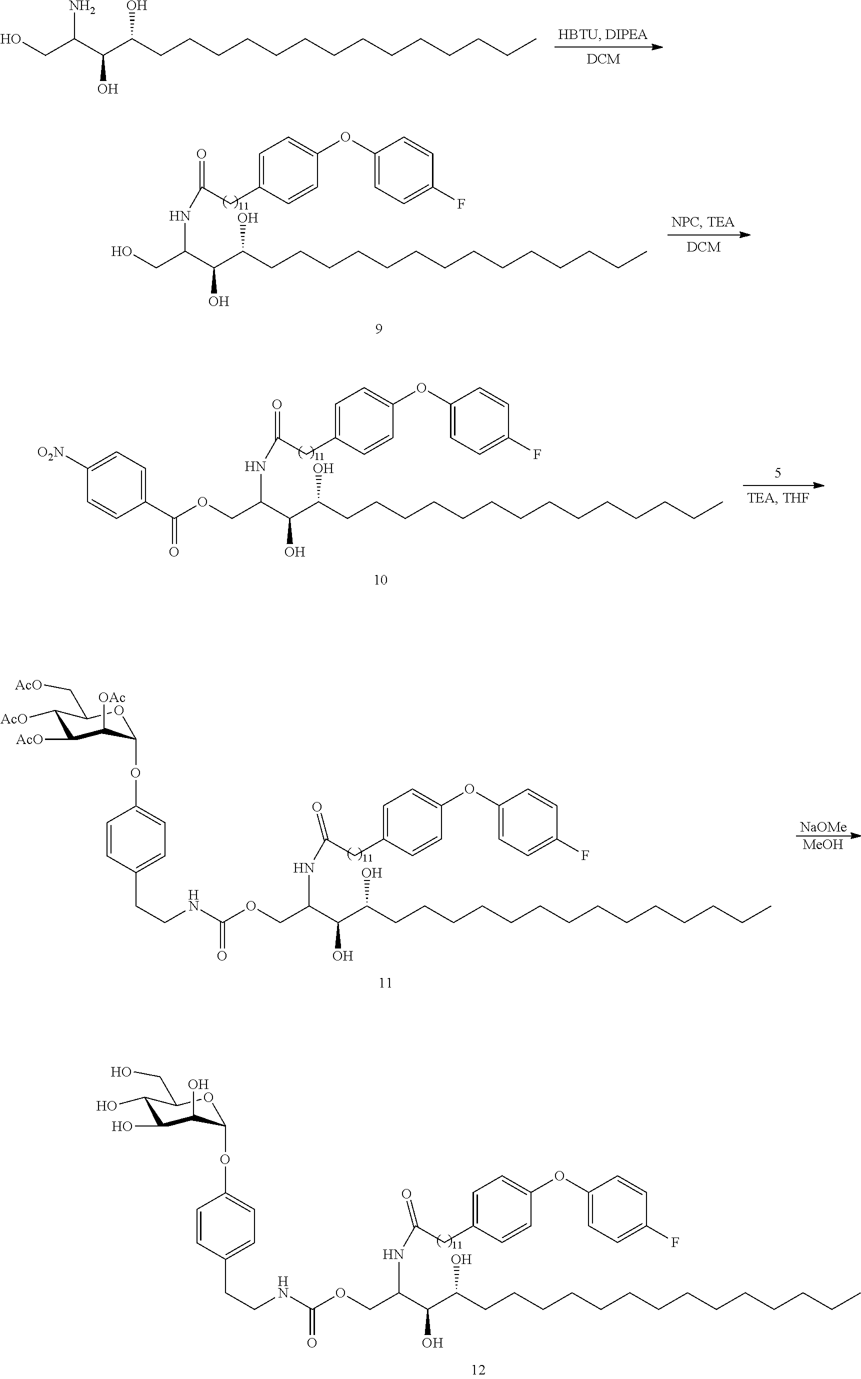

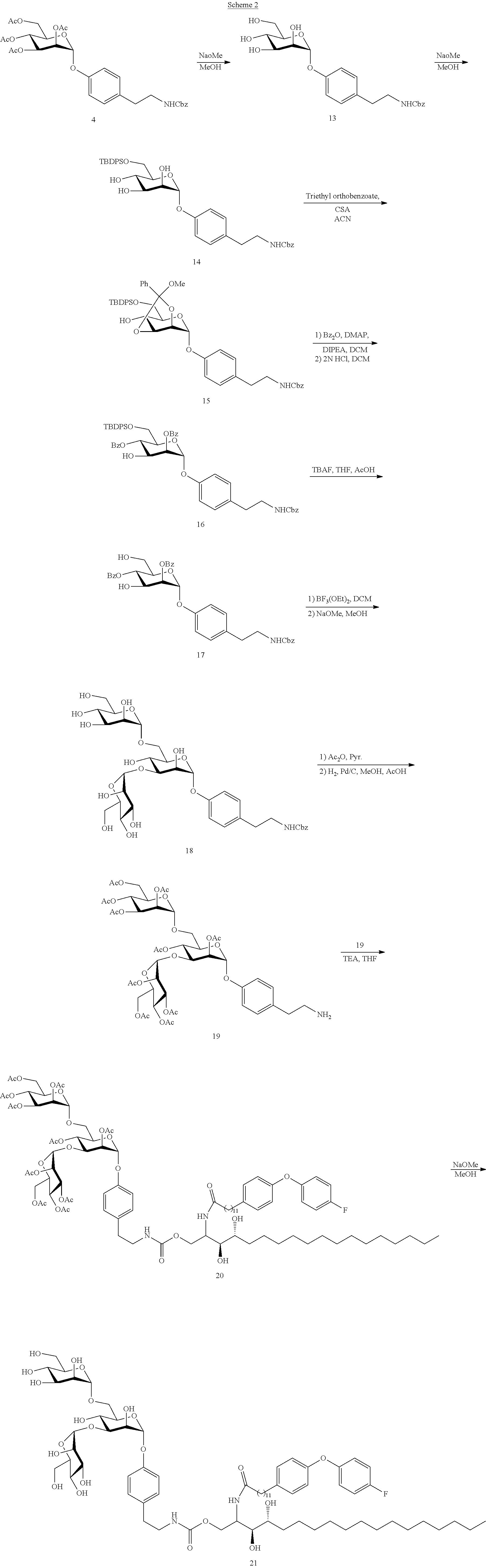

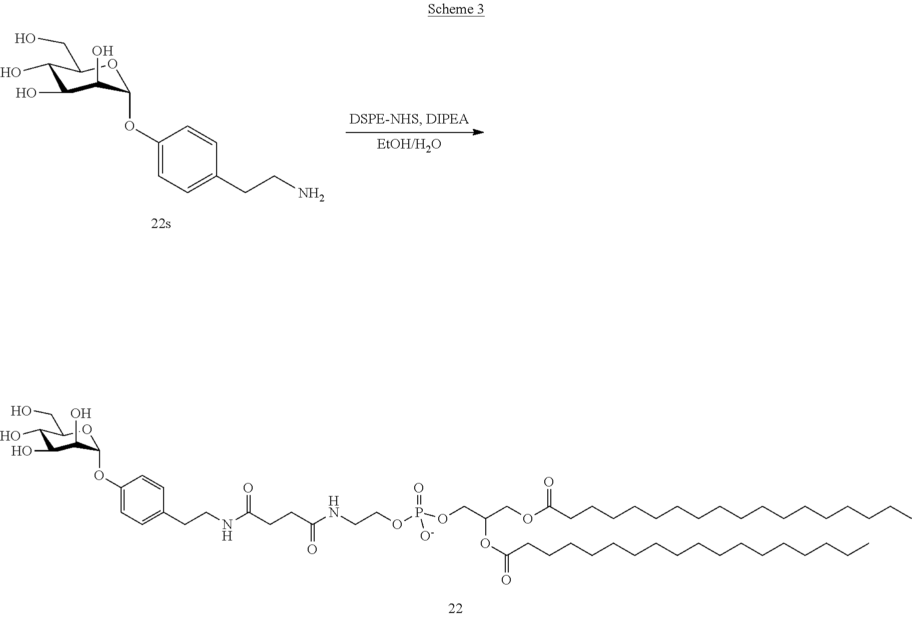

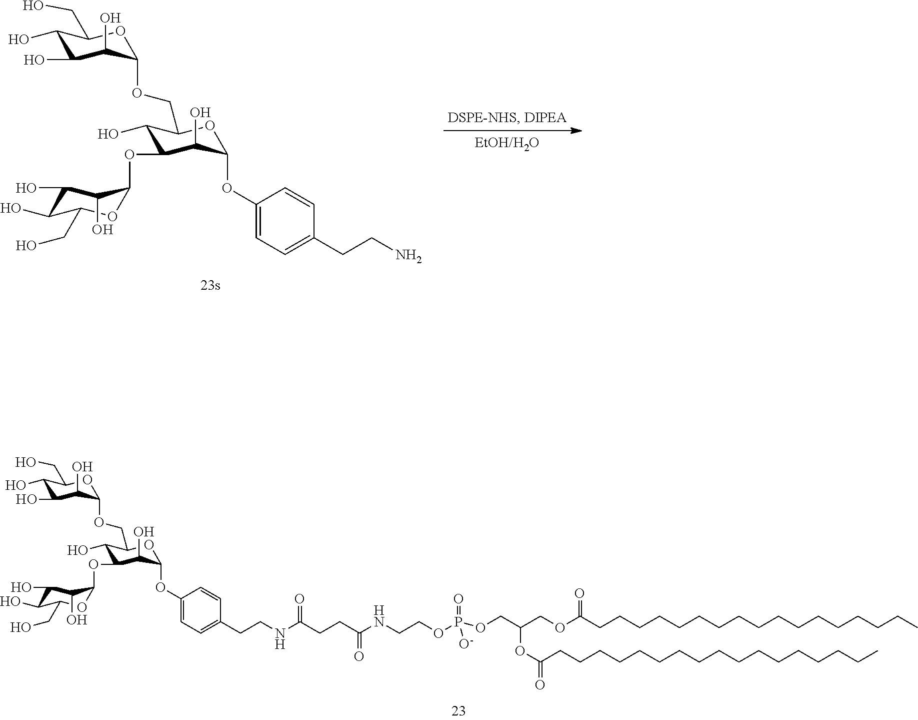

### Compounds 1 to 5

Compounds 1-5 were synthesized and characterized according to a published protocol (ACS Nano 2021, 15, 309-321).

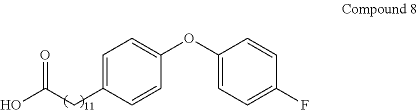

(11-Carboxynonyl)triphenylphosphonium bromide 6 (2.5 g, 10 mmol) was prepared by refluxing triphenylphosphine (10 mmol) and 11-bromoundecanoic acid (10 mmol). It was then dissolved in 50 ml of tetrahydrofuran (THF) and cooled to 0° C. lithium bis(trimethylsilyl)amide (LHMDS; 1 M in THF, 20 mmol) was added to the solution to produce an orange ylide. After that, 4-(4-Fluorophenoxy)benzaldehyde (12 mmol) in 20 ml of THF was added dropwise to the solution and stirred for 4 h at room temperature. The reaction was quenched with methanol and concentrated. The residue was extracted with EA and brine and then dried over MgSO₄. After removal of the solvent, the mixture was chromatographed on silica gel (EA-Hex=1:2) to give the unsaturated fatty acid 7. The saturated fatty acid was prepared by catalytic hydrogenation in 50 ml of methanol containing 10 mol % of 10% palladium on charcoal (Pd/C). The reaction mixture was stirred under H₂ at room temperature overnight. The hydrogenated product was filtered through Celite and the resulting solution was concentrated and chromatographed on silica gel (EA-Hex=1:2) to give the product as a yellow solid (66%).

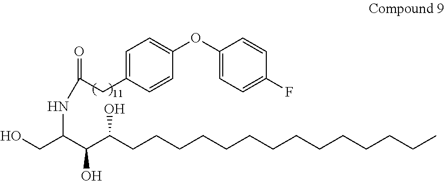

Compound 9. Compound 8 (1 mmol) in THF (10 mL) was added EDC (1.5 mmol), HOBt (1.5 mmol), DMAP (0.1 mmol), trimethylamine (2 mmol), and phytosphingosine (1.2 mmol), and the resulting solution was stirred under nitrogen at rt for 12 h. The solvent was then removed by evaporation, followed by extraction with EA/H₂O. The collected organic layer was washed with saturated NaHCO₃ (aq), water and brine, and dried over MgSO₄. The crude product was purified by column chromatography on silica gel (EA/Hex 1:1) to yield 9 (74%).

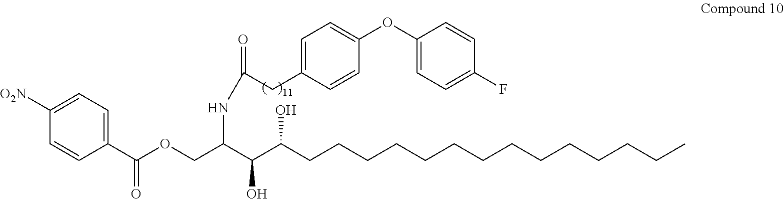

Compound 9 (1 mmol) in THF (10 mL) was added 4-nitrophenylchloroformate (2 mmol), trimethylamine (2 mmol), and the resulting solution was stirred under nitrogen at rt for 12 h. The solvent was then removed by evaporation, and the crude compound was directly used for the next step without further purification.

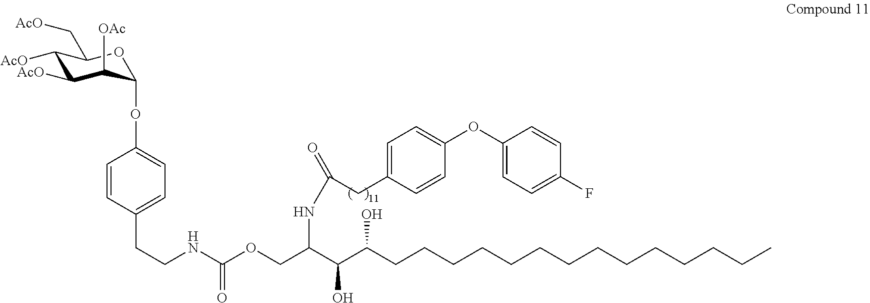

Compound 5 (1 mmol) in THF (10 mL) was added 10 (1 mmol) and trimethylamine (2 mmol), and the resulting solution was stirred under nitrogen at rt for 2 h. The solvent was then removed by evaporation, followed by extraction with EA/H₂O. The collected organic layer was washed with saturated NaHCO₃ (aq), water and brine, and dried over MgSO₄. The crude product was purified by column chromatography on silica gel (EA/Hex 1:1+10% MeOH) to yield 11 (59%).

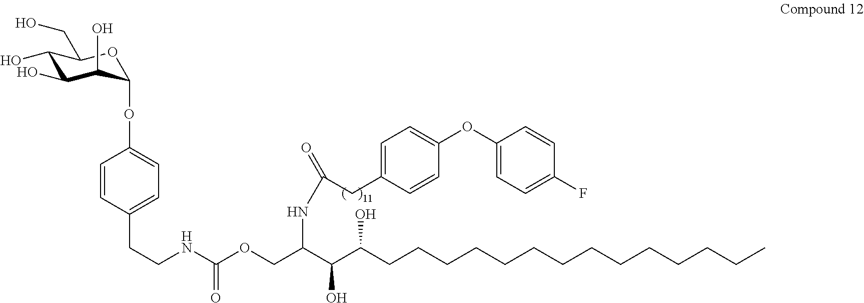

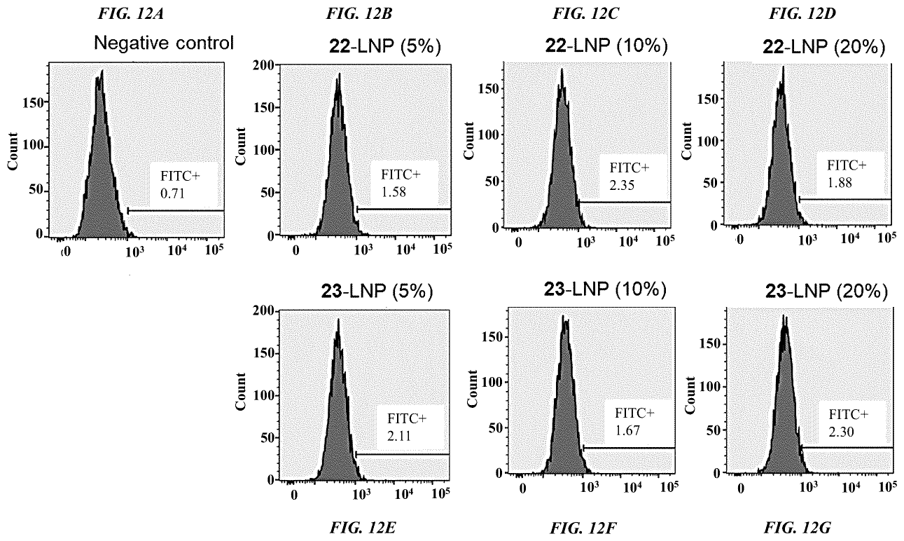

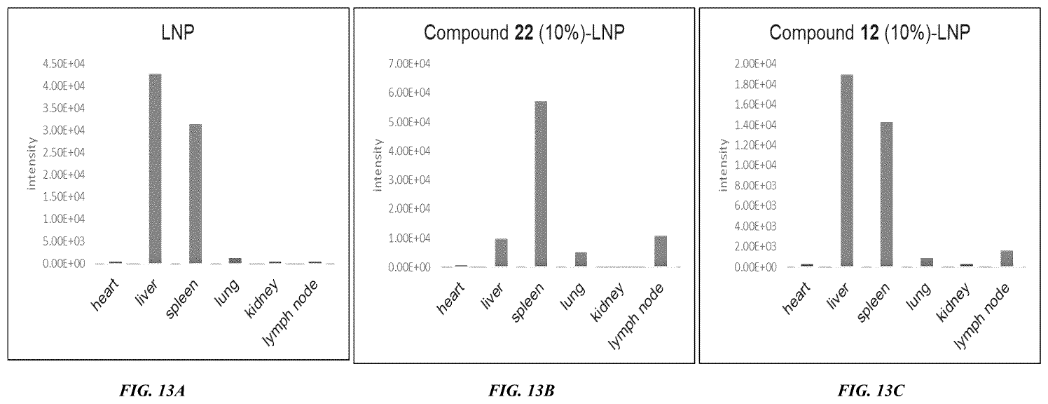

Compound 11 in MeOH was added NaOMe (0.2 eq), and the resulting solution was stirred under nitrogen at room temperature for 2 hours. The mixture was neutralized by IR-120 and then filtered and concentrated to dryness in vacuo to give compound 12 (quant.) (FIG. 14 and FIG. 15).

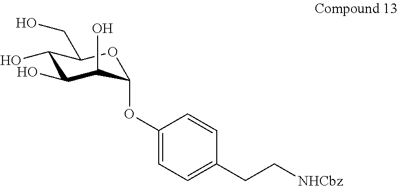

Compound 4 in MeOH was added NaOMe (0.2 eq), and the resulting solution was stirred under nitrogen at rt for 2 h. The mixture was neutralized by IR-120 and then filtered and concentrated to dryness in vacuo to give compound 13 (quant.).

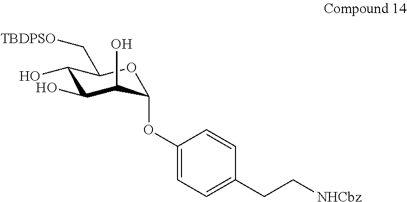

Compound 13 (1 mmol) in MeOH was added NaOMe (0.2 eq), and the resulting solution was stirred under nitrogen at rt for 2 h. The mixture was neutralized by IR-120 and then filtered and concentrated to dryness in vacuo. It was then dissolved in anhydrous DCM (10 mL) and treated with imidazole (1.5 mmol) at 0° C., followed by the addition of TBDPSCl (1.2 mmol). The mixture was stirred at room temperature for 2.5 h under a nitrogen atmosphere. The reaction was quenched by the addition of MeOH. After stirring at room temperature for 10 min, the solvent was removed under reduced pressure to give a dry residue that was purified by column chromatography with MeOH/DCM (1/10) to give compound 14 (82%).

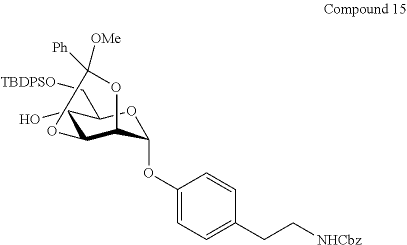

To a solution of compound 14 (1 mmol) and a catalytic amount of CSA (0.1 mmol) in CH₃CN (20 mL) was added trimethyl orthobenzoate (3 mmol) at room temperature under atmospheric pressure of nitrogen. After stirring for 30 min, Et₃N was added to quench the reaction, and the resulting mixture was dried under reduced pressure. The residue was purified by column chromatography with EA/Hex (1/2) to give compound 15 (79%).

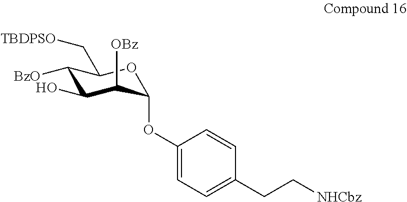

Compound 15 (1 mmol) was dissolved in DCM (10 mL) and sequentially mixed with DIPEA (2 mmol), benzoic anhydride (2 mmol), and DMAP (0.1 mmol). After stirring for 2 hr, the solvent was evaporated under reduced pressure to give a dry residue and then poured into EA (20 mL) and 2 N HCl (10 mL) with vigorous stirring for 30 min. The solvent was then removed by evaporation, followed by extraction with EA/H₂O. The collected organic layer was washed with ice-cold saturated NaHCO₃ (aq), water and brine, and dried over MgSO₄. The dry residue was purified by column chromatography with EA/Hex (1/2) to give compound 16 (71%).

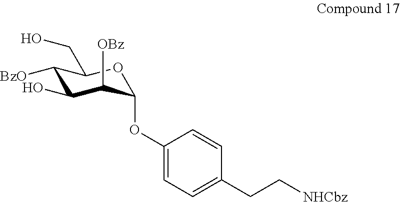

Compound 16 (1 mmol) was added AcOH (4 mmol) and 1 M TBAF (2.4 mmol in THF) at 0° C. The resulting mixture was warmed up to room temperature gradually, stirred for another 2 h, and then diluted with EA. The organic layer was washed with saturated NaHCO₃ (aq), water, and brine, dried with anhydrous MgSO₄, and concentrated under reduced pressure. The dry residue was purified by column chromatography with EA/Hex (1/2) to give compound 47 (88%).

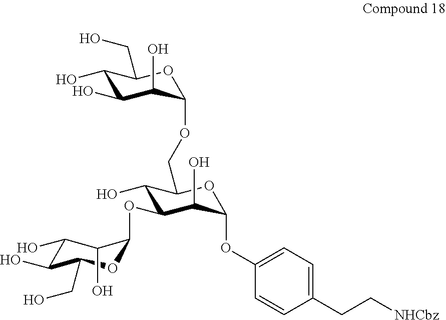

To a stirred solution of 17 (1 mmol) and 4 Å molecular sieve (0.1 g) in anhydrous DCM (10 mL) was cooled to −40° C. and then BF₃(OEt)₂ (0.1 mmol) was added dropwise to the solution. A solution of 3 in anhydrous DCM was added dropwise to the above mixture and stirred for 1 h at −40° C. After that, the reaction was gradually warmed to room temperature and stirred for another 1 h. The solution was quenched by adding triethylamine, then filtered and added saturated. NaHCO₃ aq. and extracted with DCM. The organic layer was dried with MgSO₄ and evaporated to dryness. The residue was purified by flash column chromatography on silica gel to give a trisaccharide product. The product was then dissolved in MeOH, and NaOMe (0.2 eq) was added, and the resulting solution was stirred at room temperature for 2 h. The mixture was neutralized by IR-120 and then filtered and concentrated to dryness in vacuo. The deacetylated mixture was purified by Bio-Gel P-2 Gel (Biorad) with H₂O as eluent to obtain a pure trisaccharide. The compound was lyophilized to dryness to give compound 18 (39%).

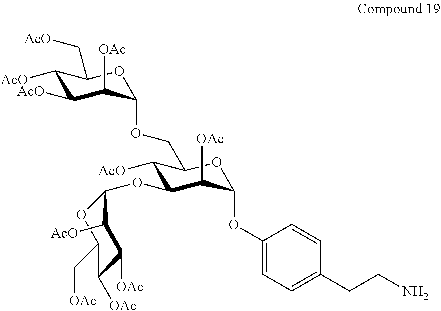

Compound 13 (1 mmol) in MeOH was added NaOMe (0.2 eq), and the resulting solution was stirred under nitrogen at rt for 2 h. The mixture was neutralized by IR-120 and then filtered and concentrated to dryness in vacuo.

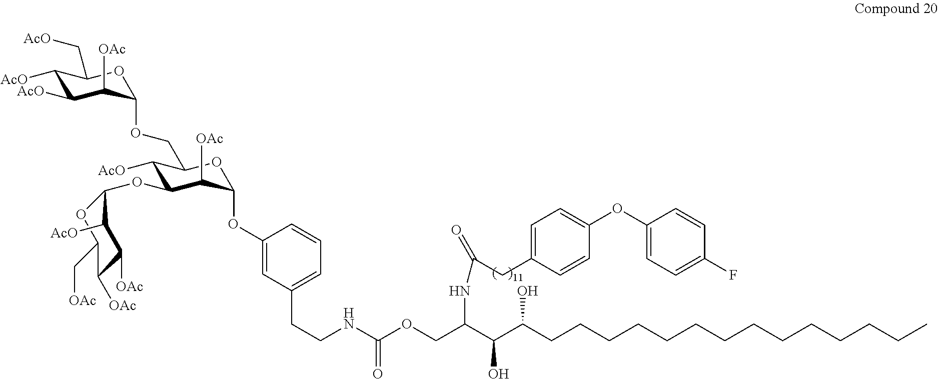

Compound 19 (1 mmol) in THF (10 mL) was added 10 (1 mmol) and trimethylamine (2 mmol), and the resulting solution was stirred under nitrogen at rt for 2 h. The solvent was then removed by evaporation, followed by extraction with EA/H₂O. The collected organic layer was washed with saturated NaHCO₃ (aq), water and brine, and dried over MgSO₄. The crude product was purified by column chromatography on silica gel (EA/Hex 1:1+10% MeOH) to yield 20.

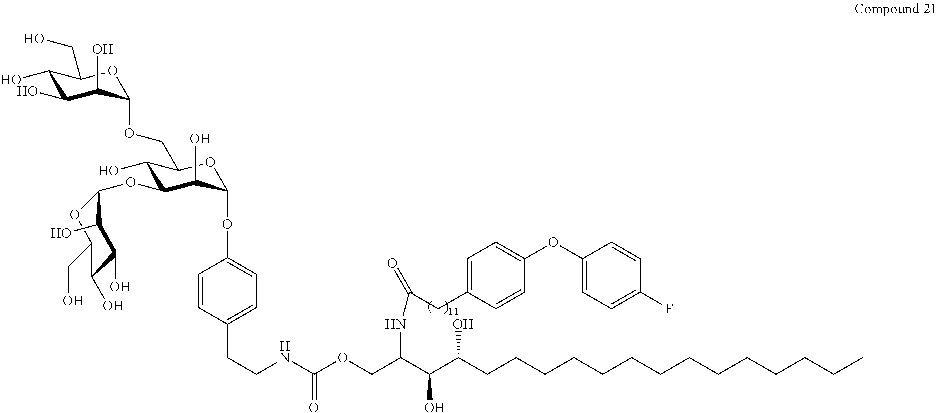

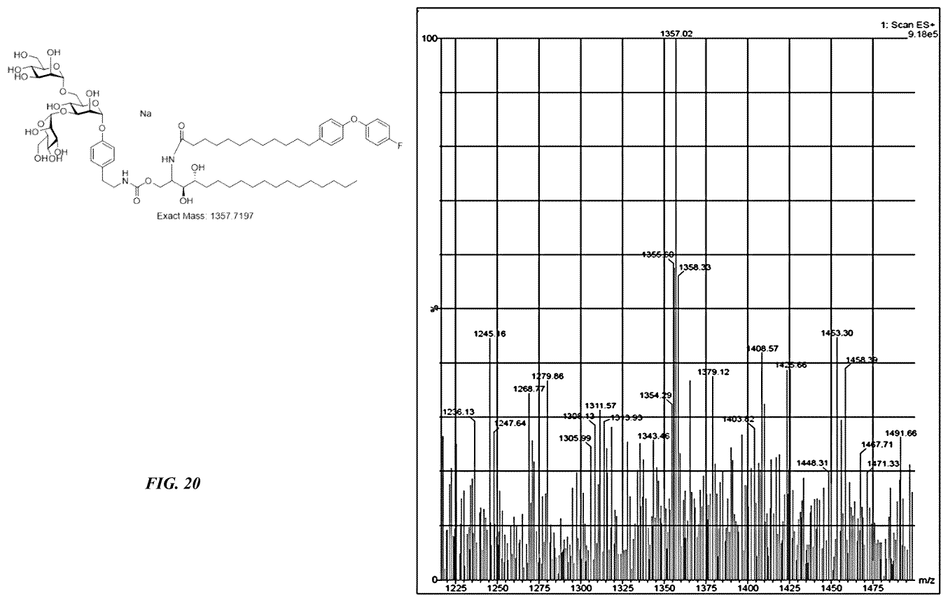

Compound 20 in MeOH was added NaOMe (0.2 eq), and the resulting solution was stirred under nitrogen at room temperature for 2 hours. The mixture was neutralized by IR-120 and then filtered and concentrated to dryness in vacuo to give compound 21 (quant.). The resulting compound 21 was examined using LCMS spectrum, which shows peaks at 1236.13, 1245.16, 1247.64, 1268.77, 1279.86, 1305.99, 1308.13, 1311.57, 1313.93, 1343.46, 1354.29, 1355.60, 1358.33, 1379.12, 1403.82, 1408.57, 1425.66, 1448.31, 1453.30, 1458.39, 1467.71, 1471.33, and 1491.66 (FIG. 20).

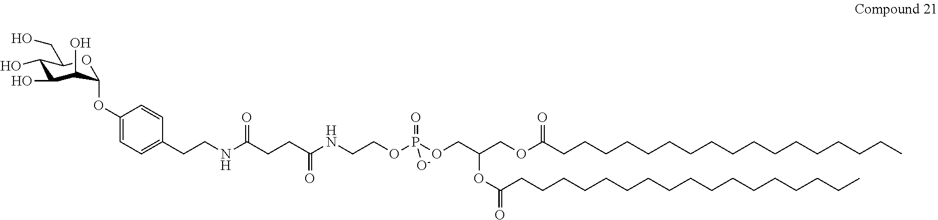

Arylmannoside 22s (0.1 mmol) in EtOH/H₂O (0.5/0.5 mL) was added to DSPE-NHS (0.1 mmol), and trimethylamine (2 mmol), and the resulting solution was stirred at rt for 12 h. The solvent was removed by evaporation and the crude product was purified Bio-Gel P-2 Gel with H₂O as eluent to yield 22 (79%).

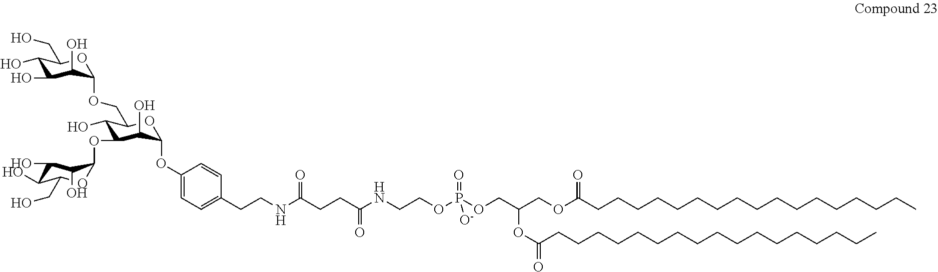

Aryltrimannoside 23s (0.1 mmol) in EtOH/H₂O (0.5/0.5 mL) was added DSPE-NHS (0.1 mmol) and trimethylamine (2 mmol), and the resulting solution was stirred at room temperature for 12 h. The solvent was removed by evaporation and the crude product was purified Bio-Gel P-2 Gel with H₂O as eluent to yield 23 (76%).

### Example B2: Preparation and Characterization of the LNP of the Present Disclosure

### Preparation of LNP

A lipid mix solution in EtOH (10 mg/ml) having a molar ratio of 50% SM-102, 10% DSPC, 38.5% cholesterol, and 1.5% DMG-PEG2000 was prepared. An LNP formulation was prepared by mixing the compound of the present disclosure with the lipid mix solution (with a molar ratio of 45% SM-102, 9% DSPC, 34.5% cholesterol, 1.5% DMG-PEG2000, and 10% compound of the present disclosure). The LNP formulation was added into a 1.5 mL tube. Then, a mRNA payload, diluted with citrate buffer before use (10 mM, pH4), was added to the tube at a final concentration of 0.18 ug/uL. The mRNA aqueous solution in the tube was then quickly added to an ethanol solution and mixed well by vortex for 1 minute. The resulting solution was then dialyzed by micro float-A-Lyzer (8-10 kD) against PBS at 4° C. overnight to obtain the LNP of this example. The resulting LNP can be stored at 4° C. for a few days before use.

### Characterization of LNP

Size Measurement. The LNP prepared above was examined using dynamic light scattering (DNP) to measure its size. First, 5 L of the LNP solution was transferred to a clean 1.5 mL tube and diluted with 95 μL of PBS. The mixture was then transferred to a cuvette, and the particle size of the LNP was measured using a Nano ZS machine. The following table shows the sizes and the Polydispersity Index (PDI) of the LNP samples prepared.

TABLE
the size and PDI measurement

| | Size (nm)_Mean ± | |
|---|---|---|
| Sample | SD | PDI_Mean ± SD |
| Compound 24- | 138.5 ± 0.7074 | 0.1498 ± 0.002694 |
| LNP | | |
| Compound 25- | 161 ± 0.3246 | 0.1221 ± 0.02095 |
| LNP | | |
| Compound 12- | 177.5 ± 1.79 | 0.1381 ± 0.02234 |
| LNP | | |
| Compound 22- | 191.7 ± 1.617 | 0.1625 ± 0.0294 |
| LNP | | |
| Compound 23- | 170.7 ± 1.353 | 0.1109 ± 0.01918 |
| LNP | | |

Zeta potential and encapsulation efficiency. Next, the encapsulation efficiency of the LNP of the present disclosure was evaluated using a Quant-it Ribogreen assay. A 2000-fold diluted quant-it Ribogreen reagent with 1×TE (working solution) was prepared. Then, an RNA standard dilution series from 0-50 ng/ml (100 μL) was prepared to obtain a standard curve. 5 μL of the LNP solution prepared above was transferred to a clean tube and diluted to a final volume of 100 μL. The working solution of the quant-it Ribogreen reagent (100 μL) was then added to the LNP sample. The fluorescence signal of the sample was then detected using a microplate reader (ex/em 485/535). According to the standard curve, the fluorescence signal was used to calculate the concentration of unencapsulated mRNA in solution (ng/mL). For zeta potential measurement, 0.75 mL DP-intermediate was introduced into capillary cells and measured at 25° C. using Malvern Zetasizer Pro equipment.

TABLE
Zeta potential and encapsulation efficiency

| | | Encapsulation |
|---|---|---|
| Sample | Zeta potential (mV) | efficiency (%) |
| Compound 24- | 0.113 | 92.15 |
| LNP | | |
| Compound 25- | −4.382 | 86.69 |
| LNP | | |
| Compound 12- | 0.932 | 81.47 |
| LNP | | |
| Compound 22- | −0.5107 | 82.86 |
| LNP | | |
| Compound 23- | 0.3875 | 90.00 |
| LNP | | |

### Example B3: In Vitro Uptake and Transfection of mRNA-LNPs in Dendritic Cells

### Experiment 3-1

Splenic cell preparation and BMDC culture. This example tested the uptake of several exemplary LNPs (as shown in the table below) according to the embodiments of the present disclosure in bone marrow-derived dendritic cells (BMDCs) and splenic cells. To prepare splenic cells, the mouse spleen was homogenized with the frosted end of a glass slide and treated with RBC lysis buffer (Sigma) to deplete red blood cells (RBCs), followed by passing through the cell strainer (BD Biosciences). Bone marrow was isolated from mouse femurs and tibiae and treated with RBC lysis buffer (Sigma-Aldrich) to deplete RBCs. Cells were then cultured in RPMI-1640 containing 10% heat-inactivated FBS (Thermo Fisher Scientific), 1% Penicillin/Streptomycin (Thermo Fisher Scientific), 50 μM 2-mercaptoethanol (Thermo Fisher Scientific), and 20 ng/ml recombinant mouse GM-CSF (eBioscience) at a density of 2×10⁵ cells/ml. The cells were supplemented with an equal volume of the complete culture medium (RPMI-1640, 100 U/ml Pen/Strep, 55 μM 2-mercaptoethanol, and 10% FBS) at day 3 and refreshed with one-half the volume of the medium at day 6. On day 8, the suspended cells were harvested.

Table of the exemplary LNPs tested in this experiment.

| | | | | | | L5 |
|---|---|---|---|---|---|---|
| | L1: | L2: | | | | the compound |
| | Ionizable | Phosphatidyl- | | L3: | L4: | of the present |
| Example | lipid | choline | | Cholesterol | PEG-Lipid | disclosure |
| 1 | 45 mol % | 9 | mol % | 34.5 mol % | 1.5 mol % | Compound |
| | | | | | | 24 |
| | | | | | | 10 mol % |
| 2 | 45 mol % | 9 | mol % | 34.5 mol % | 1.5 mol % | Compound |
| | | | | | | 25 |
| | | | | | | 10 mol % |
| 3 | none | none | | none | none | none |
| N.C. | | | | | | |
| (no LNP) | | | | | | |
| 4 | 50 mol % | 10 | mol % | 38.5 mol % | 1.5 mol % | none |

P.C.

Treatment of LNPs to splenic cells and BMDCs. Splenic cells or BMDCs were incubated with different FITC-labeled LNP formulations in RPMI-1640 at 37° C. for 1 hour. Cells were blocked with an Fc receptor binding inhibitor (clone: 93, eBioscience) for 20 minutes. Splenocytes were stained with antibodies against CD3 (clone: 17A2, BV421-conjugated, Biolegend), CD19 (clone: 1D3, PECy7-conjugated, BD Biosciences). BMDCs were stained with antibodies against CD11c (clone N418 APC-conjugated, Biolegend). Labeled cells were analyzed using FACSC and Flow Cytometer (BD Biosciences).

Flow Cytometry. After incubation with different mRNA-LNPs, BMDC cells were washed with ice-cold FACS buffer (1% FBS in 1×DPBS with 0.1% Sodium Azide), and incubated with purified anti-mouse CD16/32 antibody (BioLegend) in FACS buffer on ice for 20 min, followed by washing with FACS buffer. BMDCs were stained with APC anti-mouse CD11c antibody (BioLengend) at 4° C. for 30 min, and washed with FACS buffer. Finally, BMDCs were stained with propidium iodide (Sigma-Aldrich). Flow cytometry was performed on a FACS Canto™ flow cytometer (BD Bioscience).

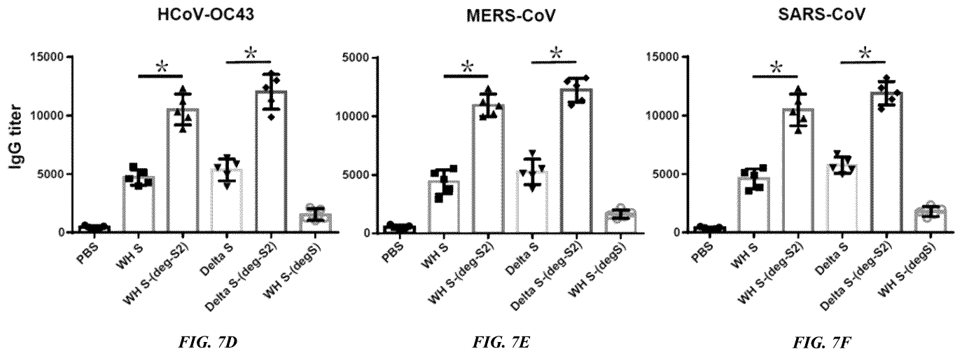

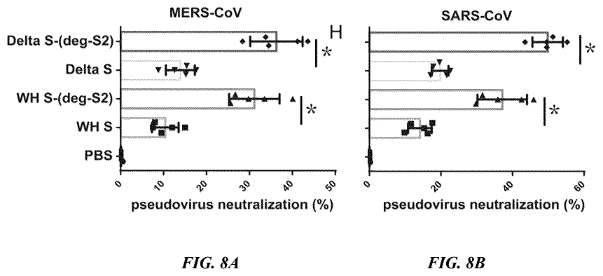

Results. The FACS results are shown in FIGS. 9A-9H and FIGS. 10A-10L and the table below. FIG. 9A, FIG. 9B, FIG. 9C, FIG. 9D, FIG. 9E, FIG. 9F, FIG. 9G, and FIG. 9H show that the BMDCs showed specific uptake of the LNPs made using compounds of the present disclosure compared with non-BMDCs. A traditional LNP (i.e., without using the compound of the present disclosure) showed slightly higher uptake by the BMDCs than the non-BDMCs, but the specificity was insignificant compared to that of the LNPs of the present disclosure (56.1/8.62 or 55.2/6.64 vs. 0.48/0.12). Similarly, in FIG. 10A, FIG. 10B, FIG. 10C, FIG. 10D, FIG. 10E, FIG. 10F, FIG. 10G, FIG. 10H, FIG. 10I, FIG. 10J, FIG. 10K, and FIG. 10L, dendritic cells (DCs) showed specific uptake of the LNPs of the present disclosure at least 3 times higher than B cells (30.6/10.7 and 38.3/11.9) and at least 6 times higher than T cells (30.6/0.5 and 38.3/0.68). DCs showed higher uptake of traditional LNPs but the inclination was less significant than that of the LNPs of the present disclosure.

Table of the uptake results (arbitrary unit of the FITC signals).

Table of the uptake results (arbitrary unit of the FITC signals)

| | L5 the | | | | | |
|---|---|---|---|---|---|---|
| | compound of | FITC signal (A.U.) | | | | |
| Experiment | the present | | Non- | | B | T |
| 3-1 | disclosure | BMDC | BMDC | DCs | cells | cells |
| Example 1 | Compound 24 | 56.1 | 8.62 | 30.6 | 10.7 | 0.50 |
| Example 2 | Compound 25 | 55.2 | 6.64 | 38.3 | 11.9 | 0.68 |
| Example 3 | Negative | 0.17 | 0.18 | 0.26 | 0.016 | 0.022 |
| | control | | | | | |
| Example 4 | Positive | 0.48 | 0.12 | 16.9 | 1.46 | 0.26 |
| | control | | | | | |

### Experiment 3-2

Exemplary LNPs (as shown in the table below) made using different formulations according to the embodiments of the present disclosure were tested in this experiment. Both uptake and transfection were tested to assess whether the payload delivered by the LNPs of the present disclosure can be expressed properly in targeted cells. Bone marrow-derived dendritic cells (BMDCs) were isolated from murine tibia and femurs of 57BL/6 mice. Bone marrow cells were stimulated for 8 days with 20 ng/mL GM-CSF in RPMI medium (RPMI-1640, 100 U/ml Pen/Strep, 55 uM 2-mercaptoethanol and 10% FBS). After 8 days of culture, 1×10⁶ BMDCs (centrifuge 400 g, 5 mins and replace medium with 1 ml Opti-MEM) were plated in 6-well plates, and different samples of LNPs encapsulating mRNA were diluted by 0.25 mL Opti-MEM and incubated with BMDC.

For uptake analysis, FITC-labelled LNPs encapsulating mRNA that encodes a SARS COV2 Spike protein were incubated with the BMDCs at 37° C. for 2 hours. For transfection analysis, the LNPs encapsulating eGFP mRNA were incubated with the BMDCs at 37° C. for 4 hours. After 4 hours of transfection, BMDCs were supplemented with the 1.25 ml complete RPMI medium and incubated at 37° C. for 48 hours. The experiments were conducted using FACS, similar to that described above.

Table of the exemplary LNPs tested in this experiment.

| | | | | | | | L5 |
|---|---|---|---|---|---|---|---|
| | L1: | | L2: | | | | the compound |
| | Ionizable | | Phosphatidyl- | | L3: | L4: | of the present |
| Example | lipid | | choline | | Cholesterol | PEG-Lipid | disclosure |
| 1 | 47.5 | mol % | 9.5 | mol % | 36.5 mol % | 1.5 mol % | 5 mol % |
| | | | | | | | Compound |
| | | | | | | | 22 |
| 2 | 45 | mol % | 9 | mol % | 34.5 mol % | 1.5 mol % | 10 mol % |
| | | | | | | | Compound |
| | | | | | | | 22 |
| 3 | 40 | mol % | 8 | mol % | 30.5 mol % | 1.5 mol % | 20 mol % |
| | | | | | | | Compound |
| | | | | | | | 22 |
| 4 | 47.5 | mol % | 9.5 | mol % | 36.5 mol % | 1.5 mol % | 5 mol % |
| | | | | | | | Compound |
| | | | | | | | 23 |
| 5 | 45 | mol % | 9 | mol % | 34.5 mol % | 1.5 mol % | 10 mol % |
| | | | | | | | Compound |
| | | | | | | | 23 |
| 6 | 40 | mol % | 8 | mol % | 30.5 mol % | 1.5 mol % | 20 mol % |
| | | | | | | | Compound |
| | | | | | | | 23 |
| 7 | 50 | mol % | 10 | mol % | 38.5 mol % | 1.5 mol % | none |

(control)

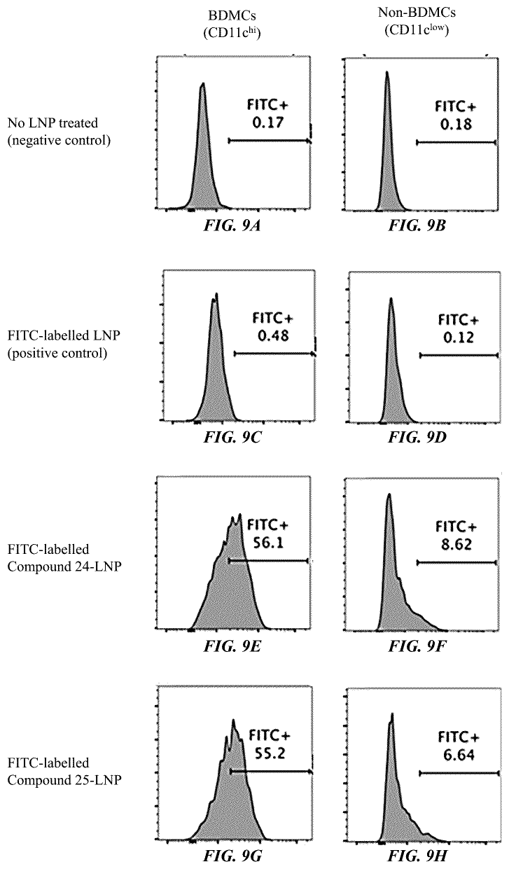

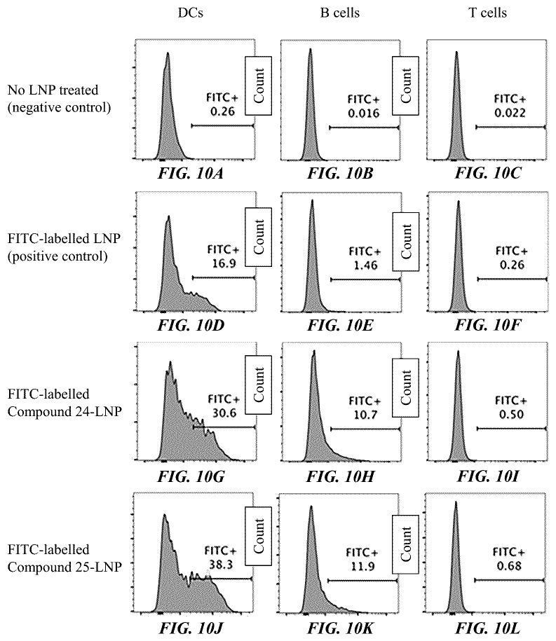

Results. The FACS results are shown in FIGS. 11A-11G and FIGS. 12A-12G and the table below. FIG. 11A, FIG. 11B, FIG. 11C, FIG. 11D, FIG. 11E, FIG. 11F, and FIG. 11G showed the uptake of the dendritic cell-targeting formulations according to embodiments of the present disclosure targeting BDMCs compared with conventional LNPs. FIG. 12A, FIG. 12B, FIG. 12C, FIG. 12D, FIG. 12E, FIG. 12F, and FIG. 12G showed the transfection of the targeting formulations BDMCs compared with LNPs without the compound of the present disclosure. The negative control was an LNP without using the present disclosure's novel targeting compound/formulation (i.e., “traditional LNP” as described herein). LNPs made using the compounds of the present disclosure at different molar ratios all showed higher uptake than the negative control (“traditional” LNP without using the compound of the present disclosure). The data also confirms that the LNPs of the present disclosure not only can deliver the payload into the targeted cells but also can transfect and allow the targeted cells to express the payload. Given the higher specificity towards the targeted cells, the transfection signals detected from the groups using the LNPs of the present disclosures were also significantly higher than those detected from the traditional group. This result suggests that using the LNPs of the present disclosure allows a lower dosage of the payload for a similar outcome.

Table showing the results of uptake and transfection (arbitrary unit of the FITC signals)

| | | FITC signal | FITC signal |
|---|---|---|---|
| | | intensity | intensity |
| | L5 | (A.U.) | (A.U.) |
| Experiment | the compound of the | derived from | derived from |
| 3-2 | present disclosure | uptake | transfection |
| Example 1 | 5 mol % Compound 22 | 12.0 | 1.58 |
| Example 2 | 10 mol % Compound 22 | 6.11 | 2.35 |
| Example 3 | 20 mol % Compound 22 | 11.0 | 1.88 |
| Example 4 | 5 mol % Compound 23 | 13.4 | 2.11 |
| Example 5 | 10 mol % Compound 23 | 13.6 | 1.67 |
| Example 6 | 20 mol % Compound 23 | 23.6 | 2.30 |
| Example 7 | None (traditional LNP) | 0.38 | 0.71 |
| (control) | | | |

### Experiment 3-3

To assess the binding of DC-SIGN to the LNPs of the present disclosure, ELISA plates were coated with exemplary LNPs in PBS at 4° C. overnight, respectively. The plates were incubated with diluted DC-SIGN ECD (15 to 0.075 nM in HEPES buffer containing 20 mM HEPES, 150 mM NaCl, 10 mM CaCl₂), 0.1% BSA) at pH 7.4, 6.0, and 5.0 for 1 hour at room temperature. The bound DC-SIGN ECD was detected using HRP-conjugated anti-DC-SIGN (B2) IgG antibody (Santa Cruz Biotechnology). After 1 hour of incubation at room temperature, the plates were treated with tetramethlybenzidine (TMB) for 10 min. The optical density was measured at 450 nm after adding 0.5 M sulfuric acid to the plates using a microplate reader. The apparent Kd was calculated using a nonlinear regression curve fit for total binding using GraphPad Prism.

Example B4: In Vivo Delivery of Luciferase mRNA-LNP

This experiment tested the targeted delivery of the LNPs of the present disclosure (shown in the table below) in vivo. The LNPs tested in this experiment carried mRNA encoding luciferase. Mice were injected intravenously with the LNPs (200 μL) and maintained for one hour or six hours before In vivo Imaging System (IVIS®) measurement. For the IVIS measurement, the animals were first anesthetized using the rodent anesthesia system with isoflurane (2.5% (vol/vol) in 0.2 L/min O2 flow). Then, the animals were injected intravenously with D-luciferin solution (dissolved in 1×PBS; 150 mg/kg body weight). After 3 minutes from the injection, the animals were scanned using the IVIS imaging system (data not shown). After imaging, the animals were euthanized in a CO₂ chamber. The organs (heart, lungs, liver, spleen, kidneys, and lymph nodes) of the animals were collected, and the luminescence was detected and quantified using the IVIS system.

Table of the exemplary LNPs tested in this experiment.

| | | | | | | L5 |
|---|---|---|---|---|---|---|
| | L1: | L2: | | | | the compound |
| | Ionizable | Phosphatidyl- | | L3: | L4: | of the present |
| Example | lipid | choline | | Cholesterol | PEG-Lipid | disclosure |
| 1 | 45 mol % | 9 | mol % | 34.5 mol % | 1.5 mol % | 10 mol % |
| | | | | | | Compound |
| | | | | | | 22 |
| 2 | 45 mol % | 9 | mol % | 34.5 mol % | 1.5 mol % | 10 mol % |
| | | | | | | Compound |
| | | | | | | 12 |
| 7 | 50 mol % | 10 | mol % | 38.5 mol % | 1.5 mol % | none |

(control)

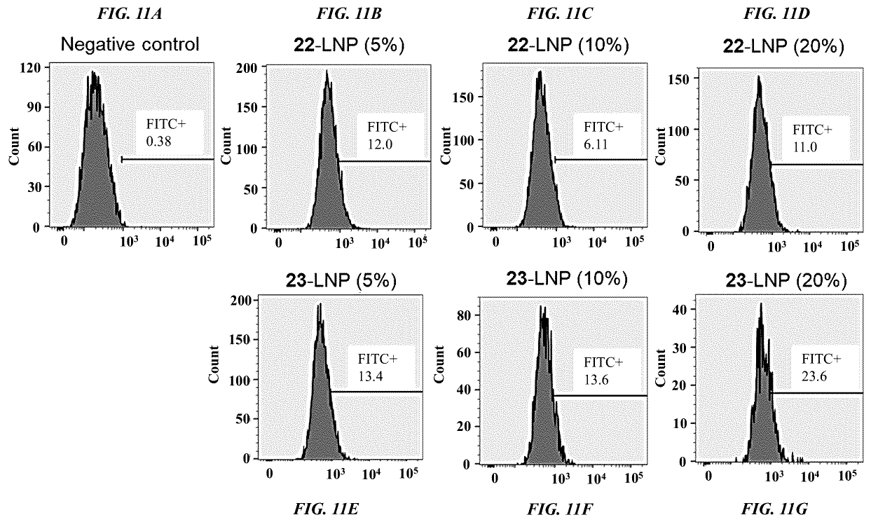

Results. The results (FIG. 13A, FIG. 13B, and FIG. 13C) show that both compound 22-LNP and compound 12-LNP tend to accumulate in spleens and lymph nodes. While compound 12-LNP also accumulated in livers, compound 22-LNP showed high-level specificities targeting spleens and lymph nodes. The results demonstrate the targeting delivery functionalities of the lipid nanoparticle formulations of the present disclosure, which matches the observations of the experiments above.

### Example B5: Humoral Immune Response Induced by LNPs

This experiment verified the capabilities of the LNPs of the present disclosure in delivering immunogenic cargos and inducing humoral immune responses in vivo. First, traditional LNPs (i.e., without using the compound of the present disclosure) and the LNPs using compound 24 of the present disclosure (see Sample 1 of Experiment 3-1) were prepared and carried COVID spike protein-encoding mRNA. A micelle type mRNA nanoparticle made from compound 24 and carrying the spike protein-encoding mRNA was also prepared for this experiment. Balb/c mice were separated into groups, and each group was intravenously injected with the traditional LNPs, LNP-compound 24, and compound 24-micelles, respectively, or injected with PBS as a negative control. Then, blood samples were collected from the experimental mice at 2 hours, 24 hours, and 48 hours after injections. The sera of the blood samples were obtained using centrifugation (3000×g, 10 minutes).

Cytokine concentration in the obtained sera was then determined using BD OptEIA™ Mouse ELISA Set. Briefly, 96-well plates were coated with anti-interleukin-4 (IL-4) antibody solution or anti-interferon-γ (IFNγ) antibody solution (1 μg/ml, 100 μl/well) and incubated at 4° C. overnight. Then, the plates were washed with PBST buffer (0.05% Tween 20 in PBS) and blocked using diluent buffer (10% FBS/PBS) at room temperature for 1 hour, followed by another washing procedure. The plates were then added with biotinylated detection antibodies and SA-HRP (100 μl/well) and incubated at room temperature for 1 hour. After that, the plates were washed with PBST buffer, and a substrate solution (100 μl/well) was added. The plates were then incubated at room temperature for 30 minutes in the dark. After stopping the development by adding a stop solution (50 μl/well), the plates were observed and signals were detected using an ELISA reader at 450 nm.

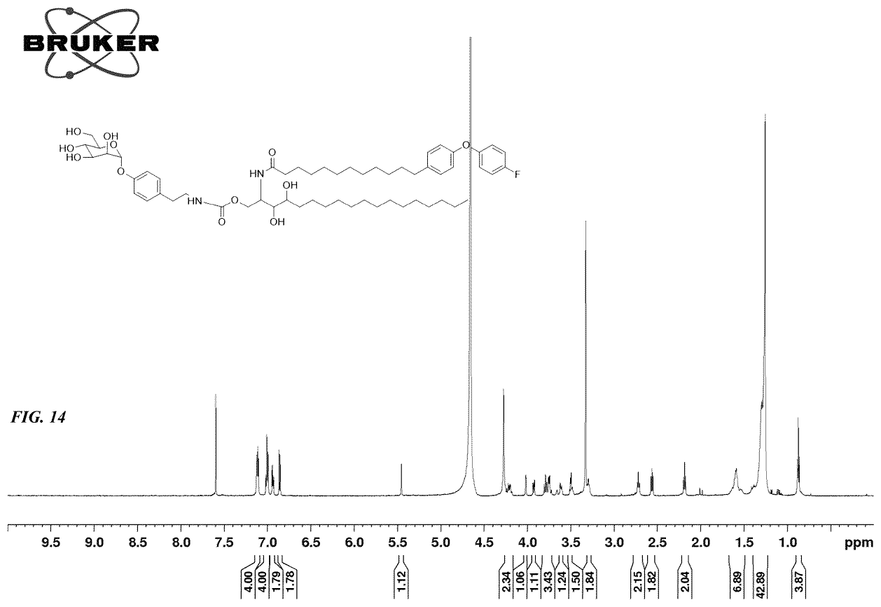

Result. The detection results are shown in FIG. 16A and FIG. 16B. The sera obtained from mice administered with the LNPs of the present disclosure contained detectably increased IFNγ and IL-4 after 2 hours of administration. This observation suggested that the LNPs of the present disclosure were able to deliver payload and induce humoral immune responses rapidly. In contrast, the traditional LNPs did not induce detectable humoral immune responses, showing that the targeted delivery capability of the present disclosure's LNPs was able to improve the efficiency of payload delivery thereby improving the desired effects.

### Example B6: Immunization

Animals. Balb/c mice (8 weeks) were purchased from the National Laboratory Animal Center, Taiwan. All the mice were maintained in a specific pathogen-free environment. Eight-week-old Balb/c mice were immunized i.m. twice at 2-week intervals. Each vaccination contains PBS (100 μl). Sera collected from immunized mice were subjected to ELISA analysis 10 days after the last immunization. The experimental protocol was approved by Academia Sinica's Institutional Animal Care and Utilization Committee (approval no. 22-08-1901).

LNPs. For neutralization assay, LNPs, according to an embodiment of the present disclosure, were prepared for this experiment. Two control LNPs were also prepared to compare the performance of the present disclosure's LNPs. The first control LNP was formed using SM-102 and DSPC (“L1+L2”) without using the compound of the present disclosure. The second control LNP was a Moderna product for Spikevax (“LNP (M)”). All tested LNPs carried mRNA cargo encoding SARC-CoV-2 spike protein. For IgG titer assay, LNPs of the present disclosure were prepared to carry either a mRNA encoding wild-type SARC-CoV-2 spike protein or a mRNA encoding wild-type SARC-CoV-2 spike protein with low-sugar modification.

Animal Immunizations. BALB/c mice aged 6 to 8 wk old (n=5) were immunized intramuscularly with 15 μg of LNPs in phosphate-buffered saline (PBS). Animals were immunized at week 0 and boosted with a second vaccination at week 2, and serum samples were collected from each mouse 2 weeks after the second immunization.

Pseudovirus neutralization assay. Pseudovirus was constructed by the RNAi Core Facility at Academia Sinica using a procedure similar to that described previously. Briefly, the pseudotyped lentivirus carrying SARS-COV-2 spike protein was generated by transiently transfecting HEK-293T cells with pCMV-AR8.91, pLAS2w.Fluc.Ppuro. HEK-293T cells were seeded one day before transfection, and indicated plasmids were delivered into cells using TransITR-LT1 transfection reagent (Mirus). The culture medium was refreshed at 16 hours and harvested at 48 hours and 72 hours post-transfection. Cell debris was removed by centrifugation at 4,000×g for 10 min, and the supernatant was passed through a 0.45-μm syringe filter (Pall Corporation). The pseudotyped lentivirus was aliquot and then stored at −80° C. To estimate the lentiviral titer by AlarmaBlue assay (Thermo Scientific), The transduction unit (TU) of SARS-CoV-2 pseudotyped lentivirus was estimated by using cell viability assay in responded to the limited dilution of lentivirus. In brief, HEK-293T cells stably expressing the human ACE2 gene were plated on a 96-well plate one day before lentivirus transduction. For the tittering pseudotyped lentivirus, different amounts of lentivirus were added into the culture medium containing polybrene (final concentration 8 μg/ml). Spin infection was carried out at 1,100×g in a 96-well plate for 30 minutes at 37° C. After incubating cells at 37° C. for 16 hr, the culture medium containing virus and polybrene was removed and replaced with fresh complete DMEM containing 2.5 μg/ml puromycin. After treating puromycin for 48 hrs, the culture media was removed, and the cell viability was detected using 10% AlamarBlue reagents according to the manufacturer's instructions. The survival rate of uninfected cells (without puromycin treatment) was set as 100%. The virus titer (transduction units) was determined by plotting the survival cells versus the diluted viral dose. For neutralization assay, heat-inactivated sera or antibodies were serially diluted and incubated with 1,000 TU of SARS-COV-2 pseudotyped lentivirus in DMEM for 1 h at 37° C. The mixture was then inoculated with 10,000 HEK-293T cells stably expressing the human ACE2 gene in a 96-well plate. The culture medium was replaced with fresh complete DMEM (supplemented with 10% FBS and 100 U/mL penicillin/streptomycin) at 16 h postinfection and continuously cultured for another 48 h. The expression level of the luciferase gene was determined by using the Bright-Glo Luciferase Assay System (Promega). The relative light unit (RLU) was detected by Tecan i-control (Infinite 500). The percentage of inhibition was calculated as the ratio of RLU reduction in the presence of diluted serum to the RLU value of no serum control using the formula (RLU^{control}−RLU_{Serum})/RLU control.

Measurement of serum IgG titer. ELISA was used to determine the IgG titer of the mouse serum. The wells of a 96-well ELISA plate (Greiner Bio-One) were coated with 100 ng SARS-COV-2 spike protein (ACROBiosystems, wild-type, Delta, or Omicron, respectively) in 100 mM sodium bicarbonate pH 8.8 at 4° C. overnight. The wells were blocked with 200 μl 5% skim milk in 1×PBS at 37° C. for 1 hour and washed with 200 μl PBST (1×PBS, 0.05% Tween 20, pH 7.4) three times. Mice serum samples with 2-fold serial dilution were added into wells for incubation at 37° C. for 2 hours and washed with 200 μl PBST six times. The wells were incubated with 100 μl HRP conjugated anti-mouse secondary antibody (1:10000, in PBS) at 37° C. for 1 hour and washed with 200 μl PBST six times. 100 μl horseradish peroxidase substrate (1-Step™ Ultra TMB-ELISA Substrate Solution) (Thermo Scientific™) was added into wells, followed by 100 μl 1M H₂SO₄. After incubation for 30 minutes, absorbance (OD 450 nm) was measured using SpectraMax M5.

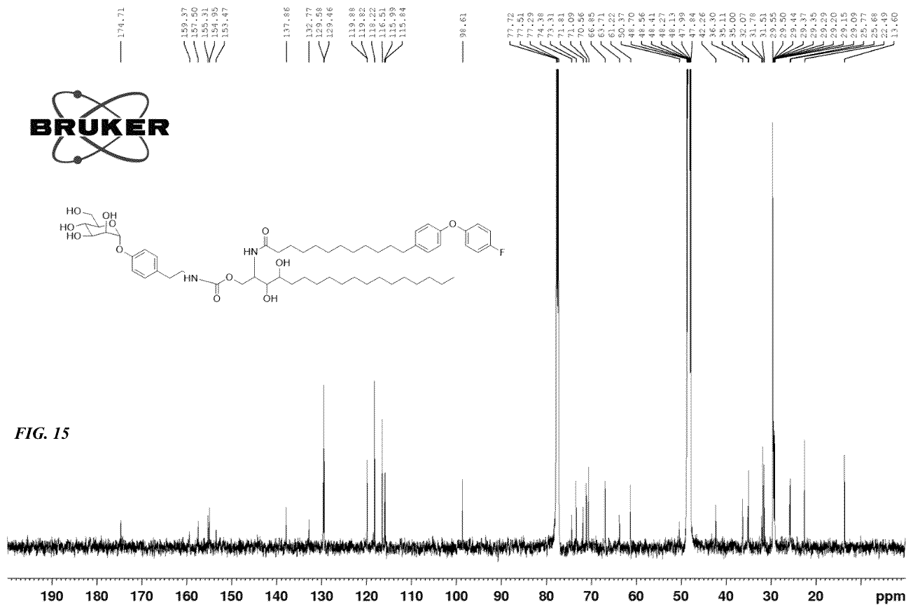

Results. FIG. 17 shows that all tested LNPs carrying the mRNA cargo were able to deliver and express the mRNA in vivo, thereby invoking immune responses that resulted in neutralization inhibition. Nevertheless, the inhibitory effect of those tested LNPs differed as the dilution factor increased. Both L1+L2 LNP and LNP (M) only showed a slightly higher inhibitory effect at 1:5000 dilution compared with the negative control, but the LNP of the present disclosure maintained around 40% inhibitory effect. The data demonstrates that the LNP of the present disclosure was able to invoke immune responses at a much lower concentration than other LNPs tested in this experiment.

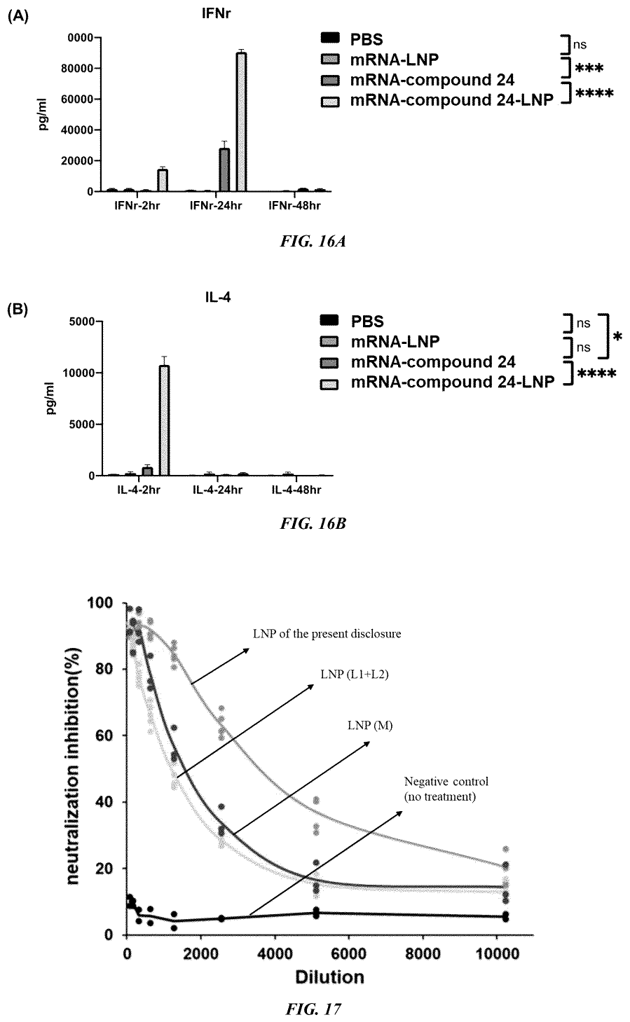

FIG. 18 verifies that the LNPs of the present disclosure were capable of inducing antigen-specific IgG in vivo. The LNP, carrying mRNA encoding wide-type spike protein, induced IgGs that were still able to recognize the spike proteins of both the Delta variant and Omicron variant at a good level. The data shows that the targeted delivery feature of the LNP can at least partially overcome the immune escape due to the spike protein variations between variants.

Furthermore, it was observed that LNP carrying wild-type SARS-COV-2 spike protein-encoding mRNA (“WT LNP”) and LNP carrying mRNA encoding a low-sugar modified spike protein (“low-sugar LNP”) induced comparable IgG titers against wide-type viruses, the WT LNP had lower IgG titers against the Delta and Omicron strains, suggesting an immune escape. In contrast, the low-sugar LNP maintains a high level of IgG titers against the two variant strains. The results demonstrate that removing glycan shields improves the immunogenicity of the LNP formulations.

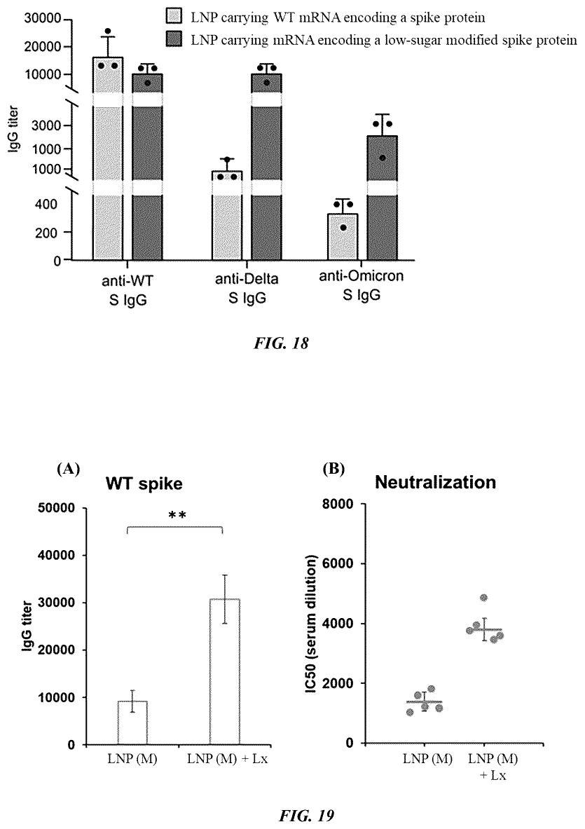

FIG. 19 shows the results of an additional experiment. In this experiment, a Moderna LNP was prepared using Moderna's proprietary formulation. In addition, LNP made using the Moderna formulation and adding a compound of the present disclosure was also prepared to test whether the compound of the present disclosure improves the performance of the Moderna formulation. Animals were administered with the LNPs, and IgG titer assay and neutralization assay were all performed as described above in this example. The results demonstrate that the compound of the present disclosure increased the spike protein-specific IgGs. The serum obtained from mice administered with the LNP of the present disclosure's compound also exhibited better neutralization. This experiment confirmed the targeted delivery feature of the compound of the present disclosure and verified that it can be applied to commercially available LNP formulations.

### EMBODIMENTS

Embodiment 1. A nucleic acid, configured to encode a recombinant spike protein, wherein the recombinant spike protein has an N-linked glycosylation site in an S1 domain or an S2 domain thereof, provided that a stem region thereof is devoid of an N-linked glycosylation site.

Embodiment 2. The nucleic acid of Embodiment 1, wherein both the S1 domain and the S2 domain of the recombinant spike protein comprise an N-linked glycosylation site.

Embodiment 3. The nucleic acid of Embodiment 2, wherein the recombinant spike protein comprises an N-linked glycosylation site in a receptor binding domain (RBD) thereof.

Embodiment 4. The nucleic acid of any one of Embodiments 1 to 3, wherein the stem region comprises an amino acid substitution of asparagine (N) at a N-linked glycosylation sequon (N-Xa-S/T), wherein N denotes an asparagine (N) residue, S denotes a serine(S) residue, T denotes a threonine (T) residue, and Xa in the sequon is any amino acid residue except proline.

Embodiment 5. The nucleic acid of Embodiment 4, wherein the N residue is substituted to a glutamine (Q) residue.

Embodiment 6. The nucleic acid of any one of Embodiments 1 to 5, wherein the stem region of the recombinant spike protein comprises an amino acid sequence at least 80%, 85%, 90%, 91%, 92%, 93%, 94%, 95%, 96%, 97%, 98%, or 99%, or 100% identical to SEQ ID NO: 07:

LHVTYVPA QEKXFTTAPA ICHDGKAHFP REGVFVSXGT
HWFVTQRXFY EPQIITTDNT FVSGNCDVVI GIVXNTVYDP
LQPELDSFKE ELDKYFKXHT SPDVDLGDIS GIXASVVNIQ
KEIDRLNEVA KNLNESLIDL QELGKYEQYI K,

wherein X denotes any amino acid, provided that each one of the X12, X36, X46, X72, X96, and X111 is not an Asn (N) residue.

Embodiment 7. The nucleic acid of Embodiment 6, wherein at least one of the X12, X36, X46, X72, X96, and X111 is an Gln (Q) residue.

Embodiment 8. The nucleic acid of Embodiment 6 or Embodiment 7, wherein the stem region of the recombinant spike protein comprises an amino acid sequence at least 80%, 85%, 90%, 91%, 92%, 93%, 94%, 95%, 96%, 97%, 98%, or 99%, or 100% identical to SEQ ID NO: 08, provided that each one of the Q12, Q36, Q46, Q72, Q96, and Q111 is not Asn (N) residue.

Embodiment 9. The nucleic acid of any one of Embodiments 1 to 8, wherein the recombinant spike protein comprises a S2 domain comprising an amino acid sequence at least 80%, 85%, 90%, 91%, 92%, 93%, 94%, 95%, 96%, 97%, 98%, or 99%, or 100% identical to SEQ ID NO: 05, wherein X denotes any amino acid, provided that each one of the X388, X412, X422, X448, X472, and X487 is not an Asn (N) residue.

Embodiment 10. The nucleic acid of Embodiment 9, wherein at least one of the X23, X31, and X115 is an Asn (N) residue.

Embodiment 11. The nucleic acid of Embodiment 9 or Embodiment 10, wherein at least one of the X388, X412, X422, X448, X472, and X487 is an Gln (Q) residue.

Embodiment 12. The nucleic acid of any one of Embodiments 9 to 11, wherein the stem region of the recombinant spike protein comprises an amino acid sequence at least 80%, 85%, 90%, 91%, 92%, 93%, 94%, 95%, 96%, 97%, 98%, or 99%, or 100% identical to t SEQ ID NO: 06, provided that each one of the Q388, Q412, Q422, Q448, Q472, and Q487 not Asn (N) residue.

Embodiment 13. The nucleic acid of any one of Embodiments 1 to 12, wherein the recombinant spike protein comprises an amino acid sequence at least 80%, 85%, 90%, 91%, 92%, 93%, 94%, 95%, 96%, 97%, 98%, or 99%, or 100% identical to SEQ ID NO: 02, wherein X denotes any amino acid, provided that at least one of X17, X61, X74, X122, X149, X165, X234, X282, X331, X343, X394, X487, X603, X616, X657, X709, X717, and X801 is an Asn (N) residue, and each one of the X1074, X1098, X1108, X1134, X1158, and X1173 is not an Asn (N) residue.

Embodiment 14. The nucleic acid of Embodiment 13, wherein the recombinant spike protein comprises an amino acid sequence at least 80%, 85%, 90%, 91%, 92%, 93%, 94%, 95%, 96%, 97%, 98%, or 99%, or 100% identical to SEQ ID NO: 04, provided that each one of the Q1074, Q1098, Q1108, Q1134, Q1158, and Q1173 is not an Asn (N) residue.

Embodiment 15. The nucleic acid of any one of Embodiments 1 to 14, comprising a nucleotide sequence at least 80%, 85%, 90%, 91%, 92%, 93%, 94%, 95%, 96%, 97%, 98%, or 99%, or 100% identical to SEQ ID NO: 11.

Embodiment 16. The nucleic acid of any one of Embodiments 1 to 5, wherein the stem region of the recombinant spike protein comprises an amino acid sequence at least 80%, 85%, 90%, 91%, 92%, 93%, 94%, 95%, 96%, 97%, 98%, or 99%, or 100% identical to SEQ ID NO: 18, wherein X denotes any amino acid, provided that each one of the X12, X36, X46, X72, X96, and X111 is not an Asn (N) residue.

Embodiment 17. The nucleic acid of Embodiment 16, wherein the stem region of the recombinant spike protein comprises an amino acid sequence at least 80%, 85%, 90%, 91%, 92%, 93%, 94%, 95%, 96%, 97%, 98%, or 99%, or 100% identical to SEQ ID NO: 18, wherein at least one of X12, X36, X46, X72, X96, and X111 is an Gln (Q) residue.

Embodiment 18. The nucleic acid of Embodiment 16 or Embodiment 17, wherein the stem region of the recombinant spike protein comprises an amino acid sequence at least 80%, 85%, 90%, 91%, 92%, 93%, 94%, 95%, 96%, 97%, 98%, or 99%, or 100% identical to SEQ ID NO: 19, provided that each one of the Q12, Q36, Q46, Q72, Q96, and Q111 is not Asn (N) residue.

Embodiment 19. The nucleic acid of any one of Embodiments 1 to 5 and Embodiments 16 to 18, wherein the recombinant spike protein comprises a S2 domain comprising an amino acid sequence at least 80%, 85%, 90%, 91%, 92%, 93%, 94%, 95%, 96%, 97%, 98%, or 99%, or 100% identical to SEQ ID NO: 16, wherein X denotes any amino acid, provided that each one of the X23, X31, X115, X388, X412, X422, X448, X472, and X487 is not an Asn (N) residue.

Embodiment 20. The nucleic acid of any one of Embodiments 1 to 5 and Embodiments 16 to 18, wherein the recombinant spike protein comprises a S2 domain comprising an amino acid sequence at least 80%, 85%, 90%, 91%, 92%, 93%, 94%, 95%, 96%, 97%, 98%, or 99%, or 100% identical to SEQ ID NO: 16, wherein X denotes any amino acid, provided that each one of the X388, X412, X422, X448, X472, and X487 is not an Asn (N) residue.

Embodiment 21. The nucleic acid of Embodiment 20, wherein at least one of the X23, X31, and X115 is an Asn (N) residue.

Embodiment 22. The nucleic acid of Embodiment 20 or Embodiment 21, wherein at least one of the X388, X412, X422, X448, X472, and X487 is an Gln (Q) residue.

Embodiment 23. The nucleic acid of any one of Embodiments 20 to 22, wherein the stem region of the recombinant spike protein comprises an amino acid sequence at least 80%, 85%, 90%, 91%, 92%, 93%, 94%, 95%, 96%, 97%, 98%, or 99%, or 100% identical to SEQ ID NO: 17, provided that each one of the Q388, Q412, Q422, Q448, Q472, and Q487 is not an Asn (N) residue.

Embodiment 24. The nucleic acid of any one of Embodiments 1 to 5 and Embodiments 16 to 23, wherein the recombinant spike protein comprises an amino acid sequence at least 80%, 85%, 90%, 91%, 92%, 93%, 94%, 95%, 96%, 97%, 98%, or 99%, or 100% identical to SEQ ID NO: 13, wherein X denotes any amino acid, provided that at least one of the X61, X74, X122, X149, X163, X232, X280, X329, X341, X392, X485, X601, X614, X655, X707, X715, and X799 is an Asn (N) residue, and each one of the X1072, X1096, X1106, X1132, X1156, and X1171 is not an Asn (N) residue.

Embodiment 25. The nucleic acid of Embodiment 24, wherein the recombinant spike protein comprises an amino acid sequence at least 80%, 85%, 90%, 91%, 92%, 93%, 94%, 95%, 96%, 97%, 98%, or 99%, or 100% identical to SEQ ID NO: 15, provided that each one of Q1072, Q1096, Q1106, Q1132, Q1156, and Q1171 is not Asn (N) residue.

Embodiment 26. The nucleic acid of any one of Embodiments 1 to 5 and 16 to 25, comprising a nucleotide sequence at least 80%, 85%, 90%, 91%, 92%, 93%, 94%, 95%, 96%, 97%, 98%, or 99%, or 100% identical to SEQ ID NO: 22.

Embodiment 27. The nucleic acid of any one of Embodiments 1 to 26, wherein the nucleic acid is deoxyribonucleic acid (DNA) or ribonucleic acid (RNA).

Embodiment 28. The nucleic acid of any one of Embodiments 1 to 27, wherein the nucleic acid is a messenger RNA (mRNA).

Embodiment 29. An expression vector, comprising the nucleic acid of any one of Embodiments 1 to 28.

Embodiment 30. The expression vector of Embodiment 29, wherein the nucleic acid further comprises a promoter, a 5′ untranslated region (5′UTR), a 3′ untranslated region (3′UTR), a 5′ cap, a poly-A tail, or a combination thereof.

Embodiment 31. The expression vector of Embodiment 29 or Embodiment 30, wherein the expression vector is a lipid nanoparticle, a liposome, a polymersome, a viral particle, a plasmid, or a bead.

Embodiment 32. The expression vector of Embodiment 31, wherein the expression vector is a lipid nanoparticle, and the lipid nanoparticle comprises a membrane defining an inner space, and wherein the membrane encompasses the nucleic acid, and the membrane is formed with a plurality of lipid components comprising a bi-functional compound, and the bi-functional compound comprises:

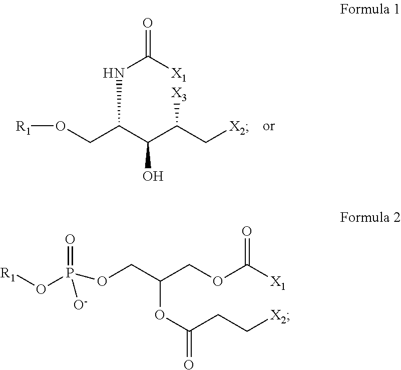

wherein R¹ comprises a substituted or non-substituted glycosyl group; wherein X₁ and X₂ are each independently hydrogen, C₁₋₃₀ alkyl, C₁₋₃₀ alkenyl, C₁₋₃₀ alkynyl, aryl, aryloxy, or a substituted version thereof, or —(CH₂)nX₄, n is 0 to 30, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof; and wherein X₃ is hydrogen, C₁₋₆ alkyl, or hydroxyl.

Embodiment 33. The expression vector of Embodiment 32, wherein R¹ comprises a formula of R₂—R_{A}—, wherein R_{A} is an attachment group and R₂ is the substituted or non-substituted glycosyl group, and wherein the attachment group comprises an aryl, an alkyl, an amide, an alkylamide, a substituted version thereof, a combination thereof, or a covalent bond.

Embodiment 34. The expression vector of Embodiment 33, wherein R_{A} comprises the aryl having 0 to 3 substituents, wherein the substituent is C₁₋₆ alkyl, halide, or C₁₋₆ alkyl halide.

Embodiment 35. The expression vector of Embodiment 34, wherein R_{A} further comprises a polyethylene glycol (PEG) moiety having 2 to 72 (OCH₂CH₂) subunits.

Embodiment 36. The expression vector of any one of Embodiments 32 to 35, wherein the glycosyl group comprises mannoside, fucoside, or a combination thereof.

Embodiment 37. The expression vector of any one of Embodiments 32 to 35, wherein the glycosyl group comprises a terminal mannoside, a terminal fucoside, or both.

Embodiment 38. The expression vector of any one of Embodiments 32 to 37, wherein the glycosyl group comprises a mono-mannoside, a di-mannoside, or a tri-mannoside.

Embodiment 39. The expression vector of Embodiment 38, wherein the tri-mannoside is a linear or branched tri-mannoside.

Embodiment 40. The expression vector of Embodiment 39, wherein the branched tri-mannoside is a α-1,3-α-1,6-trimannoside.

Embodiment 41. The expression vector of any one of Embodiments 32 to 40, wherein R¹ is a substituted glycosyl group.

Embodiment 42. The expression vector of Embodiment 41, wherein the glycosyl group comprises 1 to 6 substituents, wherein the substituent is C₁₋₆ alkyl, C₁₋₆ alkenyl, halogen, C₁₋₆ alkyl halide, C₁₋₆ alkoxy, amine, nitro, C₁₋₆ alkyl amine, amide, azido, aryl, cycloalkyl, heterocycloalkyl, sulfite, or a substituted version thereof, or a combination thereof.

Embodiment 43. The expression vector of Embodiment 42, wherein the substituent of the glycosyl group is selected from the group consisting of aryl, 5-membered cycloalkyl, 6-membered cycloalkyl, 5-membered heterocycloalkyl, and 6-membered heterocycloalkyl, and a substituted version thereof, which comprises 1 to 6 substituents selected from the group consisting of C₁₋₆ alkyl, halogen, C₁₋₆ alkyl halide, C₁₋₆ alkoxy, amine, nitro, C₁₋₆ alkyl amine, amide, azido, carboxyl, hydroxyl, aryl, cycloalkyl, heterocycloalkyl, or a substituted version thereof, or a combination thereof.

Embodiment 44. The expression vector of Embodiment 42 or Embodiment 43, wherein the substituent of the glycosyl group is a substituted or non-substituted aryl, optionally the substituent of the glycosyl group is a phenyl substituted with OH, CH₃, NH₂, CF₃, OCH₃, F, Br, Cl, NO₂, N₃, or a combination thereof.

Embodiment 45. The expression vector of Embodiment 42 or Embodiment 43, wherein the heterocycloalkyl comprises 1 to 3 heteroatoms, selected from the group consisting of O, S, and N.

Embodiment 46. The expression vector of any one of Embodiments 32 to 45, wherein R¹ is selected from the group consisting of:

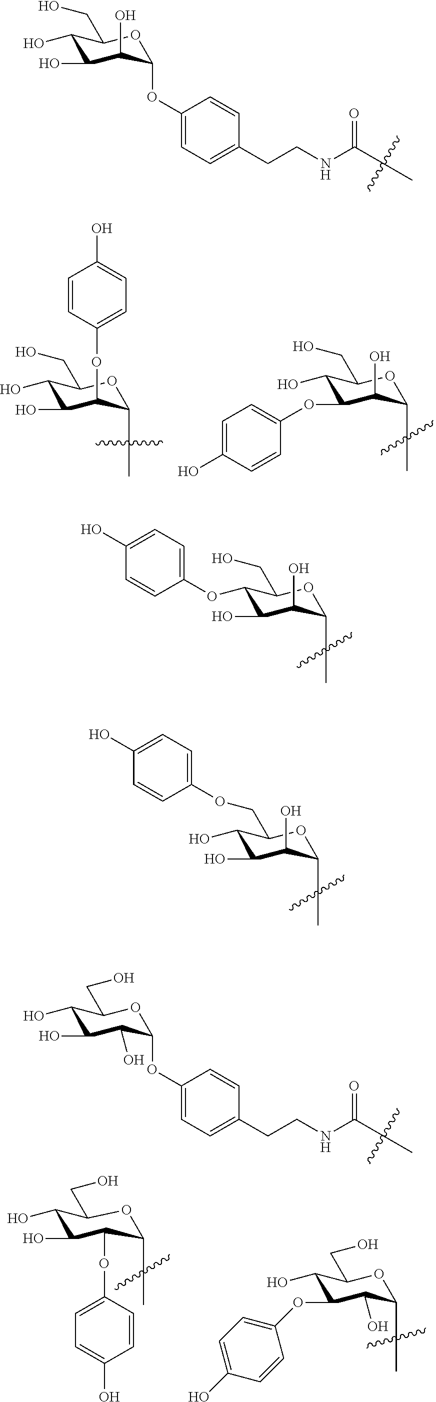

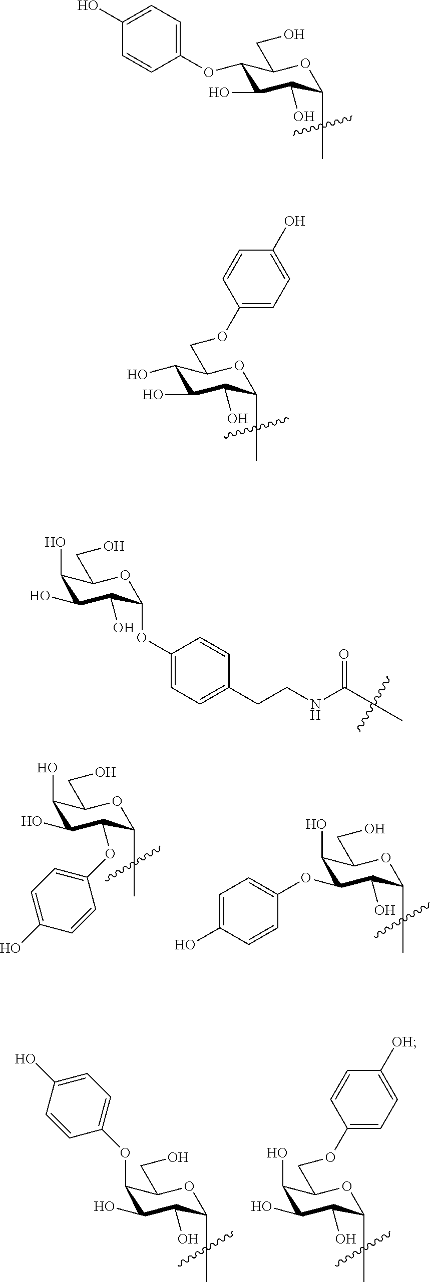

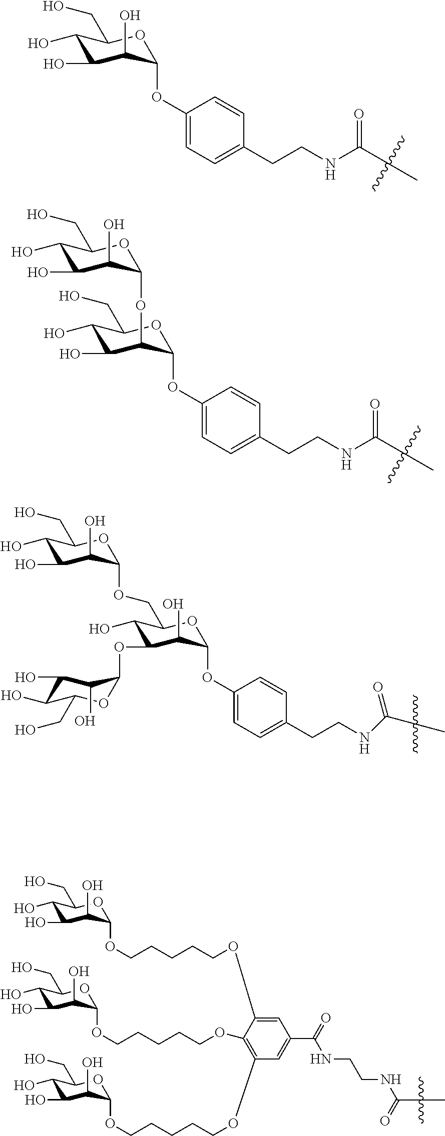

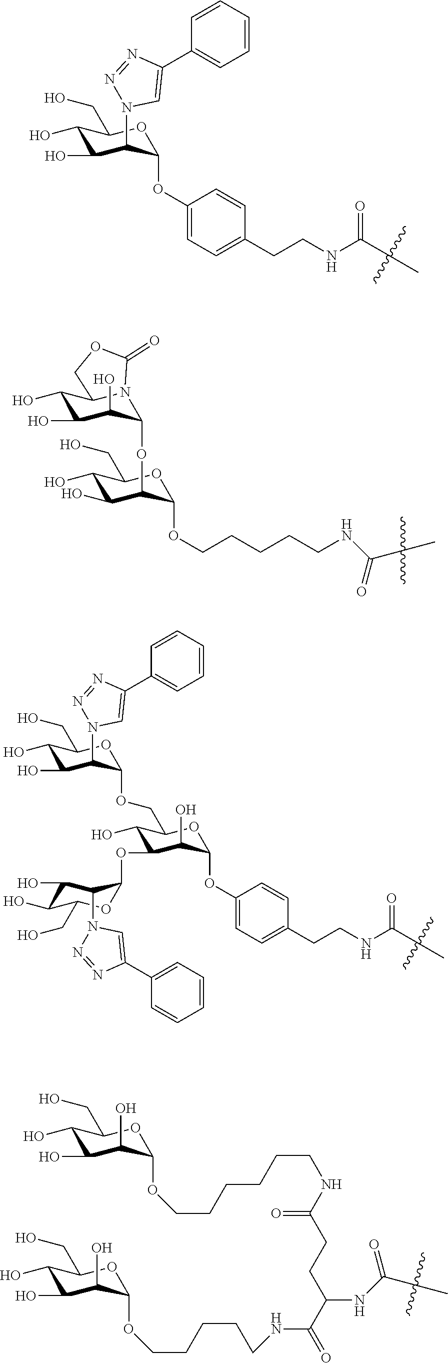

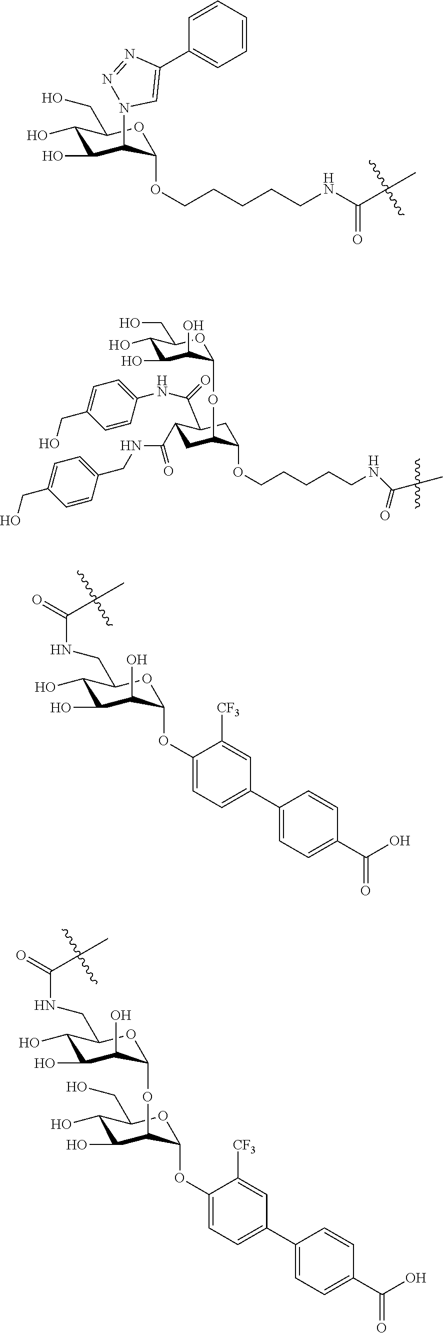

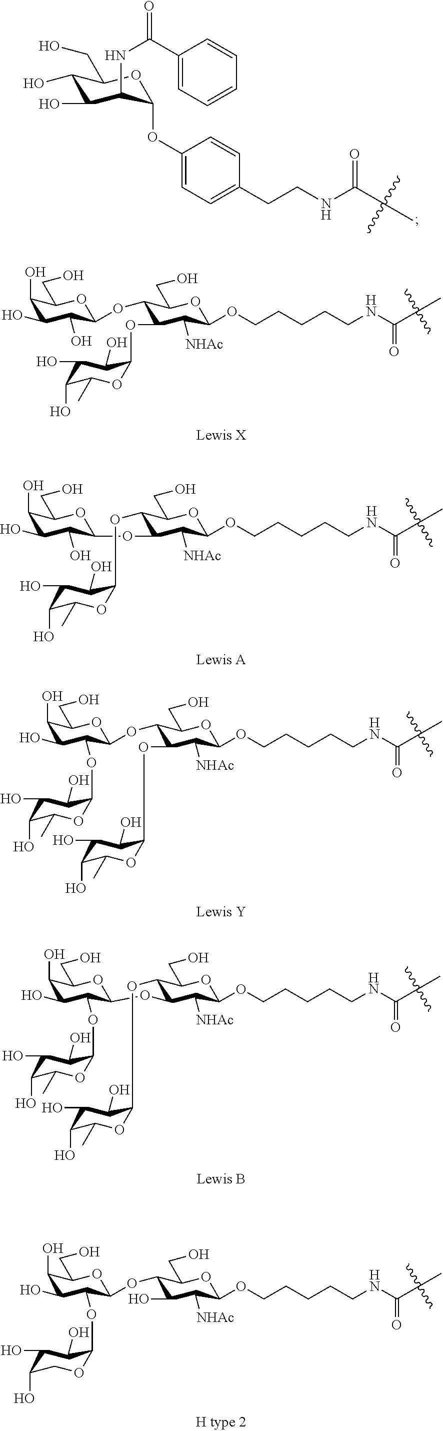

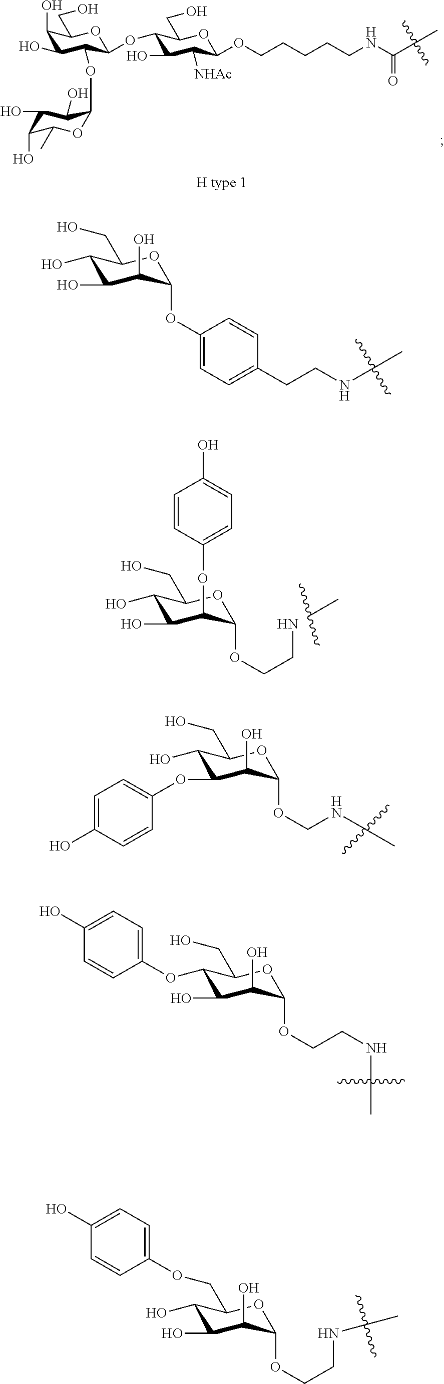

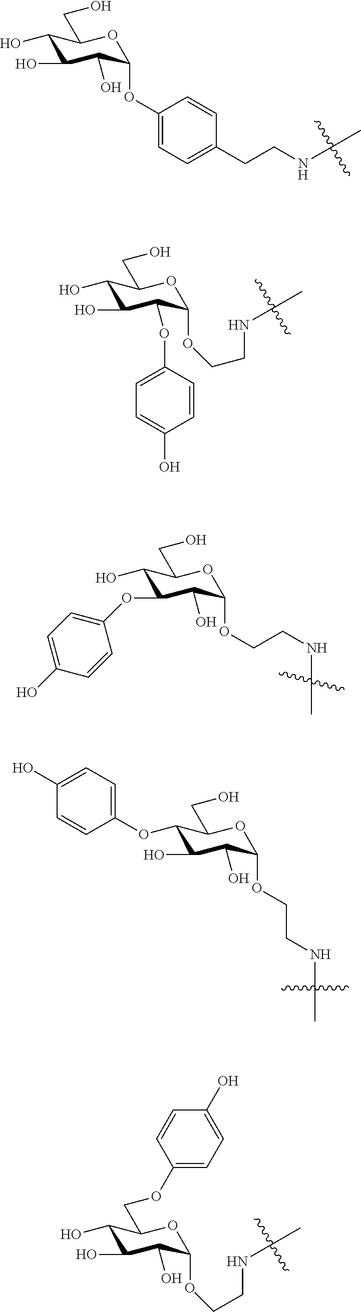

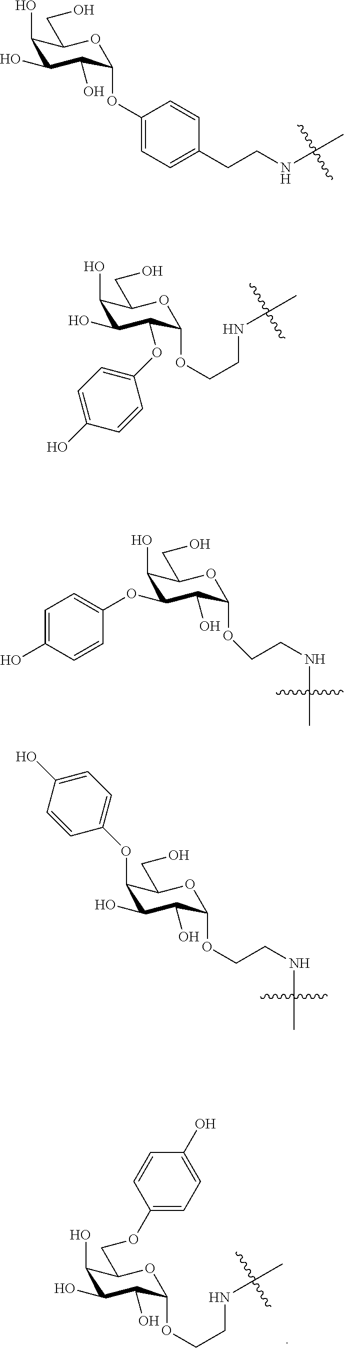

Embodiment 47. The expression vector of any one of Embodiments 32 to 46, wherein the compound is of Formula 1.

Embodiment 48. The expression vector of any one of Embodiments 32 to 47, wherein the compound is of Formula 2.

Embodiment 49. The expression vector of Embodiment 48, wherein the compound is of Formula 3:

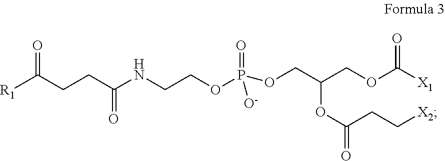

and
wherein R¹ is selected from the group consisting of:

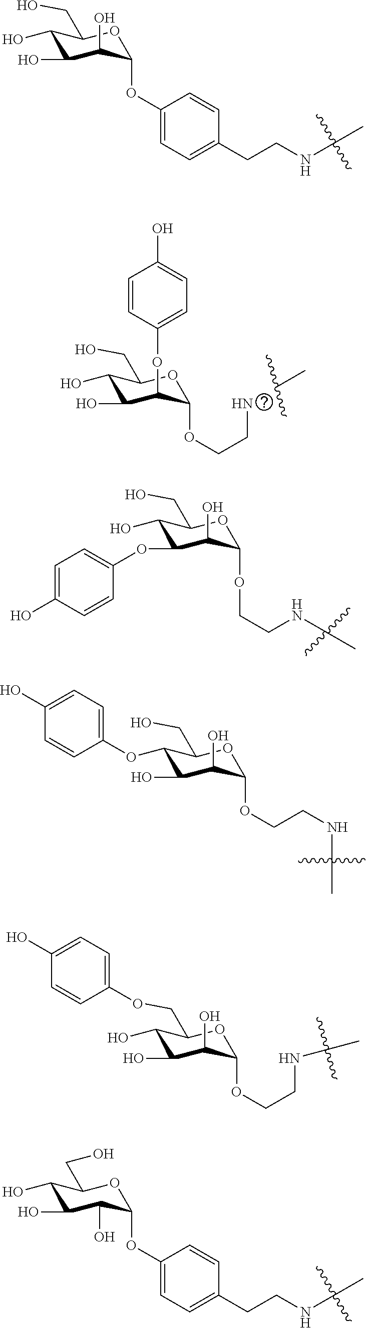

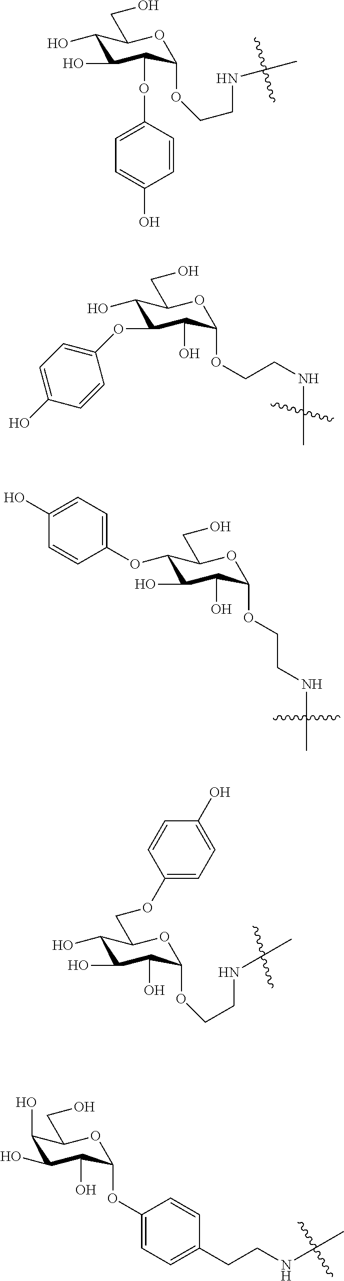

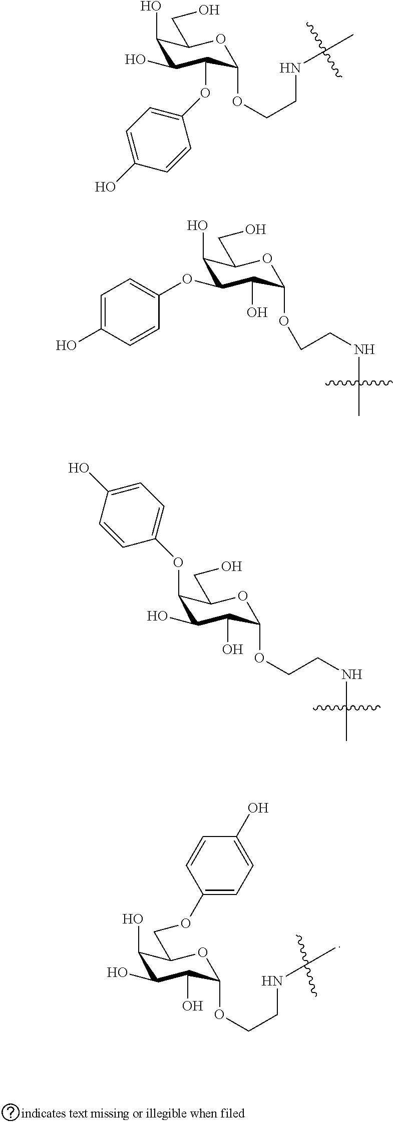

Embodiment 50. The expression vector of any one of Embodiments 32 to 49, wherein at least one of X₁ and X₂ comprises a saturated hydrocarbon chain, comprising at least 2, 3, 4, 5, 6, 7, 8, 9, 10, 11, 12, 13, 14, 15, 16, 17, 18, 19, 20, 22, 24, 26, 28, or 30 carbons.

Embodiment 51. The expression vector of any one of Embodiments 32 to 50, wherein X₁ and X₂ are each independently hydrogen, C₄₋₃₀ alkyl, C₄₋₃₀ alkenyl, C₄₋₃₀ alkynyl, aryl, aryloxy, or a substituted version thereof, or —(CH₂)nX₄, n is 4 to 30, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof.

Embodiment 52. The expression vector of Embodiment 51, wherein X1 and X2 are each independently hydrogen, C₈₋₃₀ alkyl, C₈₋₃₀ alkenyl, C₈₋₃₀ alkynyl, aryl, aryloxy, or a substituted version thereof, or —(CH₂)nX₄, n is 8 to 30, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof.

Embodiment 53. The expression vector of any one of Embodiments 32 to 52, provided that when one of X₁ and X₂ is hydrogen, the other one is not hydrogen.

Embodiment 54. The expression vector of any one of Embodiments 32 to 53, wherein X₄ is an aryl, aryloxy, heterocyclic group, cycloalkyl, heterocycloalkyl, or a combination thereof, and wherein X₄ comprises 0 to 6 substituents, selected from the group consisting of C₁₋₆ alkyl, halogen, C₁₋₆ alkyl halogen, and C₁₋₆ alkoxy.

Embodiment 55. The expression vector of Embodiment 54, wherein the substituent is CH₃, CF₃, F, or OCH₃.

Embodiment 56. The expression vector of Embodiment 54 or Embodiment 55, wherein X₄ comprises 1 to 3 substituents.

Embodiment 57. The expression vector of any one of Embodiments 54 to 56, wherein X₄ is —R₃—O—R₄, wherein R₃ and R₄ are each independently aryl, heterocyclic group, cycloalkyl, heterocycloalkyl, each comprising 0 to 6 substituents selected from the group consisting of C₁₋₆ alkyl, halogen, C₁₋₆ alkyl halogen, and C₁₋₆ alkoxy.

Embodiment 58. The expression vector of any one of Embodiments 32 to 57, wherein one of X₁ and X₂ is C₁₅₋₃₀ alkyl, and the other one is —(CH₂)nX₄.

Embodiment 59. The expression vector of any one of Embodiments 32 to 58, wherein X4 is selected from the group consisting of:

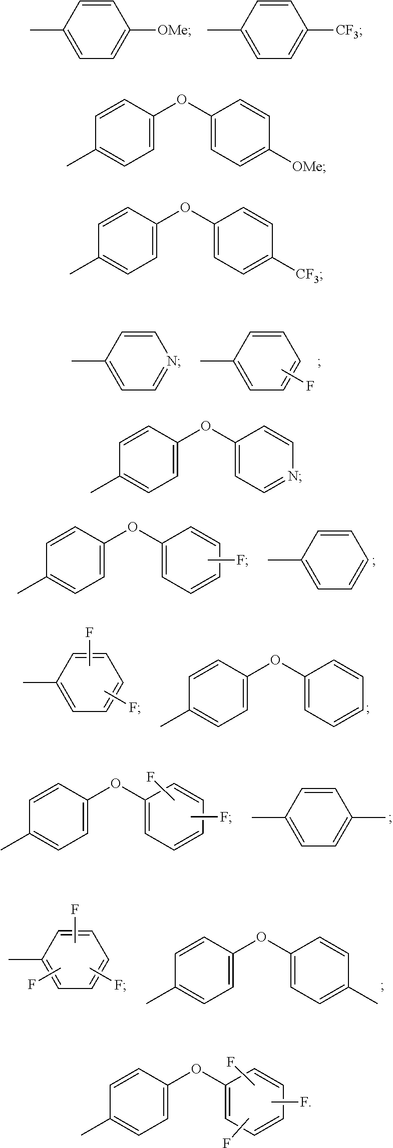

Embodiment 60. The expression vector of any one of Embodiments 32 to 59, wherein the compound is selected from the group consisting of:

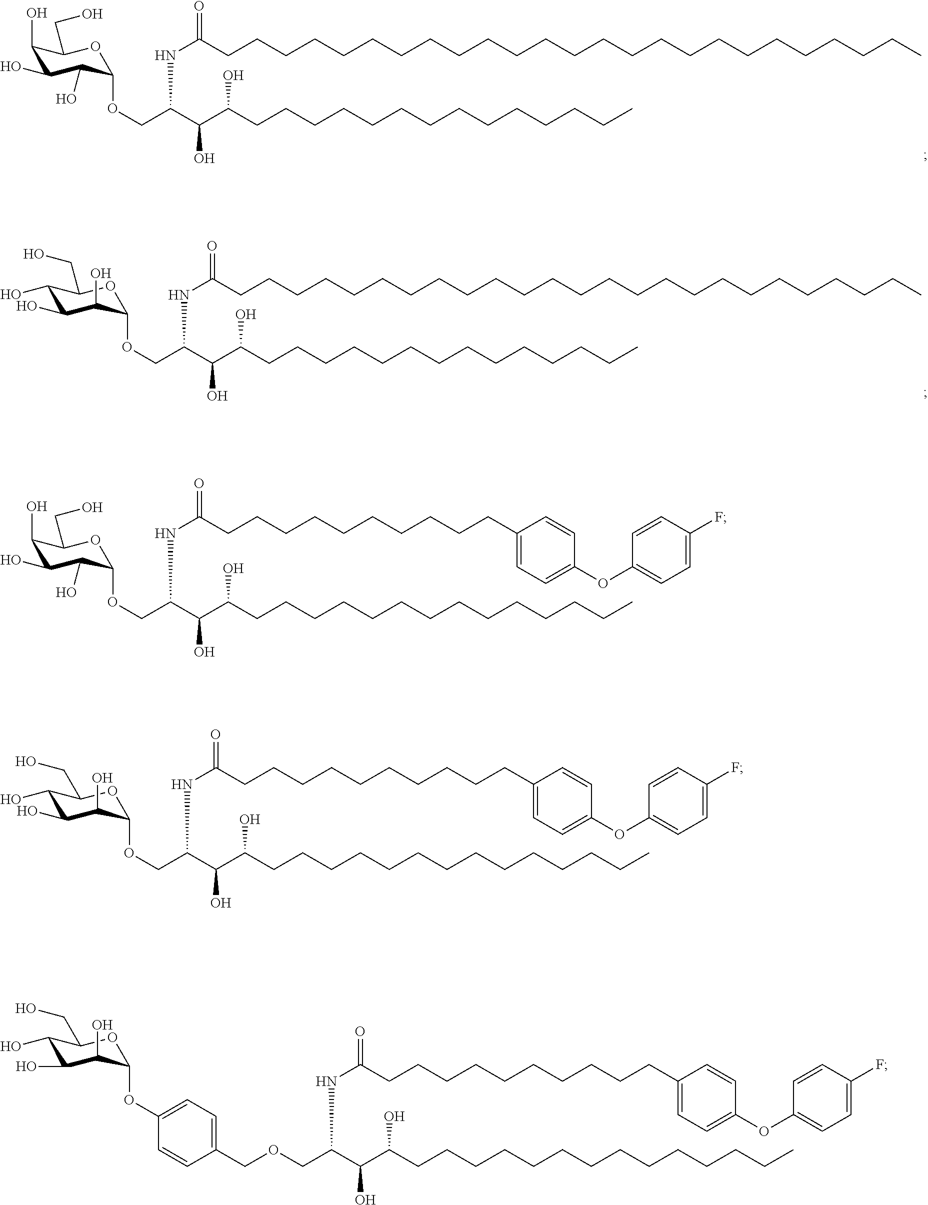

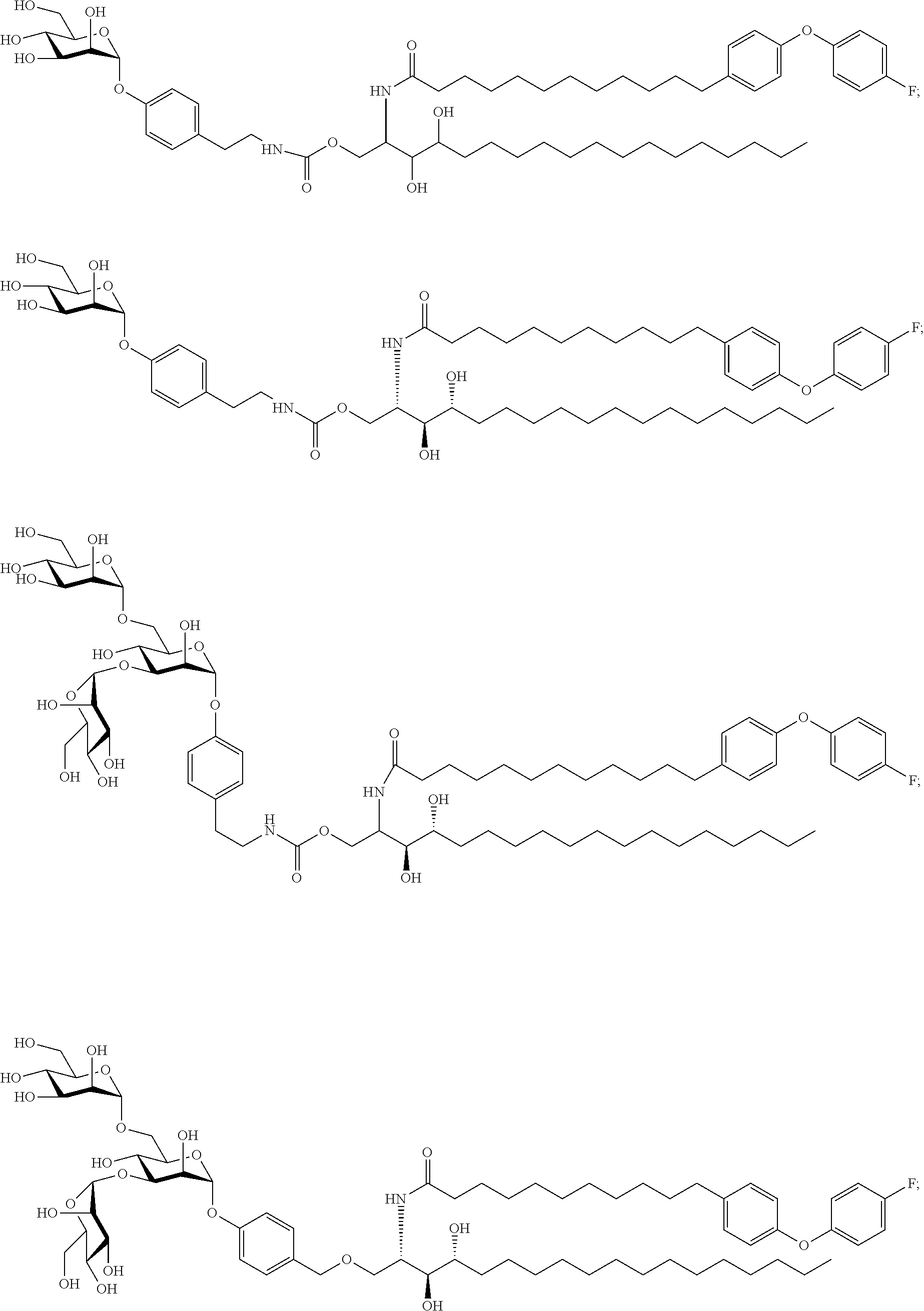

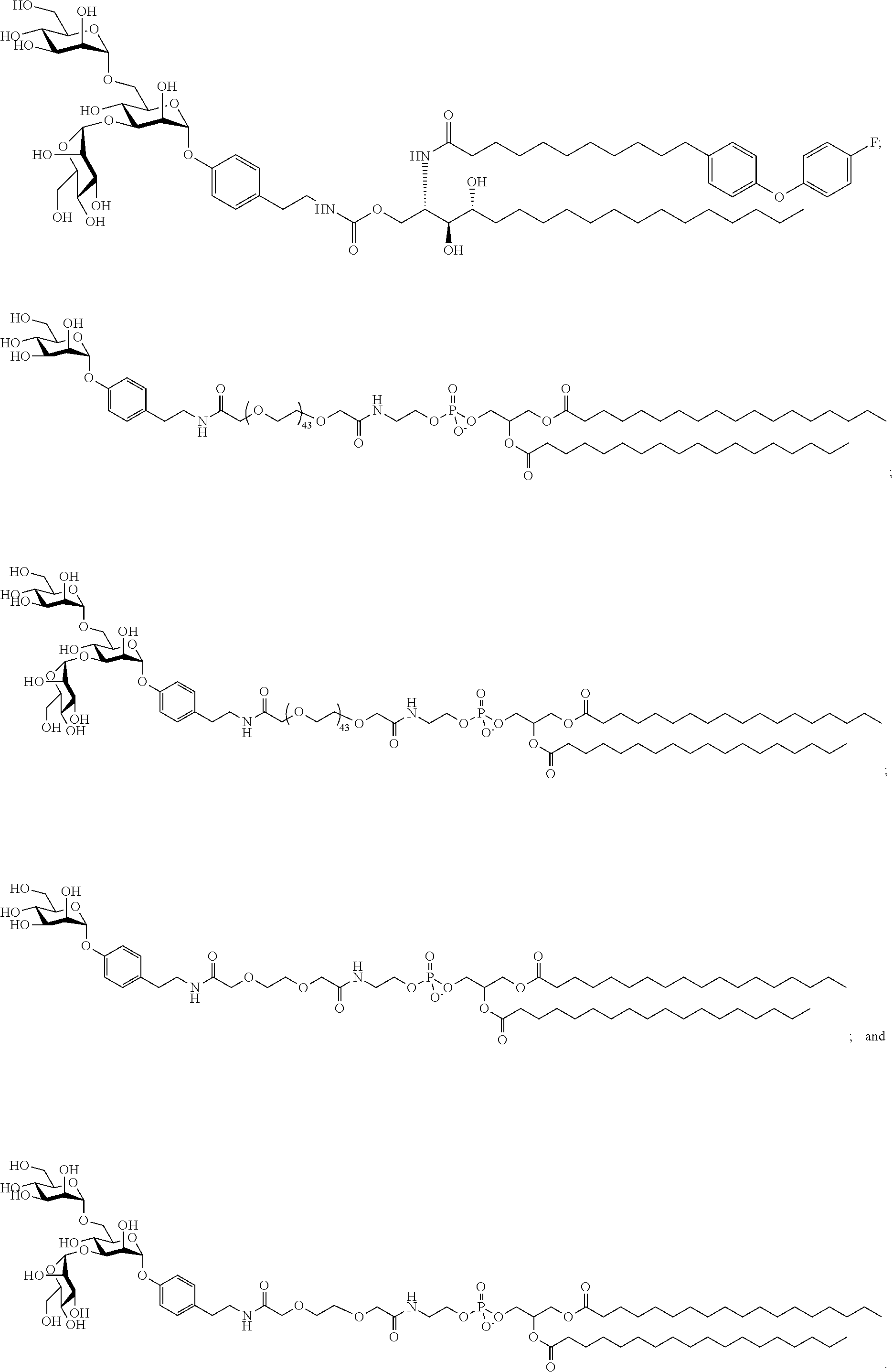

Embodiment 61. The expression vector of any one of Embodiments 32 to 60, wherein the component is not glycolipid C34 or α-galactosylceramide.

Embodiment 62. The expression vector of any one of Embodiments 32 to 61, wherein the plurality of the lipid components further comprises an ionizable lipid, a helper lipid, or a combination thereof.

Embodiment 63. The expression vector of Embodiment 62, wherein the ionizable lipid comprises heptadecan-9-yl 8-[2-hydroxyethyl-(6-oxo-6-undecoxyhexyl)amino]octanoate (SM-102™), (4-hydroxybutyl) azanediyl)bis(hexane-6,1-diyl)bis(2-hexyldecanoate) (ALC-0315™, Pfizer), or a combination thereof.

Embodiment 64. The expression vector of Embodiment 62 or Embodiment 63, wherein the helper lipid comprises a phosphatidylcholine, a cholesterol or a derivative thereof, a polyethylene glycol-lipid (PEG-lipid), or a mixture thereof.

Embodiment 65. The expression vector of Embodiment 64, wherein the phosphatidylcholine comprises distearoylphosphatidylcholine (DSPC), dioleoylphosphatidylethanolamine (DPOE), or a mixture thereof.

Embodiment 66. The expression vector of Embodiment 64 or Embodiment 65, wherein the cholesterol or a derivative thereof is a cholesterol, campesterol, beta-sitosterol, brassicasterol, ergosterol, dehydroergosterol, stigmasterol, fucosterol, DC-cholesterol HCl, OH-Chol, HAPC-Chol, MHAPC-Chol, DMHAPC-Chol, DMPAC-Chol, cholesteryl chloroformate, GL67, cholesteryl myristate, cholesteryl oleate, cholesteryl nervonate, LC10, cholesteryl hemisuccinate, (3β,5β)-3-hydroxycholan-24-oic acid, alkyne cholesterol, 27-alkyne cholesterol, E-cholesterol alkyne, trifluoroacetate salt (Dios-Arg, 2H-Cho-Arg, or Cho-Arg), or a mixture thereof.

Embodiment 67. The expression vector of any one of Embodiments 64 to 66, wherein the PEG-lipid is DMG-PEG, DSG-PEG, mPEG-DPPE, DOPE-PEG, mPEG-DMPE, mPEG-DOPE, DSPE-PEG-amine, DSPE-PEG, mPEG-DSPE, PEG PE, m-PEG-Pentacosadiynoic acid, bromoacetamido-PEG, amine-PEG, azide-PEG, or a mixture thereof.

Embodiment 68. A composition comprising an expression vector of any one of Embodiments 29 to 67.

Embodiment 69. The composition of Embodiment 68, comprising at least about 0.01, 0.05, 0.1, 0.5, 1, 2, 3, 4, 5, 10, 15, 20, 25, 30, 40, 50, 60, 70, 80, 90, 95% (w/w) the expression vector.

Embodiment 70. The composition of Embodiment 68 or Embodiment 69, further comprising pharmaceutically acceptable excipient, adjuvant, or a combination thereof.

Embodiment 71. The composition of Embodiment 70, wherein the pharmaceutically acceptable excipient comprises a solvent, dispersion media, diluent, dispersion, suspension aid, surface active agent, isotonic agent, thickening or emulsifying agent, preservative, polymer, peptide, protein, cell, hyaluronidase, or mixtures thereof.

Embodiment 72. The composition of Embodiment 70 or Embodiment 71, wherein the adjuvant comprises C34, Gluco-C34, 7DW8-5, C17, C23, C30, α-galactosylceramide (α-GalCer), Aluminum salt (e.g., aluminum hydroxide, aluminum phosphate, alum (potassium aluminum sulfate), mixed aluminum salts), Squalene, MF59, QS-21, Freund's complete adjuvant, Freund's incomplete adjuvant, AS03 (GlaxoSmithKline), MF59 (Seqirus), CpG 1018 (Dynavax), or a mixture thereof.

Embodiment 73. A method for generating an immune response against coronavirus infection, comprising administering an effective amount of a nucleic acid of any one of Embodiments 1 to 28 to a subject in need thereof.

Embodiment 74. The method of Embodiment 73, wherein the nucleic acid is configured as an expression vector of any one of Embodiments 29 to 67.

Embodiment 75. The method of Embodiment 73 or Embodiment 74, wherein the nucleic acid is formulated as a composition of any one of Embodiments 79 to 83.

Embodiment 76. The method of any one of Embodiments 73 to 75, wherein administering the nucleic acid is performed via oral, nasal, mucosal, submucosal, intravenous, intramuscular, intraperitoneal, subcutaneous, intradermal, transdermal, or buccal route.

Embodiment 77. The method of any one of Embodiments 73 to 76, wherein administering is performed 1, 2, 3, 4, 5, 6, 7, 8, 9, or 10 times.

Embodiment 78. The method of Embodiment 77, wherein an interval of each administration to the next administration is about 1, 2, 3, 4, 5, 6, 7 days, about 1, 2, 3, 4, 5, 6, 7, 8, 9, or 10 weeks, or about 1, 2, 3, 4, 5, 6, 7, 8, 9, 10, 11, or 12 months.

Embodiment 79. The method of any one of Embodiments 73 to 78, wherein the coronavirus comprises a SARS-COV, MERS-COV, SARS-COV-2 virus, or a mixture thereof.

Embodiment 80. The method of Embodiment 79, wherein the coronavirus comprises a SARS-COV-2 alpha variant, a SARS-COV-2 beta variant, a SARS-COV-2 delta variant, a SARS-COV-2 omicron variant, or a mixture thereof.

Embodiment 81. The method of any one of Embodiments 73 to 80, wherein the effective amount of the nucleic acid is about 5 μg to 50 μg.

Embodiment 82. The method of any one of Embodiments 73 to 81, wherein the subject is a human.

Embodiment 83. A recombinant protein, comprising an amino acid sequence at least 80%, 85%, 90%, 91%, 92%, 93%, 94%, 95%, 96%, 97%, 98%, or 99%, or 100% identical to SEQ ID NO: 02, wherein X denotes any amino acid, provided that at least one of the X17, X61, X74, X122, X149, X165, X234, X282, X331, X343, X394, X487, X603, X616, and X657 is an Asn (N) residue, and at least one of X709, X717, X801, X1074, X1098, X1108, X1134, X1158, and X1173 is not an Asn (N) residue; SEQ ID NO: 02, wherein X denotes any amino acid, provided that at least one of the X17, X61, X74, X122, X149, X165, X234, X282, X331, X343, X394, X487, X603, X616, X657, X709, X717, and X801 is an Asn (N) N residue, and at least one of the X1074, X1098, X1108, X1134, X1158, and X1173 is not an Asn (N) N residue; SEQ ID NO: 05, wherein X denotes any amino acid, provided that at least one of X23, X31, X115, X388, X412, X422, X448, X472, and X487 is not an Asn (N) residue; SEQ ID NO: 07, wherein X denotes any amino acid, provided that at least one of the X12, X36, X46, X72, X96, and X111 is not an Asn (N) residue; SEQ ID NO: 13, wherein X denotes any amino acid, provided that at least one of the X61, X74, X122, X149, X163, X232, X280, X329, X341, X392, X485, X601, X614, and X655 is an Asn (N) residue, and at least one of the X707, X715, X799, X1072, X1096, X1106, X1132, X1156, and X1171 is not an Asn (N) residue; SEQ ID NO: 13, wherein X denotes any amino acid, provided that at least one of X61, X74, X122, X149, X163, X232, X280, X329, X341, X392, X485, X601, X614, X655, X707, X715, and X799 is an Asn (N) residue, and at least one of X1072, X1096, X1106, X1132, X1156, and X1171 is not an Asn (N) residue; SEQ ID NO: 16, wherein X denotes any amino acid, provided that at least one of X23, X31, X115, X388, X412, X422, X448, X472, and X487 is not an Asn (N) residue; or SEQ ID NO: 18, wherein X denotes any amino acid, provided that at least one of the X12, X36, X46, X72, X96, and X111 is not an Asn (N) residue.

Embodiment 84. The recombinant protein of Embodiment 83, comprising an amino acid sequence at least 80%, 85%, 90%, 91%, 92%, 93%, 94%, 95%, 96%, 97%, 98%, or 99%, or 100% identical to SEQ ID NO: 02, wherein X denotes any amino acid, provided that at least one of the X17, X61, X74, X122, X149, X165, X234, X282, X331, X343, X394, X487, X603, X616, and X657 is an Asn (N) residue, and each one of the X709, X717, X801, X1074, X1098, X1108, X1134, X1158, and X1173 is not an Asn (N) residue; SEQ ID NO: 02, wherein X denotes any amino acid, provided that at least one of the X17, X61, X74, X122, X149, X165, X234, X282, X331, X343, X394, X487, X603, X616, X657, X709, X717, and X801 is an Asn (N) N residue, and each one of the X1074, X1098, X1108, X1134, X1158, and X1173 is not an Asn (N) N residue; SEQ ID NO: 05, wherein X denotes any amino acid, provided that each one of the X23, X31, X115, X388, X412, X422, X448, X472, and X487 is not an Asn (N) residue; SEQ ID NO: 07, wherein X denotes any amino acid, provided that each one of the X12, X36, X46, X72, X96, and X111 is not an Asn (N) residue; SEQ ID NO: 13, wherein X denotes any amino acid, provided that each one of the X61, X74, X122, X149, X163, X232, X280, X329, X341, X392, X485, X601, X614, X655, X707, X715, and X799 is an Asn (N) residue, and at least one of the X1072, X1096, X1106, X1132, X1156, and X1171 is not an Asn (N) residue; SEQ ID NO: 16, wherein X denotes any amino acid, provided that each one of the X23, X31, X115, X388, X412, X422, X448, X472, and X487 is not an Asn (N) residue; or SEQ ID NO: 18, wherein X denotes any amino acid, provided that each one of the X12, X36, X46, X72, X96, and X111 is not an Asn (N) residue.

Embodiment 85. The recombinant protein of Embodiment 83 or Embodiment 84, comprising an amino acid sequence at least 80%, 85%, 90%, 91%, 92%, 93%, 94%, 95%, 96%, 97%, 98%, or 99%, or 100% identical to SEQ ID NO: 03, provided that each one of the Q709, Q717, Q801, Q1074, Q1098, Q1108, Q1134, Q1158, and Q1173 is not an Asn (N) residue.

Embodiment 86. The recombinant protein of Embodiment 83 or Embodiment 84, comprising an amino acid sequence at least 80%, 85%, 90%, 91%, 92%, 93%, 94%, 95%, 96%, 97%, 98%, or 99%, or 100% identical to SEQ ID NO: 04, provided that each one of the Q1074, Q1098, Q1108, Q1134, Q1158, and Q1173 is not an Asn (N) residue.

Embodiment 87. The recombinant protein of Embodiment 83 or Embodiment 84, comprising an amino acid sequence at least 80%, 85%, 90%, 91%, 92%, 93%, 94%, 95%, 96%, 97%, 98%, or 99%, or 100% identical to SEQ ID NO: 14, provided that each one of the Q707, Q715, Q799, Q1072, Q1096, Q1106, Q1132, Q1156, and Q1171 is not an Asn (N) residue.

Embodiment 88. The recombinant protein of Embodiment 83 or Embodiment 84, comprising an amino acid sequence at least 80%, 85%, 90%, 91%, 92%, 93%, 94%, 95%, 96%, 97%, 98%, or 99%, or 100% identical to SEQ ID NO: 15, provided that each one of the Q1072, Q1096, Q1106, Q1132, Q1156, and Q1171 is not an Asn (N) residue.

Embodiment 89. An isolated immunogenic peptide, comprising at least one amino acid sequence selected from a group consisting of: SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, and SEQ ID NO: 39.

Embodiment 90. A recombinant spike protein, comprising a first plurality of peptides, each comprises an amino acid sequence selected from a group consisting of SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33; and a second plurality of peptides, each comprises an amino acid sequence selected from a group consisting of SEQ ID NO: 34, SEQ ID NO: 35, SEQ ID NO: 36, SEQ ID NO: 37, SEQ ID NO: 38, and SEQ ID NO: 39; and wherein each of the first plurality of peptides is shielded by a glycan, and each of the second plurality of peptides is not shielded by a glycan.

Embodiment 91. The recombinant spike protein of Embodiment 90, comprising an amino acid sequence at least 80%, 85%, 90%, 91%, 92%, 93%, 94%, 95%, 96%, 97%, 98%, or 99%, or 100% identical to SEQ ID NO: 08, provided that each one of the Q12, Q36, Q46, Q72, Q96, and Q111 is not Asn (N) residue.

Embodiment 92. The recombinant spike protein of Embodiment 90, comprising an amino acid sequence at least 80%, 85%, 90%, 91%, 92%, 93%, 94%, 95%, 96%, 97%, 98%, or 99%, or 100% identical to SEQ ID NO: 19, provided that each one of the Q12, Q36, Q46, Q72, Q96, and Q111 is not Asn (N) residue.

Embodiment 93. A method for identifying a glycan-shielded conserved peptide of a glycoprotein, comprising: determining and/or establishing a first 3D structure with a glycan profile and a second 3D structure without the glycan profile of the glycoprotein; calculating a relative solvent accessibility (RSA) of an amino acid of the glycoprotein to identify a glycan-shielded amino acid that is exposed in the second 3D structure but shielded in the first 3D structure, based on the first 3D structure and the second 3D structure; comparing amino acid sequences of a plurality of variants of the glycoprotein to identify a conserved sequence; and mapping the result of the RSA calculation and the conserved sequence identified to identify a glycan-shielded conserved peptide, which comprises the conserved sequence with the glycan-shielded amino acid.

Embodiment 94. The method of Embodiment 93, wherein the conserved sequence comprises about 10 to 30 amino acids.

Embodiment 95. The method of Embodiment 94, wherein the conserved sequence comprises about 10 to 20 amino acids.

Embodiment 96. The method of any one of Embodiments 93 to 95, wherein calculating a relative solvent accessibility (RSA) of an amino acid of the glycoprotein identifies a plurality of glycan-shielded amino acids.

Embodiment 97. The method of any one of Embodiments 93 to 96, wherein at least 10%, 20%, 30%, 40%, 50%, 60%, 70%, 80%, 90%, 95% of the amino acids of the glycan-shielded conserved peptide are glycan-shielded amino acids.

Embodiment 98. The method of any one of Embodiments 93 to 97, wherein the RSA is calculated based on a probe radius of 5 to 14 Angstrom.

Embodiment 99. The method of any one of Embodiments 93 to 98, further comprising identifying a glycosylation site of the glycoprotein.

Embodiment 100. The method of any one of Embodiments 93 to 99, wherein the glycoprotein is a spike protein of a coronavirus, an influenza virus, a human immunodeficiency virus, a human papillomavirus, a dengue virus, a Zika virus, an Epstein-Barr virus, a monkeypox virus, an Ebola virus, a Hepatitis B virus, or a Hepatitis C virus.

Embodiment 101. The method of Embodiment 100, wherein the glycoprotein is a spike protein of a SARS-COV, MERS-COV, or SARS-COV-2 virus.

Embodiment 102. The method of Embodiment of Embodiment 101, wherein the plurality of variants of the glycoprotein comprises a SARS-COV-2 alpha variant, a SARS-CoV-2 beta variant, a SARS-COV-2 delta variant, a SARS-COV-2 omicron variant, or a mixture thereof.

Embodiment 103. The method of Embodiment 101 or 102, wherein the glycoprotein is a spike protein of a SARS-COV-2 Wuhan strain or a SARS-COV-2 Delta strain.

Embodiment 104. The method of any one of Embodiments 101 to 103, wherein the spike protein comprises an amino acid sequence at least 80%, 85%, 90%, 91%, 92%, 93%, 94%, 95%, 96%, 97%, 98%, or 99%, or 100% identical to the amino acid sequence set forth in SEQ ID NO: 01 or SEQ ID NO: 12.

What is claimed is:

1. A nucleic acid, configured to encode a recombinant spike protein, wherein the recombinant spike protein has an N-linked glycosylation site in an S1 domain or an S2 domain thereof, provided that a stem region thereof is devoid of an N-linked glycosylation site.

2. The nucleic acid of claim 1, wherein both the S1 domain and the S2 domain of the recombinant spike protein comprise an N-linked glycosylation site.

3. The nucleic acid of claim 2, wherein the recombinant spike protein comprises an N-linked glycosylation site in a receptor binding domain (RBD) thereof.

4. The nucleic acid of claim 1, wherein the stem region comprises an amino acid substitution of asparagine (N) at a N-linked glycosylation sequon (N-Xa-S/T), wherein N denotes an asparagine (N) residue, S denotes a serine(S) residue, T denotes a threonine (T) residue, and Xa in the sequon is any amino acid residue except proline.

5. The nucleic acid of claim 1, wherein the stem region of the recombinant spike protein comprises an amino acid sequence at least 80%, 85%, 90%, 91%, 92%, 93%, 94%, 95%, 96%, 97%, 98%, or 99%, or 100% identical to SEQ ID NO: 07:
LHVTYVPA QEKXFTTAPA ICHDGKAHFP REGVFVSXGT
HWFVTQRXFY EPQIITTDNT FVSGNCDVVI GIVXNTVYDP
LQPELDSFKE ELDKYFKXHT SPDVDLGDIS GIXASVVNIQ
KEIDRLNEVA KNLNESLIDL QELGKYEQYI K,
wherein X denotes any amino acid, provided that each one of the X12, X36, X46, X72, X96, and X111 is not an Asn (N) residue.

6. The nucleic acid of claim 5, wherein the stem region of the recombinant spike protein comprises an amino acid sequence at least 80%, 85%, 90%, 91%, 92%, 93%, 94%, 95%, 96%, 97%, 98%, or 99%, or 100% identical to SEQ ID NO: 08, provided that each one of the Q12, Q36, Q46, Q72, Q96, and Q111 is not Asn (N) residue.

7. The nucleic acid of claim 1, wherein the recombinant spike protein comprises a S2 domain comprising an amino acid sequence at least 80%, 85%, 90%, 91%, 92%, 93%, 94%, 95%, 96%, 97%, 98%, or 99%, or 100% identical to SEQ ID NO: 05, wherein X denotes any amino acid, provided that each one of the X388, X412, X422, X448, X472, and X487 is not an Asn (N) residue, and at least one of the X23, X31, and X115 is an Asn (N) residue.

8. The nucleic acid of claim 1, wherein the recombinant spike protein comprises an amino acid sequence at least 80%, 85%, 90%, 91%, 92%, 93%, 94%, 95%, 96%, 97%, 98%, or 99%, or 100% identical to SEQ ID NO: 02, wherein X denotes any amino acid, provided that at least one of X17, X61, X74, X122, X149, X165, X234, X282, X331, X343, X394, X487, X603, X616, X657, X709, X717, and X801 is an Asn (N) residue, and each one of the X1074, X1098, X1108, X1134, X1158, and X1173 is not an Asn (N) residue.

9. The nucleic acid of claim 1, wherein the stem region of the recombinant spike protein comprises an amino acid sequence at least 80%, 85%, 90%, 91%, 92%, 93%, 94%, 95%, 96%, 97%, 98%, or 99%, or 100% identical to SEQ ID NO: 18, wherein X denotes any amino acid, provided that each one of the X12, X36, X46, X72, X96, and X111 is not an Asn (N) residue.

10. The nucleic acid of claim 9, wherein the stem region of the recombinant spike protein comprises an amino acid sequence at least 80%, 85%, 90%, 91%, 92%, 93%, 94%, 95%, 96%, 97%, 98%, or 99%, or 100% identical to SEQ ID NO: 19, provided that each one of the Q12, Q36, Q46, Q72, Q96, and Q111 is not Asn (N) residue.

11. The nucleic acid of claim 1, wherein the recombinant spike protein comprises a S2 domain comprising an amino acid sequence at least 80%, 85%, 90%, 91%, 92%, 93%, 94%, 95%, 96%, 97%, 98%, or 99%, or 100% identical to SEQ ID NO: 16, wherein X denotes any amino acid, provided that each one of the X23, X31, X115, X388, X412, X422, X448, X472, and X487 is not an Asn (N) residue.

12. The nucleic acid of claim 1, wherein the recombinant spike protein comprises a S2 domain comprising an amino acid sequence at least 80%, 85%, 90%, 91%, 92%, 93%, 94%, 95%, 96%, 97%, 98%, or 99%, or 100% identical to SEQ ID NO: 16, wherein X denotes any amino acid, provided that each one of the X388, X412, X422, X448, X472, and X487 is not an Asn (N) residue, and at least one of the X23, X31, and X115 is an Asn (N) residue.

13. The nucleic acid of claim 1, wherein the recombinant spike protein comprises an amino acid sequence at least 80%, 85%, 90%, 91%, 92%, 93%, 94%, 95%, 96%, 97%, 98%, or 99%, or 100% identical to SEQ ID NO: 13, wherein X denotes any amino acid, provided that at least one of the X61, X74, X122, X149, X163, X232, X280, X329, X341, X392, X485, X601, X614, X655, X707, X715, and X799 is an Asn (N) residue, and each one of the X1072, X1096, X1106, X1132, X1156, and X1171 is not an Asn (N) residue.

14. The nucleic acid of claim 1, wherein the nucleic acid is a messenger RNA (mRNA).

15. An expression vector, comprising the nucleic acid of claim 1.

16. The expression vector of claim 15, wherein the expression vector is a lipid nanoparticle, a liposome, a polymersome, a viral particle, a plasmid, or a bead.

17. The expression vector of claim 16, wherein the expression vector is a lipid nanoparticle, and the lipid nanoparticle comprises a membrane defining an inner space, and wherein the membrane encompasses the nucleic acid, and the membrane is formed with a plurality of lipid components comprising a bi-functional compound, and the bi-functional compound comprises: 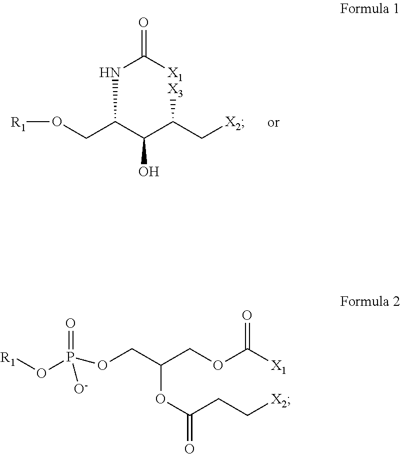
wherein R₁ comprises a substituted or non-substituted glycosyl group;
wherein X₁ and X₂ are each independently hydrogen, C₁₋₃₀ alkyl, C₁₋₃₀ alkenyl, C₁₋₃₀ alkynyl, aryl, aryloxy, or a substituted version thereof, or
—(CH₂)nX₄, n is 0 to 30, and X₄ is hydrogen, aryl, aryloxy, heterocyclic group, or a substituted version thereof, provided that when X₄ is a heterocyclic group, the heterocyclic group comprises 1 to 3 heteroatoms, selected from the group consisting of O, S, and N, or a combination thereof, and
wherein X₃ is hydrogen, C₁₋₆ alkyl, or hydroxyl.

18. The expression vector of claim 17, wherein R¹ is selected from the group consisting of:

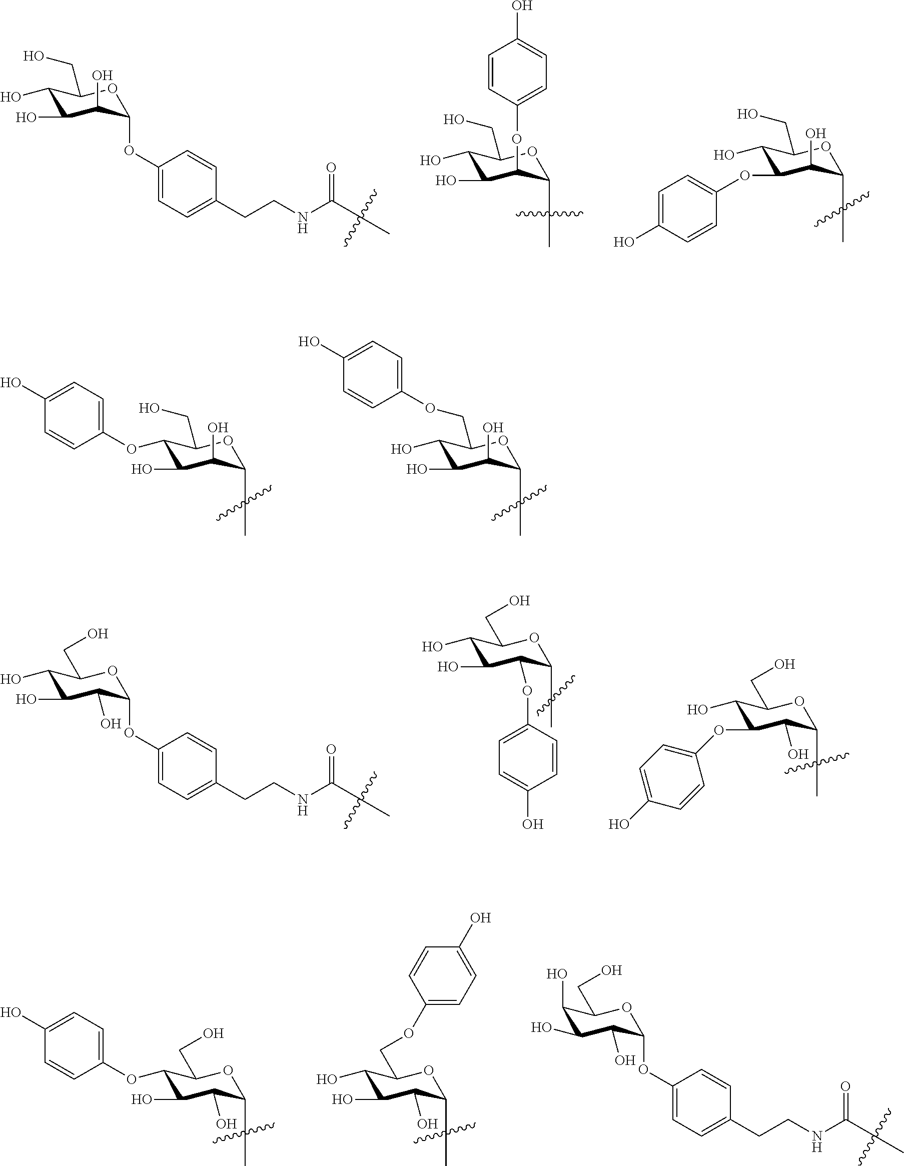

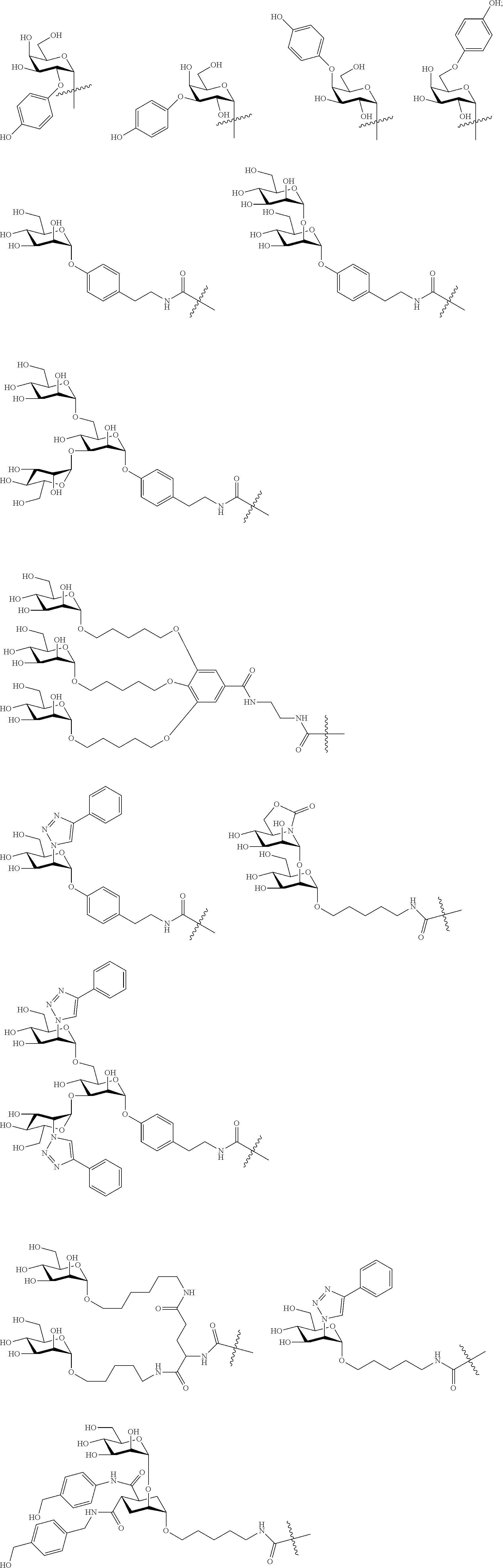

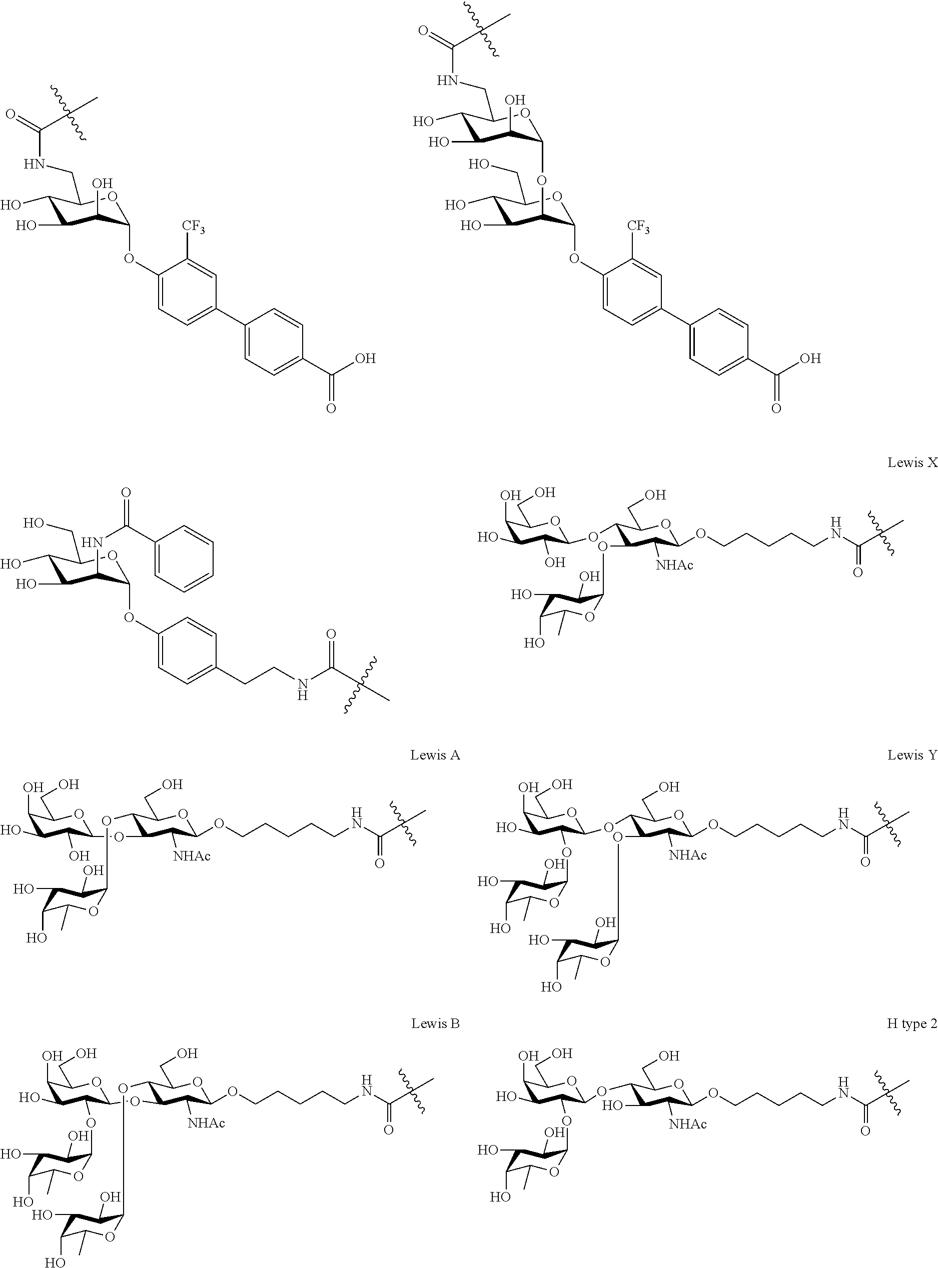

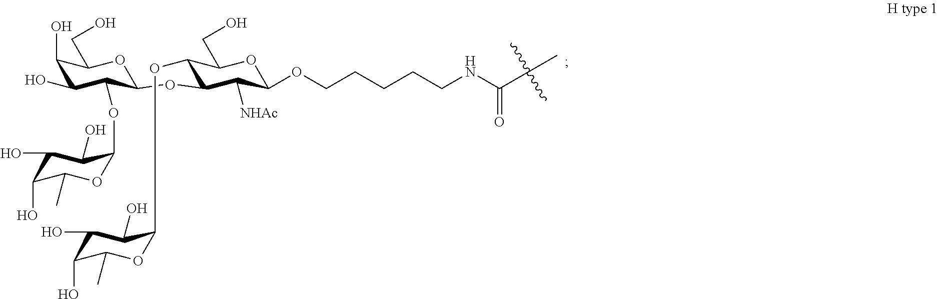

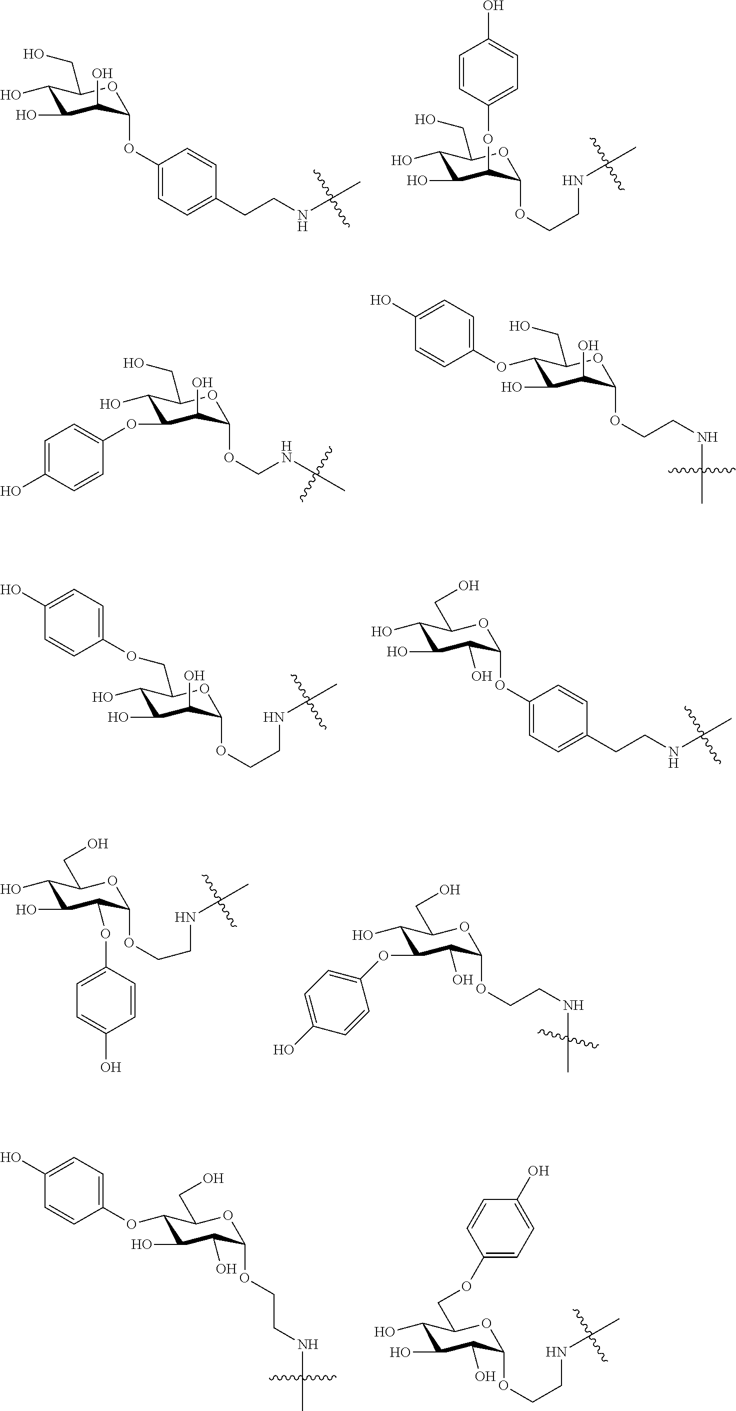

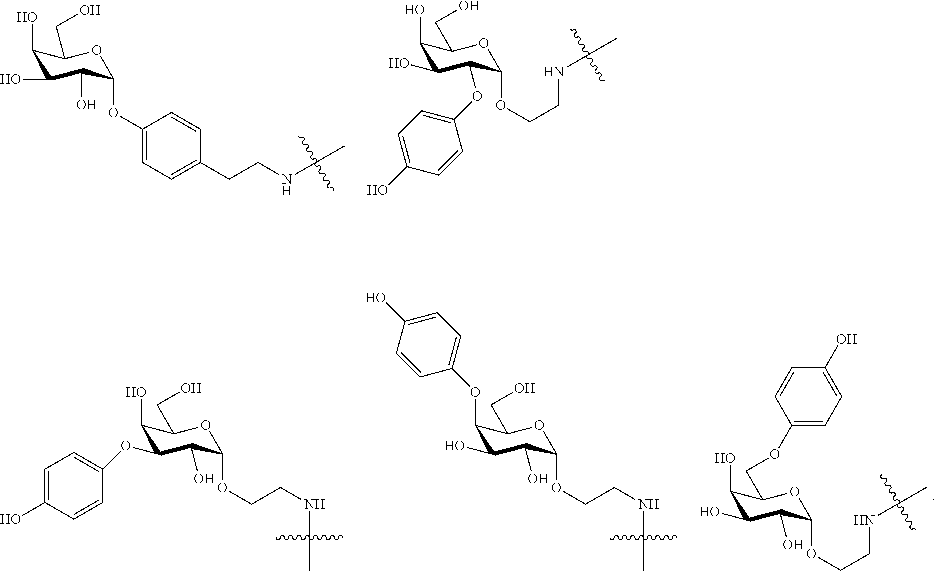

19. The expression vector of claim 17, wherein the compound is selected from the group consisting of:

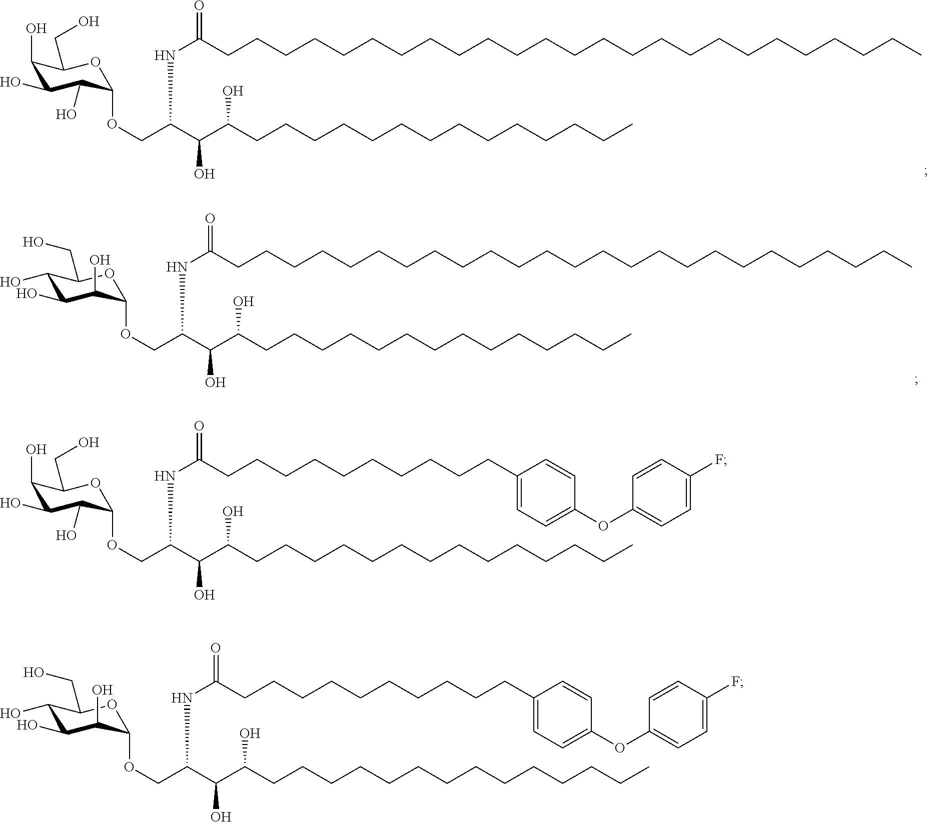

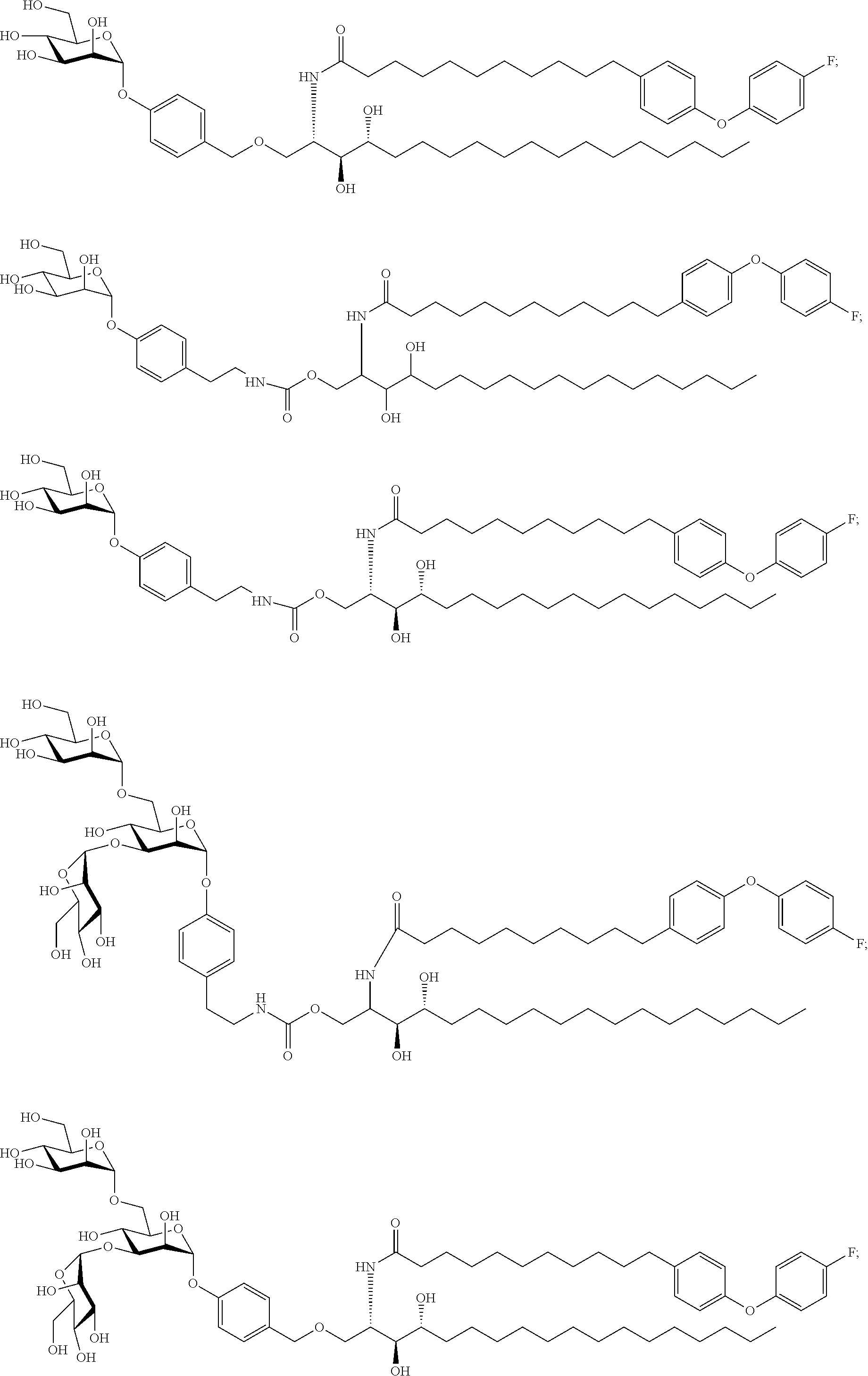

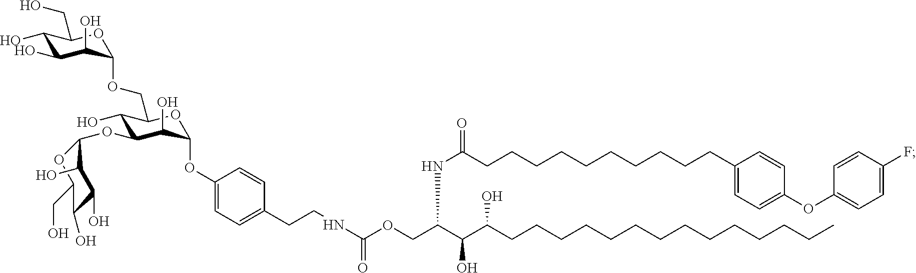

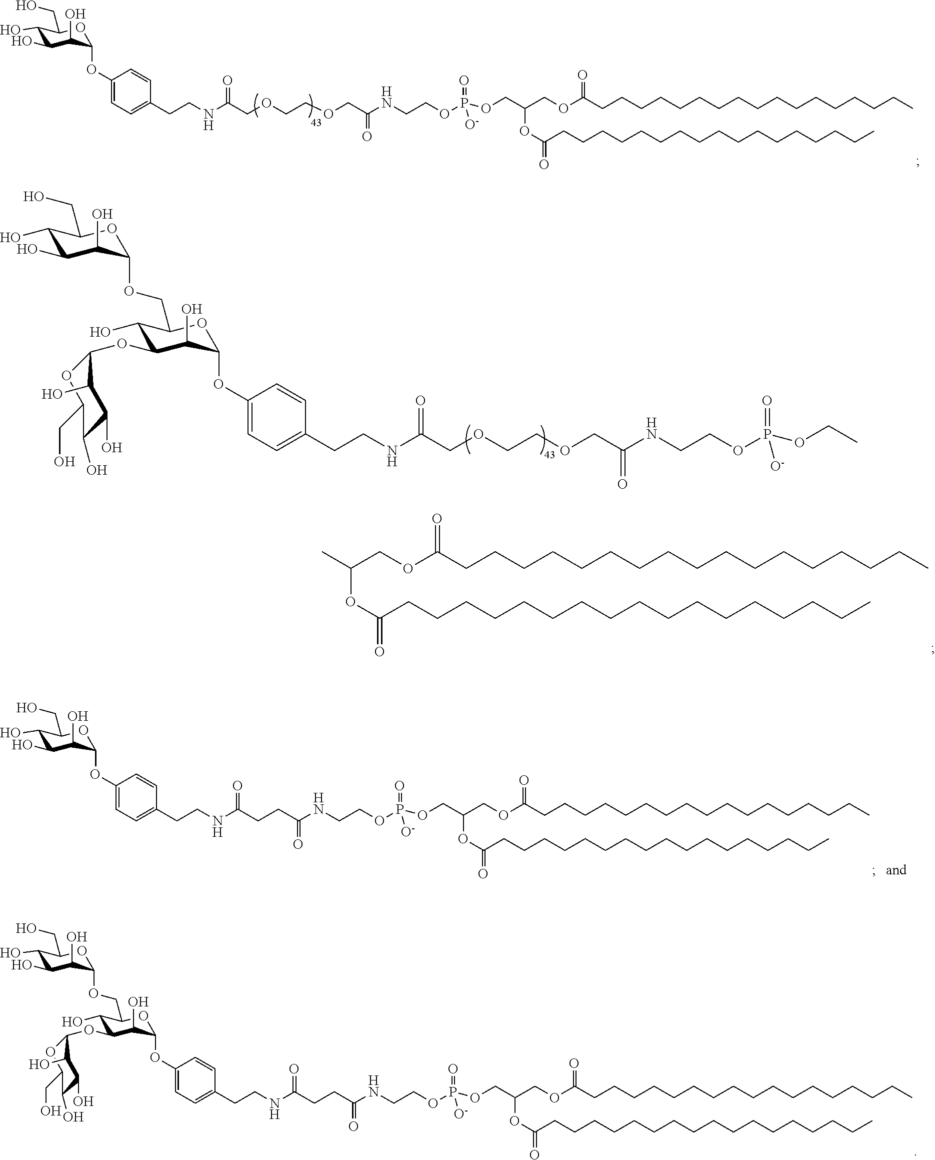

20. A composition comprising an expression vector of claim 15.

21. The composition of claim 20, comprising at least about 0.01, 0.05, 0.1, 0.5, 1, 2, 3, 4, 5, 10, 15, 20, 25, 30, 40, 50, 60, 70, 80, 90, 95% (w/w) the expression vector.

22. The composition of claim 20, further comprising pharmaceutically acceptable excipient, adjuvant, or a combination thereof.

23. A method for generating an immune response against coronavirus infection, comprising administering an effective amount of a nucleic acid of claim 1 to a subject in need thereof.

24. A recombinant protein, comprising an amino acid sequence at least 80%, 85%, 90%, 91%, 92%, 93%, 94%, 95%, 96%, 97%, 98%, or 99%, or 100% identical to
SEQ ID NO: 02, wherein X denotes any amino acid, provided that at least one of the X17, X61, X74, X122, X149, X165, X234, X282, X331, X343, X394, X487, X603, X616, and X657 is an Asn (N) residue, and at least one of X709, X717, X801, X1074, X1098, X1108, X1134, X1158, and X1173 is not an Asn (N) residue;
SEQ ID NO: 02, wherein X denotes any amino acid, provided that at least one of the X17, X61, X74, X122, X149, X165, X234, X282, X331, X343, X394, X487, X603, X616, X657, X709, X717, and X801 is an Asn (N) N residue, and at least one of the X1074, X1098, X1108, X1134, X1158, and X1173 is not an Asn (N) N residue;
SEQ ID NO: 05, wherein X denotes any amino acid, provided that at least one of X23, X31, X115, X388, X412, X422, X448, X472, and X487 is not an Asn (N) residue;
SEQ ID NO: 07, wherein X denotes any amino acid, provided that at least one of the X12, X36, X46, X72, X96, and X111 is not an Asn (N) residue;
SEQ ID NO: 13, wherein X denotes any amino acid, provided that at least one of the X61, X74, X122, X149, X163, X232, X280, X329, X341, X392, X485, X601, X614, and X655 is an Asn (N) residue, and at least one of the X707, X715, X799, X1072, X1096, X1106, X1132, X1156, and X1171 is not an Asn (N) residue;
SEQ ID NO: 13, wherein X denotes any amino acid, provided that at least one of X61, X74, X122, X149, X163, X232, X280, X329, X341, X392, X485, X601, X614, X655, X707, X715, and X799 is an Asn (N) residue, and at least one of X1072, X1096, X1106, X1132, X1156, and X1171 is not an Asn (N) residue;
SEQ ID NO: 16, wherein X denotes any amino acid, provided that at least one of X23, X31, X115, X388, X412, X422, X448, X472, and X487 is not an Asn (N) residue; or
SEQ ID NO: 18, wherein X denotes any amino acid, provided that at least one of the X12, X36, X46, X72, X96, and X111 is not an Asn (N) residue.

25. An isolated immunogenic peptide, comprising at least one amino acid sequence selected from a group consisting of: SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, and SEQ ID NO: 39.

26. A recombinant spike protein, comprising
a first plurality of peptides, each comprises an amino acid sequence selected from a group consisting of SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33; and
a second plurality of peptides, each comprises an amino acid sequence selected from a group consisting of SEQ ID NO: 34, SEQ ID NO: 35, SEQ ID NO: 36, SEQ ID NO: 37, SEQ ID NO: 38, and SEQ ID NO: 39; and
wherein each of the first plurality of peptides is shielded by a glycan, and each of the second plurality of peptides is not shielded by a glycan.


SEQUENCE LISTING (39 sequences)

SEQ ID NO: 1 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1273 aa, synthetic construct
MFVFLVLLPLVSSQCVXLTTRTQLPPAYTNSFTRGVYYPDKVFRSSVLHSTQDLFLPFFSXVTWFHAIHVSGTXGTKRFDNPVLPFNDGVYFASTEKSNIIRGWIFGTTLDSKTQSLLIVNXATNVVIKVCEFQFCNDPFLGVYYHKNXKSWMESEFRVYSSANXCTFEYVSQPFLMDLEGKQGNFKNLREFVFKNIDGYFKIYSKHTPINLVRDLPQGFSALEPLVDLPIGIXITRFQTLLALHRSYLTPGDSSSGWTAGAAAYYVGYLQPRTFLLKYNEXGTITDAVDCALDPLSETKCTLKSFTVEKGIYQTSNFRVQPTESIVRFPXITNLCPFGEVFXATRFASVYAWNRKRISNCVADYSVLYNSASFSTFKCYGVSPTKLNDLCFTXVYADSFVIRGDEVRQIAPGQTGKIADYNYKLPDDFTGCVIAWNSNNLDSKVGGNYNYLYRLFRKSNLKPFERDISTEIYQAGSTPCNGVEGFXCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTXTSNQVAVLYQDVXCTEVPVAIHADQLTPTWRVYSTGSNVFQTRAGCLIGAEHVXNSYECDIPIGAGICASYQTQTNSPRRARSVASQSIIAYTMSLGAENSVAYSXNSIAIPTXFTISVTTEILPVSMTKTSVDCTMYICGDSTECSNLLLQYGSFCTQLNRALTGIAVEQDKNTQEVFAQVKQIYKTPPIKDFGGFXFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKXFTTAPAICHDGKAHFPREGVFVSXGTHWFVTQRXFYEPQIITTDNTFVSGNCDVVIGIVXNTVYDPLQPELDSFKEELDKYFKXHTSPDVDLGDISGIXASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSQNSIAIPTQFTISVTTEILPVSMTKTSVDCTMYICGDSTECSNLLLQYGSFCTQLNRALTGIAVEQDKNTQEVFAQVKQIYKTPPIKDFGGFQFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKQFTTAPAICHDGKAHFPREGVFVSQGTHWFVTQRQFYEPQIITTDNTFVSGNCDVVIGIVQNTVYDPLQPELDSFKEELDKYFKNHTSPDVDLGDISGIQASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKQFTTAPAICHDGKAHFPREGVFVSQGTHWFVTQRQFYEPQIITTDNTFVSGNCDVVIGIVQNTVYDPLQPELDSFKEELDKYFKQHTSPDVDLGDISGIQASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 587 aa, synthetic construct
VASQSIIAYTMSLGAENSVAYSXNSIAIPTXFTISVTTEILPVSMTKTSVDCTMYICGDSTECSNLLLQYGSFCTQLNRALTGIAVEQDKNTQEVFAQVKQIYKTPPIKDFGGFXFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKXFTTAPAICHDGKAHFPREGVFVSXGTHWFVTQRXFYEPQIITTDNTFVSGNCDVVIGIVXNTVYDPLQPELDSFKEELDKYFKXHTSPDVDLGDISGIXASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 587 aa, synthetic construct
VASQSIIAYTMSLGAENSVAYSQNSIAIPTQFTISVTTEILPVSMTKTSVDCTMYICGDSTECSNLLLQYGSFCTQLNRALTGIAVEQDKNTQEVFAQVKQIYKTPPIKDFGGFQFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKQFTTAPAICHDGKAHFPREGVFVSQGTHWFVTQRQFYEPQIITTDNTFVSGNCDVVIGIVQNTVYDPLQPELDSFKEELDKYFKNHTSPDVDLGDISGIQASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 149 aa, synthetic construct
LHVTYVPAQEKXFTTAPAICHDGKAHFPREGVFVSXGTHWFVTQRXFYEPQIITTDNTFVSGNCDVVIGIVXNTVYDPLQPELDSFKEELDKYFKXHTSPDVDLGDISGIXASVVNIQKEIDRLNEVAKNLNESLIDLQELGKYEQYIK

SEQ ID NO: 8 - protein, 149 aa, synthetic construct
LHVTYVPAQEKQFTTAPAICHDGKAHFPREGVFVSQGTHWFVTQRQFYEPQIITTDNTFVSGNCDVVIGIVQNTVYDPLQPELDSFKEELDKYFKQHTSPDVDLGDISGIQASVVNIQKEIDRLNEVAKNLNESLIDLQELGKYEQYIK

SEQ ID NO: 9 - RNA, 3822 nt, synthetic construct
  source 1..3822: mol_type = other RNA
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 10 - RNA, 3822 nt, synthetic construct
  source 1..3822: mol_type = other RNA
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctcagaatagcatcgccatcccaacccagttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttccaattttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacccgccggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagcagttcaccacagcccctgccatctgccacgatggcaaggcccactttccaagggagggcgtgttcgtgtcccagggcacccactggtttgtgacacagcgcaatttctacgagccccagatcatcaccacagacaacaccttcgtgagcggcaactgtgacgtggtcatcggcatcgtgcagaataccgtgtatgatccactccagcccgagctggacagctttaaggaggagctggataagtatttcaagcaacacacctcccctgacgtggatctgggcgacatcagcggcatccaagcctccgtggtgaacatccagaaggagatcgaccgcctgaacgaggtggctaagaatctgcaggagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 11 - RNA, 3825 nt, synthetic construct
  source 1..3825: mol_type = other RNA
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacccgccggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagcagttcaccacagcccctgccatctgccacgatggcaaggcccactttccaagggagggcgtgttcgtgtcccagggcacccactggtttgtgacacagcgcaatttctacgagccccagatcatcaccacagacaacaccttcgtgagcggcaactgtgacgtggtcatcggcatcgtgcaaaataccgtgtatgatccactccagcccgagctggacagctttaaggaggagctggataagtatttcaagcaacacacctcccctgacgtggatctgggcgacatcagcggcatccaggcctccgtggtgaacatccagaaggagatcgaccgcctgaacgaggtggctaagaatctgcaagagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaatga

SEQ ID NO: 12 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTTRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - protein, 1271 aa, synthetic construct
MFVFLVLLPLVSSQCVNLRTRTQLPPAYTNSFTRGVYYPDKVFRSSVLHSTQDLFLPFFSXVTWFHAIHVSGTXGTTRFDNPVLPFNDGVYFASTEKSNIIRGWIFGTTLDSKTQSLLIVNXATNVVIKVCEFQFCNDPFLGVYYHKNXKSWMESGVYSSANXCTFEYVSQPFLMDLEGKQGNFKNLREFVFKNIDGYFKIYSKHTPINLVRDLPQGFSALEPLVDLPIGIXITRFQTLLALHRSYLTPGDSSSGWTAGAAAYYVGYLQPRTFLLKYNEXGTITDAVDCALDPLSETKCTLKSFTVEKGIYQTSNFRVQPTESIVRFPXITNLCPFGEVFXATRFASVYAWNRKRISNCVADYSVLYNSASFSTFKCYGVSPTKLNDLCFTXVYADSFVIRGDEVRQIAPGQTGKIADYNYKLPDDFTGCVIAWNSNNLDSKVGGNYNYRYRLFRKSNLKPFERDISTEIYQAGSKPCNGVEGFXCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTXTSNQVAVLYQGVXCTEVPVAIHADQLTPTWRVYSTGSNVFQTRAGCLIGAEHVXNSYECDIPIGAGICASYQTQTNSRRRARSVASQSIIAYTMSLGAENSVAYSXNSIAIPTXFTISVTTEILPVSMTKTSVDCTMYICGDSTECSNLLLQYGSFCTQLNRALTGIAVEQDKNTQEVFAQVKQIYKTPPIKDFGGFXFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKXFTTAPAICHDGKAHFPREGVFVSXGTHWFVTQRXFYEPQIITTDNTFVSGNCDVVIGIVXNTVYDPLQPELDSFKEELDKYFKXHTSPDVDLGDISGIXASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTTRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSQNSIAIPTQFTISVTTEILPVSMTKTSVDCTMYICGDSTECSNLLLQYGSFCTQLNRALTGIAVEQDKNTQEVFAQVKQIYKTPPIKDFGGFQFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKQFTTAPAICHDGKAHFPREGVFVSQGTHWFVTQRQFYEPQIITTDNTFVSGNCDVVIGIVQNTVYDPLQPELDSFKEELDKYFKQHTSPDVDLGDISGIQASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTTRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKQFTTAPAICHDGKAHFPREGVFVSQGTHWFVTQRQFYEPQIITTDNTFVSGNCDVVIGIVQNTVYDPLQPELDSFKEELDKYFKQHTSPDVDLGDISGIQASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 16 - protein, 587 aa, synthetic construct
VASQSIIAYTMSLGAENSVAYSXNSIAIPTXFTISVTTEILPVSMTKTSVDCTMYICGDSTECSNLLLQYGSFCTQLNRALTGIAVEQDKNTQEVFAQVKQIYKTPPIKDFGGFXFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKXFTTAPAICHDGKAHFPREGVFVSXGTHWFVTQRXFYEPQIITTDNTFVSGNCDVVIGIVXNTVYDPLQPELDSFKEELDKYFKXHTSPDVDLGDISGIXASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - protein, 587 aa, synthetic construct
VASQSIIAYTMSLGAENSVAYSQNSIAIPTQFTISVTTEILPVSMTKTSVDCTMYICGDSTECSNLLLQYGSFCTQLNRALTGIAVEQDKNTQEVFAQVKQIYKTPPIKDFGGFQFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKQFTTAPAICHDGKAHFPREGVFVSQGTHWFVTQRQFYEPQIITTDNTFVSGNCDVVIGIVQNTVYDPLQPELDSFKEELDKYFKQHTSPDVDLGDISGIQASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - protein, 149 aa, synthetic construct; identical to SEQ ID NO: 7

SEQ ID NO: 19 - protein, 149 aa, synthetic construct; identical to SEQ ID NO: 8

SEQ ID NO: 20 - RNA, 3816 nt, synthetic construct
  source 1..3816: mol_type = other RNA
atgttcgtcttcctggtcctgctgcctctggtctcctcacagtgcgtcaatctgcgaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctatcgagaagagcaacatcatcagaggctggatctttggcaccacactggactccaagacacagtctctgctgatcgtgaacaatgccaccaacgtggtcatcaaggtgtgcgagttccagttttgtaatgatcccttcctggacgtgtactatcacaagaacaataagagctggatggagtccggagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatcggtaccggctgtttagaaagagcaatctgaagcccttcgagagggacatctctacagaaatctaccaggccggcagcaagccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcgcagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccagaatgtggtgaatcagaacgcccaggccctgaataccctggtgaagcagctgagcagcaacttcggcgccatctctagcgtgctgaatgacatcctgagccggctggacccgccggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 21 - RNA, 3816 nt, synthetic construct
  source 1..3816: mol_type = other RNA
atgttcgtcttcctggtcctgctgcctctggtctcctcacagtgcgtcaatctgcgaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctatcgagaagagcaacatcatcagaggctggatctttggcaccacactggactccaagacacagtctctgctgatcgtgaacaatgccaccaacgtggtcatcaaggtgtgcgagttccagttttgtaatgatcccttcctggacgtgtactatcacaagaacaataagagctggatggagtccggagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatcggtaccggctgtttagaaagagcaatctgaagcccttcgagagggacatctctacagaaatctaccaggccggcagcaagccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcgcagaagagcccggagcgtggcctcccagtctatcatcgcctataccatgtccctgggcgccgagaacagcgtggcctactctcagaatagcatcgccatcccaacccagttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttccaattttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccagaatgtggtgaatcagaacgcccaggccctgaataccctggtgaagcagctgagcagcaacttcggcgccatctctagcgtgctgaatgacatcctgagccggctggaccgccgggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagcagttcaccacagcccctgccatctgccacgatggcaaggcccactttccaagggagggcgtgttcgtgtcccagggcacccactggtttgtgacacagcgcaatttctacgagccccagatcatcaccacagacaacaccttcgtgagcggcaactgtgacgtggtcatcggcatcgtgcagaataccgtgtatgatccactccagcccgagctggacagctttaaggaggagctggataagtatttcaagcaacacacctcccctgacgtggatctgggcgacatcagcggcatccaagcctccgtggtgaacatccagaaggagatcgaccgcctgaacgaggtggctaagaatctgcaggagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 22 - RNA, 3816 nt, synthetic construct
  source 1..3816: mol_type = other RNA
atgttcgtcttcctggtcctgctgcctctggtctcctcacagtgcgtcaatctgcgcactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctggacgtgtactatcacaagaacaataagagctggatggagtccggcgtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatcgctaccggctgtttagaaagagcaatctgaagcccttcgagagggacatctctacagaaatctaccaggccggcagcaagccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcgcagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccagaatgtggtgaatcagaacgcccaggccctgaataccctggtgaagcagctgagcagcaacttcggcgccatctctagcgtgctgaatgacatcctgagccggctggacccgccggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagcagttcaccacagcccctgccatctgccacgatggcaaggcccactttccaagggagggcgtgttcgtgtcccagggcacccactggtttgtgacacagcgcaatttctacgagccccagatcatcaccacagacaacaccttcgtgagcggcaactgtgacgtggtcatcggcatcgtgcaaaataccgtgtatgatccactccagcccgagctggacagctttaaggaggagctggataagtatttcaagcaacacacctcccctgacgtggatctgggcgacatcagcggcatccaggcctccgtggtgaacatccagaaggagatcgaccgcctgaacgaggtggctaagaatctgcaagagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 23 - protein, 13 aa, synthetic construct
SSANNCTFEYVSQ

SEQ ID NO: 24 - protein, 13 aa, synthetic construct
TESIVRFPNITNL

SEQ ID NO: 25 - protein, 15 aa, synthetic construct
KPFERDISTEIYQAG

SEQ ID NO: 26 - protein, 13 aa, synthetic construct
GPKKSTNLVKNKC

SEQ ID NO: 27 - protein, 11 aa, synthetic construct
TEVPVAIHADQ

SEQ ID NO: 28 - protein, 13 aa, synthetic construct
RVYSTGSNVFQTR

SEQ ID NO: 29 - protein, 10 aa, synthetic construct
RRARSVASQS

SEQ ID NO: 30 - protein, 10 aa, synthetic construct
DPSKPSKRSF

SEQ ID NO: 31 - protein, 12 aa, synthetic construct
FIKQYGDCLGDI

SEQ ID NO: 32 - protein, 12 aa, synthetic construct
ENQKLIANQFNS

SEQ ID NO: 33 - protein, 11 aa, synthetic construct
GKIQDSLSSTA

SEQ ID NO: 34 - protein, 14 aa, synthetic construct
NCDVVIGIVNNTVY

SEQ ID NO: 35 - protein, 19 aa, synthetic construct
PELDSFKEELDKYFKNHTS

SEQ ID NO: 36 - protein, 15 aa, synthetic construct
TSPDVDLGDISGINA

SEQ ID NO: 37 - protein, 14 aa, synthetic construct
VNIQKEIDRLNEVA

SEQ ID NO: 38 - protein, 10 aa, synthetic construct
NLNESLIDLQ

SEQ ID NO: 39 - protein, 11 aa, synthetic construct
LGKYEQYIKWP